(19) United States
(12) Patent Application Publication
(10) Pub. No.: US 2005/0003548 A1
(43) Pub. Date: Jan. 6, 2005

(54) Targeted adenoviral vector displaying immunoglobulin-binding domain and uses thereof

(76) Inventors: Nikolay Korokhov, Birmingham, AL (US); Sam C. Noureddini, Birmingham, AL (US)

Correspondence Address: FROMMER LAWRENCE & HAUG, 745 FIFTH AVENUE- 10TH FL., NEW YORK, NY, 10151, US

(21) Appl. No.: 10/859,739

(22) Filed: Jun. 3, 2004

Related U.S. Application Data
(63) Continuation-in-part of application No. 10/624,317, filed on Jul. 22, 2003.
(60) Provisional application No. 60/398,057, filed on Jul. 22, 2002.

(51) Int. Cl. A61K 48/00; C12N 15/861

(52) U.S. Cl. 435/456; 424/93.2

(57) ABSTRACT

The present invention provides a targeted recombinant adenovirus vector expressing a fiber protein modified by insertion of an immunoglobulin-binding domain that can crosslink to a fusion protein comprising a targeting ligand and an immunoglobulin Zc domain. Interaction between the immunoglobulin-binding domain and the Zc domain results in a targeted vector::ligand complex, thereby targeting the adenovirus vector to a cell that expresses a cell surface molecule that binds to said targeting ligand.

### INCORPORATION BY REFERENCE

This continuation-in-part application claims benefit of U.S. application Ser. No. 10/624,317 filed Jul. 22, 2003, which claims benefit of U.S. provisional application Ser. No. 60/398,057 filed Jul. 22, 2002.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FEDERAL FUNDING LEGEND

This invention was supported in part using federal funds from the U.S. Army Medical Research and Material Command and the National Institutes of Health. Accordingly, the Federal Government has certain rights in this invention.

### FIELD OF THE INVENTION

The present invention relates generally to the targeting of adenoviral vectors. More specifically, the present invention discloses a targeting strategy that involves genetic modifications of the adenoviral capsid and a protein bridge comprising a modified Fc-binding domain of Staphylococcus aureus Protein A.

### BACKGROUND OF THE INVENTION

Adenoviruses (Ad) are a family of over 50 viral pathogens whose non-enveloped protein capsids embody a single copy of double-stranded DNA genome. Based on their ability to agglutinate red blood cells and the homology of their genomes, adenoviruses have been classified into species A through F. The vast majority of the studies of Ad biology have been done on human Ad of serotypes 2 and 5 (Ad2 and Ad5 respectively), both belonging to species C.

The well studied life cycle of adenoviruses, combined with relatively simple methods for the generation, propagation and purification of recombinants derived from Ad2 and Ad5, has made them attractive candidates as gene delivery vectors for human gene therapy. However, two decades of extensive use of Ad-based vectors as prototypes of future gene therapeutics has revealed a number of limitations that have hampered their rapid transition into the clinic. One of these drawbacks is the relative inefficiency of gene delivery by Ad vectors to certain types of diseased human tissues. On the other hand, the susceptibility of many normal tissues to Ad infection makes them random targets for Ad vectors and results in suboptimal distribution of the viruses upon administration to patients.

Attempts to rectify this deficiency of Ad vectors have been rationalized by the identification of the molecular determinants of virus tropism. A typical Ad capsid is an icosahedron, whose planes are formed by the Ad hexon protein while the vertices are occupied by a penton assembly formed by the penton base and protruding fiber proteins. The cell entry mechanism employed by the majority of human Ad serotypes involves two sequential interactions between an Ad particle and a cell. The first of the two contacts involves the Ad fiber protein and the so-called coxsackievirus-adenovirus receptor (CAR). Specifically, the carboxy terminal knob domain of the fiber binds to the immunoglobulin-like D1 domain of CAR, resulting in tight association of the virus with the cell. The presence of CAR on a target cell is thus recognized as a critical prerequisite of efficient infection. This binding step is followed by the secondary contact involving the arginine-glycine-aspartic acid (RGD) sequence found in the Ad penton base protein with cellular integrins avb3 and avb5. This interaction triggers the internalization of the virion within a clathrin-coated endosome. Acidification of the endosome is believed to lead to the release of the virus into the cytoplasm, followed by its translocation to the nucleus where the replication of the virus begins. It has been reported that while CAR is used by the majority of human Ad as a primary receptor, other cell surface molecules are also exploited in this capacity by certain Ad serotypes. This observation suggests that receptor specificity of a given Ad serotype may be modified by redirecting the virus to alternative cellular receptors. This targeting concept has been realized by employing the following strategies. In adapter-mediated targeting, the tropism of the virus is modified by an extraneous targeting moiety, the ligand, which associates with the Ad virion either covalently or non covalently. Adapters or adapter-ligand complexes successfully used for Ad targeting include bispecific antibody (Ab) conjugates, genetic fusions of single chain Ab (scFv) with CAR, or scFv-scFv diabodies (reviewed in Krasnykh & Douglas, 2002, Targeted adenoviral vectors I: Transductional targeting. In Curiel and Douglas ed., Adenoviral Vectors for Gene Therapy. Academic Press, San Diego). Adapter-mediated targeting is rather versatile and technically simple, it may employ a wide range of targeting ligands, and allows for rapid generation of analytical amounts of targeted complexes and their fast validation. However, it requires the production and purification of at least two different components, the virus and targeting ligand, their subsequent conjugation in a targeting complex, and its purification from non-reacted components. These requirements substantially complicate large-scale production of the vector complex, which may result in significant batch-to-batch variations and complicate the regulatory approval of the vector for clinical use.

In contrast, genetic targeting which is based on genetic incorporation of the ligand into the Ad capsid (reviewed in Krasnykh et al., 2000, Mol. Ther. 1:391-405) results in a one-component, self-assembling and self-replicating vector that may be amplified to any desired scale once it is made and validated. The choice of ligands in this strategy, however, is limited to proteins only. Furthermore, additional limitations may be imposed by the potential structural or biosynthetic incompatibility of the ligand with the protein components of Ad capsid. For instance, recent studies showed that certain protein ligands, such as the epidermal growth factor (EGF) or scFvs whose correct folding requires the formation of disulfide bonds, cannot be used for genetic targeting of Ad.

The prior art is deficient in providing a targeting strategy that would overcome the limitations of the above mentioned targeting methods. The present invention fulfills this long-standing need and desire in the art by developing a new approach that combines elements of genetic modification of the Ad capsid with the adaptor-mediated targeting. Ultimately, this new strategy is expected to result in the development of a one-component vector system consists of an Ad vector expressing a secretory form of a targeting ligand that is secreted into the culture medium during Ad vector propagation and is capable of associating with the progeny virions upon cell lysis. This association is possible due to genetic modifications to both the Ad capsid and the ligand, resulting in a mechanism of self-assembly of the vector:ligand targeting complex.

Citation or identification of any document in this application is not an admission that such document is available as prior art to the present invention.

### SUMMARY OF THE INVENTION

A potential barrier to the development of genetically targeted adenovirus (Ad) vectors for cell specific delivery of gene therapeutics lies in the fact that several types of targeting protein ligands require posttranslational modifications, such as the formation of disulfide bonds, which are not available to Ad capsid proteins due to their nuclear localization during assembly of the virion. To overcome this problem the present invention develops a new targeting strategy, which combines genetic modifications of the Ad capsid with a protein bridge approach, resulting in a vector::ligand targeting complex. The components of the complex associate by virtue of genetic modifications to both the Ad capsid and the targeting ligand. One component of this mechanism of association, the Fc-binding domain of Staphylococcus aureus Protein A, is genetically incorporated into the Ad fiber protein. In an advantageous embodiment, a modified Fc-binding domain, the Zc domain, is incorporated into the Ad Fiber protein. The ability of the Zc domain to bind Fab regions of IgG molecules has been abolished with a site directed mutagenesis of a single glycine to alanine substitution. The ligand comprises a targeting component fused with the Fc domain of immunoglobulin that serves as a docking moiety to bind to the genetically modified fibers to form the Ad::ligand complex. The modular design of the ligand, and the fact that it is processed via a secretory pathway, solve the problem of structural and biosynthetic compatibility with the Ad, and thus facilitate targeting the vector to a variety of cellular receptors.

The present study shows that targeting ligands incorporating Fc domain and either an anti-CD40 single chain antibody or CD40L form stable complexes with Protein A modified Ad vectors, resulting in significant augmentation of gene delivery to CD40-positive target cells. As this gene transfer is independent of the expression of native Ad5 receptor by the target cells, this strategy results in the derivation of truly targeted Ad vectors suitable for tissue-specific gene therapy. The novel Fc-binding vector described herein exhibits a significantly high degree of affinity, stability and transduction efficiency when subjected to environments with competing Fc-containing molecules (e.g., the systemic circulation).

The invention encompasses a targeted recombinant adenovirus vector comprising: (i) a gene encoding a heterologous protein, (ii) a modified fiber protein comprising an immunoglobulin-binding domain and (iii) a gene encoding a fusion protein comprising a targeted ligand and an immunoglobulin Zc domain, wherein binding of the immunoglobulin-binding domain to the Zc domain connects the targeting ligand to the modified fiber protein, thereby targeting the adenovirus vector to a cell that expresses a cell surface molecule that binds to the targeting ligand. In another embodiment, the immunoglobulin-binding domain of the targeted adenovirus vector is inserted at the HI loop or the carboxy terminal of the fiber protein. In yet another embodiment, the immunoglobulin-binding domain inserted at the HI loop is flanked by flexible linkers. In another embodiment, the modified fiber protein comprises a fiber-fibritin chimera and the immunoglobulin-binding domain is inserted at the carboxy terminal of the fiber-fibritin chimera. In yet another embodiment, the targeting ligand is a CD40 ligand or a single chain fragment (scFv) of anti-human CD40 antibody.

In another embodiment, the invention provides for a CD40-targeted recombinant adenovirus vector comprising: (i) a gene encoding a heterologous protein, (ii) a modified fiber protein comprising an immunoglobulin-binding domain and (iii) a gene encoding a fusion protein comprising an immunoglobulin Zc domain and a targeting ligand selecting from the group consisting of CD40 ligand and a single chain fragment (scFv) of anti-human CD40 antibody, wherein binding of said immunoglobulin-binding domain to the Zc domain connects the targeting ligand to the modified fiber protein, thereby targeting the adenovirus vector to a CD40+ cell. In one embodiment, the immunoglobulin-binding domain is inserted at the HI loop or the carboxy terminal of the fiber protein. In yet another embodiment, the immunoglobulin-binding domain inserted at the HI loop is flanked by flexible linkers. In another embodiment, the modified fiber protein comprises a fiber-fibritin chimera and the immunoglobulin-binding domain is inserted at the carboxy terminal of the fiber-fibritin chimera. In one embodiment, the CD40+ cell is a dendritic cell. In another embodiment, the gene encoding the heterologous protein and the gene encoding the fusion protein are operably linked to a dendritic-cell-specific promoter.

In a preferred embodiment, the adenovirus vector is the Ad5-Zc1 vector of SEQ ID NO. 15.

The invention also provides for a method of gene transfer to CD40+ cells comprising contacting the CD40+ cells with the above-described targeted adenovirus vectors, wherein the targeted adenovirus vectors mediate transfer of the gene encoding the heterologous protein to the cell. In a preferred embodiment, the CD40+ cells are dendritic cells.

The invention encompasses a method of increasing the binding affinity to a target ligand comprising contacting the target ligand with any one of the above-described targeted adenovirus vectors. The invention also provides for a method of increasing the transduction effiency comprising administering any one of the above-identified targeted adenovirus vectors.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 depicts the analysis of the transiently expressed fiber-Cd (C domain) proteins. 293T/17 cells transfected with pVS-derived expression plasmids were lysed and aliquots of the lysates containing 5 μg of total soluble protein were loaded on an SDS-PAGE gel in sample buffer. The fiber proteins in some of the samples were fully denatured by heating for 5 min at 96° C. (lanes b). These samples were expected to contain the fiber monomers only. In parallel, similarly prepared samples analyzed under “semi-native” conditions were not heat-denatured (lanes a) and were supposed to contain the fiber-Cd proteins in a trimeric configuration. Upon separation, the proteins were electroblotted onto PVDF membrane and probed with anti-fiber tail mAb 4D2.

FIGS. 2A and 2B depict the assessment of the Fc- and CAR-binding ability of the transiently expressed fiber-Cd proteins. The bait proteins, Fc-G28.5 (FIG. 2A) or recombinant CAR (FIG. 2B), adsorbed on ELISA plates were probed with serial dilutions of lysates of fiber-Cd expressing 293T/17 cells. The quantity of the recombinant fibers used in the assay were normalized according to the concentration of total soluble protein in the lysates. The bait-bound fibers were then detected with anti-fiber mAb followed by HRP-conjugated antimouse immunoglobulin G antibodies.

FIGS. 3A and 3B depict the characterization of Ad virions incorporating fiber-Cd proteins. FIG. 3A shows Western blotting of Cd-modified Ad. Aliquots equal to 1010 vp of CsCl-purified Ad vectors were boiled in the sample buffer and their protein components were separated on an SDS-PAGE gel. The fibers electrotransferred onto a membrane were identified with anti-fiber tail mAb 4D2. Lane 1, Ad5.DR-HI-Cd; lane 2, Ad5.DR-HI10-Cd; lane 3, Ad5.DR-HI40-Cd; lane 4, Ad5.DR-HI80-Cd; lane 5, Ad5.DR-LL-Cd; lane 6, Ad5.DR. FIG. 3B shows binding of Cd-containing Ad vectors to Fc-modified targeting ligand. The ligand, Fc-G28.5, was adsorbed on an ELISA plate and incubated with aliquots of the purified Cd-modified Ad virions ranging from 1×10⁹ to 3×10¹¹ vp. Fc-bound Ad particles were detected with anti-Ad2 polyclonal antibodies.

FIGS. 4A and 4B depict the ligand-mediated transduction of CD40 positive cell targets. 293.CD40 (FIG. 4A) or 293 (FIG. 4B) cells preincubated with either Ad5 fiber knob protein, fiber knob and Fc-G28.5 protein, or plain medium were infected with each of the Cd-modified vectors at an MOI of 10 vp/cell. Ad5.DR vector incorporating wild type Ad5 fibers was used as a control. The bars correspond to the luciferase activity in relative light units (RLU) detected in transduced cells 24 hrs post infection (average activity obtained in three replicates). The error bars show standard deviations.

FIGS. 5A and 5B depict the incorporation of Fc-G28.5 fusion protein into targeting vector complexes. Targeting complexes formed by association of the Fc-G28.5 ligand with either Ad5.DR-HI10-Cd, Ad5.DR-HI40-Cd, or Ad5.DR-LL-Cd were purified from unincorporated ligands on CsCl gradients and aliquots of each preparation corresponding to 1.5×10⁹ vp were analyzed by immunoblotting alongside samples of Ad vectors which were not incubated with Fc-G28.5. FIG. 5A shows the membrane probed with anti-fiber mAb, FIG. 5B demonstrates the result of the ligand detection done with Penta-His mAb. “+” indicates the samples pre-incubated with the ligand; “−” shows those containing the Ad vectors only; C, a mixture of 1.5×10⁹ vp of Ad5.DR with 12ng of Fc-G28.5.

FIGS. 6A and 6B depict the transduction of cells by the preformed targeted vector complexes. CD40-negative 293 (FIG. 6A) or CD40-positive Namalwa (FIG. 6B) cells were infected with either Ad5.DR-HI40-Cd, or Ad5.DR-LL-Cd at an MOI of 10 vp/cell or 500 vp/cell respectively. Each of the Cd-modified vectors was used either alone (indicated by “−”) or in association with the Fc-G28-5 ligand (shown by “+”). Ad5.DR was used as an unmodified vector control. The infection was done with or without recombinant Ad fiber knob protein being added to the incubation mixture. Luciferase activity in the transduced cells is shown as either the percentage of the activity detected in unblocked samples (FIG. 6A), or in RLU (FIG. 6B). Standard deviations are represented by the error bars. Of note, the absolute values of luciferase activity in 293 cells infected with targeted vectors were significantly lower than those seen upon infection with untargeted viruses.

FIG. 7 depicts ligand-mediated inhibition of gene transfer by Ad5.DR-LL-Cd::Fc-G28.5 vector complex. CD40-positive Namalwa cells pre-incubated with medium alone or with increasing concentrations of the Fc-G28.5 ligand were transduced with the preformed Ad5.DR-LL-Cd::Fc-G28.5 vector at an MOI of 100 vp/cell. Ad5.DR vector containing unmodified fiber was used as a negative control. Luciferase activity detected in the lysates of cells transduced with the viruses in the presence of competing ligand protein was normalized to that in the cells infected in the absence of free Fc-G28.5. The data points represent the results of three independent determinations with the error bars corresponding to standard deviations.

FIG. 8 depicts targeted transduction of human monocyte-derived dendritic cells. Dendritic cells derived from human monocytes were transduced with either Ad5.DR (shown as Fb wt) or Cd-modified Ad5.DR-LL-Cd vector. In the latter instance, the vector was used in either the untargeted form or pre-complexed with one of the targeting ligands, Fc-G28.5 or Fc-CD40L. Recombinant Ad5 fiber knob or/and Fc-G28.5 proteins were added to some samples to block the interaction between the virus and the CAR or CD40, respectively. Each data point is an average of two measurements. The error bars show standard deviations.

FIGS. 9A and 9B depict overall design of CD40-targeted Ad vector. FIG. 9A shows Ad virion and fiber:ligand complex. Each of the three polypeptides constituting the fiber trimer contains a protein tag (C-domain of S. aureus protein A) incorporated within its knob domain. Similarly, each ligand molecule (TNF-like domain of CD40L or anti-CD40 scFv) contains a complementary tag (Fc-domain). Interaction between the two complementary tags results in cross-linking the virus with the ligand. Only one fiber polypeptide is shown as tag-modified. FIG. 9B shows the genome of PSMA-expressing, CD40-targeted Ad vector. The E1 and E3 regions of the Ad genome are replaced with a double expression cassette containing prostate-specific membrane antigen (PSMA)-and ligand-encoding genes, and the green fluorescent protein (GFP) gene, respectively. The wild type fiber gene is modified to express a tagged form of the fiber protein.

FIGS. 10A-10D depict assessment of Fc and F(ab) binding for recombinant Cd/Zc-modified fiber proteins and Cd/Zc modified Ad5 virions. Transiently expressed Cd- or Zc-modified fibers were used to probe the target ligands, (A) human Fc or (B) human Fab, adsorbed on ELISA plates. Additionally, recombinant Cd- and Zc-modified virions were used to probe either (C) human Fc or (D) human Fab. Bound fiber proteins were detected with anti-fiber antibody, 4D2, while viral vectors were detected with anti-Ad2 polyclonal antibodies. OD490 represents optical density readings measured at 490 nm.

FIGS. 11A-11C depict determination of capsid fiber incorporation with presence of targeting ligand in Ad.Zc and Ad.Cd (previously known as Ad5.DR-LL-Cd) preformed complexes. Western blot of CsCl purified Ad::ligand complexes and wild type virus (Ad.Wt) mixed with targeting ligand, probed with anti-fiber tail mAb 4D2 (A), Penta-His mAb (B) or rabbit anti-mouse polyclonal antibodies (C), to verify presence of fiber, Fc-scFv (Fc-G28.5) fusion protein or anti-CD40 IgG, respectively. Aliquots of 5×10⁹ vp were boiled in Lemmli sample buffer and were separated by electrophoresis on SDS-polyacrylamide gel.

FIG. 12 depicts pH-dependent stability of Ad.Cd virions complexed with various IgG molecules. Human IgG1, human IgG3 and murine IgG3 were adsorbed on ELISA plates and probed with Ad5.Cd vectors in variable pH conditions, to determine stability of viral-IgG complexes. After viral incubation, wells were washed with buffers at pH 5.3 or 7.5. Viral-IgG complexes were then detected with an anti-adenoviral antibody, followed by horseradish peroxidase-conjugated anti-murine antibodies, with subsequent optical density readings measured at 490 nm.

FIG. 13 depicts gene transfer analysis for Ad.Cd pre-formed complexes on 293.CD40 cells. A 24 well, poly-lysine plate containing 293.CD40 cells was pre-incubated on ice with media containing Ad5 knob, and, either Fc-G28.5 fusion protein or anti-CD40 IgG. The cells were then infected with Ad5.GFP (wild-type), Ad.Cd::Fc-G28.5 or Ad.Cd::Fc-IgG at an MOI of 40 vp/cell (2×10⁷ vp). After 48 hours of incubation, GFP expression by live cells was assessed via FACS analysis, and is displayed here as percent-GFP positive cells on analysis of 5×10⁵ cells.

FIG. 14 depicts gene transfer analysis for Ad.Zc pre-formed complexes on 293.CD40 cells. A 24 well, poly-lysine plate containing 293.CD40 cells was pre-incubated on ice with media containing Ad5 knob, and, either Fc-G28.5 fusion protein or anti-CD40 IgG. The cells were then infected with Ad5.GFP (wild-type), Ad.Zc::Fc-G28.5 or Ad.Zc::Fc-IgG at an MOI of 40 vp/cell (2×10⁷ vp). After 48 hours of incubation, GFP expression by live cells was assessed via FACS analysis, and is displayed here as percent-GFP positive cells on analysis of 5×10⁵ cells.

FIG. 15 depicts gene Transfer analysis for Ad.Cd/Zc pre-formed complexes incubated with human-IgG1, on 293.CD40 cells. A 24 well, poly-lysine plate containing 293.CD40 cells was pre-incubated on ice with media containing Ad5 knob. Working dilutions (2×10⁷ vp) of Ad.Cd, Ad.Zc, and pre-complexed, Ad.Cd::Fc-G28.5 and Ad.Zc::Fc-G28.5, were incubated with 30 μg of human-IgG1for 30 minutes at room temperature. The cells were subsequently infected with the viral aliquots, and after 48 hours of incubation, GFP expression by live cells was assessed via FACS analysis, and is displayed here as percent-GFP positive cells on analysis of 5×10⁵ cells.

FIG. 16 depicts gene transfer analysis for Ad.Cd/Zc pre-formed complexes pre-incubated with human-Fc or human-Fab, on 293-CD40 cells. A 24 well, poly-lysine plate containing 293.CD40 cells was prepared for gene transfer analysis. Working dilutions (2×10⁷ vp) of Ad.Cd, Ad.Zc, and pre-complexed, Ad.Cd::Fc-G28.5 and Ad.Zc::Fc-G28.5, were incubated with 30 μg of human-Fc or human-Fab for 30 minutes at room temperature. The cells were subsequently infected with the viral aliquots, and after 48 hours of incubation, GFP expression by live cells was assessed via FACS analysis, and is displayed here as percent-GFP positive cells on analysis of 5×10⁵ cells.

FIG. 17 depicts diagrams of Ad5.Zc, Ad5.PSMA-GFP.w/oFB and Ad5.CEA-GFP.FF-CD40L.

### DETAILED DESCRIPTION OF THE INVENTION

The present invention describes an adenoviral vector targeting approach that combines the advantages of the previously established protein bridge-mediated and genetic modification of virus tropism. It is an object of the present invention to develop an Ad vector system in which genetic modifications done to both the Ad vector capsid and secretory ligand would allow them to self-associate into a stable complex.

This approach was dictated by the major limitation to genetic targeting of Ad, which otherwise remains the most straightforward and efficient way to modify Ad tropism. This limitation is the structural and biosynthetic incompatibility of the protein components of Ad capsid, including the receptor-binding fiber, with certain types of protein molecules that could be attractive candidates as Ad targeting ligands. These candidate proteins include a number of naturally existing molecules (both secretory and anchored within the cell membrane) that require extensive posttranslational modifications that are not available to the Ad proteins localized within the nucleus of infected cells. The major structural feature which limits the use of these proteins as Ad ligands is the presence of the disulfide bonds in their molecules. These disulfide bonds can only be formed in the oxidative environment of the endoplasmic reticulum (ER) by disulfide isomerases, which are residents of the ER. Soon after translation, the fiber and other proteins constituting the Ad capsid traffic to the nucleus whose reducing environment prevents the formation of disulfide bonds. Obviously, the same would hold true for any extraneous protein genetically fused with the fiber. Redirecting the fiber to endoplasmic reticulum, although technically feasible, does not solve the problem as the fiber is then excluded from the assembly of the progeny Ad virions that takes place in the nucleus. These considerations and limitations were proven lately in a report that showed two types of ligands containing disulfide bonds, the epidermal growth factor and scFv, cannot be genetically fused with the functional fiber.

This incompatibility of desired targeting ligands with Ad proteins is resolved in the present work by allowing the virus and the ligand to follow their natural biosynthetic pathways in a non-conflictual manner and, upon proper folding and assembly, associate in a functional vector complex. Data presented herein establish the feasibility of this concept by showing that individual components of such a binary system may be engineered and then put together to form a targeted vector. In one embodiment, the molecular constituents for self-assembly used in the present study are the Fc domain of human immunoglobulin and the Fc-binding domain of Staphylococcus aureus Protein A, which are used to modify the ligand and the virus respectively. In an advantageous embodiment, the modified Fc binding domain, the Zc domain, is incorporated into Ad. The natural affinity of the Protein A for Fc underpins the targeted complex formation. The 59 amino acids long domain C of Protein A was incorporated into either the HI loop or the carboxy terminus of Ad5 fiber to create a docking site for a Fc-modified targeting ligand. None of the modifications affected the yield or the growth dynamics of the resultant Ad vectors. The engineered fibers could be incorporated into mature Ad virions very efficiently. Apparently, none of these modifications caused any significant changes in the folding of the fiber, as its binding to natural Ad receptor, CAR, which requires the involvement of amino acid residues localized on two knob subunits, was not affected. The Fc domain of Ig fused with the ligand served a double duty: in addition to being a facilitator for the expression and secretion of the ligand, it also functioned as an element of the two-component mechanism mediating the association of the ligand with the virus. The Fc domain of Ig has long been used for the purposes of recombinant protein expression. Its incorporation into a protein of interest normally results in a substantial increase in the yield of the protein and also greatly simplifies the purification of the fusion protein on Protein A-containing matrixes. Thus, the use of Fc domain in the present study allowed one to produce secretory form of the targeting ligand in substantial amounts and easily purify it by affinity chromatography. When mixed together, the virus and the ligand undergo self-assembly into a targeting complex that can be purified from unincorporated ligand and then stored as a ready-to-use reagent while retaining its gene delivery properties.

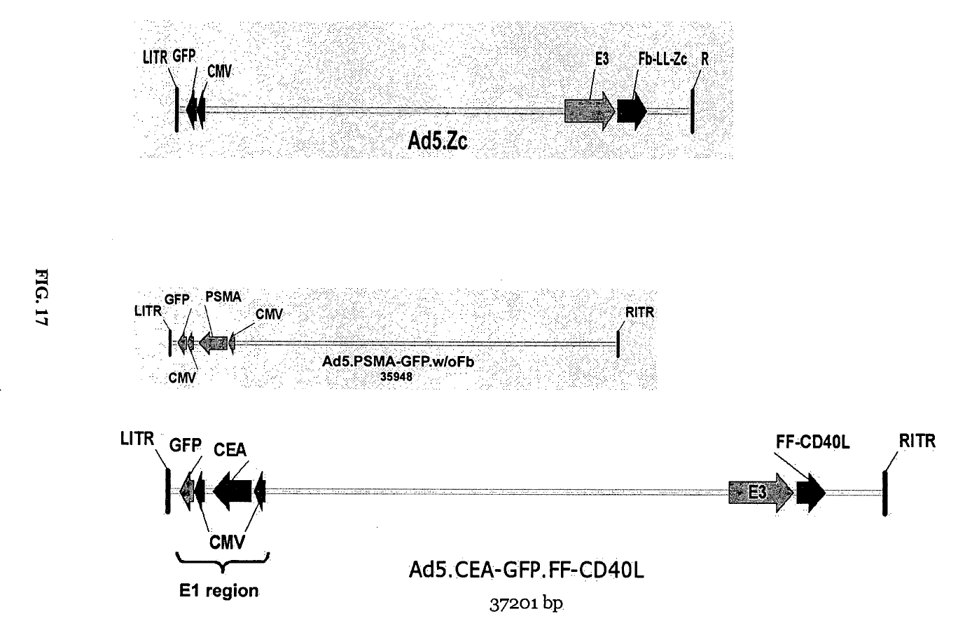

The Zc domain, a mutant form of the C-domain with reduced Fab binding, was generated by replacing the glycine residue at position 29 with alanine by site directed mutagenesis. In a preferred embodiment, the Zc domain was cloned into pKanFb-Cd, resulting in the generation of the shuttle vector pKanFb-Zc. Construction of the pKanFb-Cd and pVS.Fb-Cd plasmids was described previously by Korokhov et al. (see, e.g., Korokhov et al. (2003) J Virol 77: 12931-40). To express the chimeric proteins in mammalian cells, fragments from pKanFb-Cd and pKanFb-Zc were transferred into the expression plasmid pVS.FF/CD40L (see, e.g., Belousova et al. (2002) J Virol 76: 8621-31). Recombinant Ad genomes incorporating the modified fiber genes were derived by homologous DNA recombination in Escherichia coli BJ5183 with SwaI-linearized plasmid pVL4000, as described previously (see, e.g., Chartier et al. (1996) J Virol 70:4805-10). pVL4000 is a derivative of pTG3602 (see, e.g., Chartier et al. (1996) J Virol 70: 4805-10), which contains an Ad5 genome with E1 and the fiber gene deleted. In place of the deleted E1 region, the genome contains a CMV immediate-early promoter driving the green fluorescent protein (GFP) gene. In a preferred embodiment, the vector is Ad5.Zc (FIG. 17). In other embodiments, other E1-deleted vectors can be used, such as Ad5.PSMA-GFP.w/oFB and Ad5.CEA-GFP.FF-CD40L (FIG. 17) can also be used in methods of the invention.

As shown in results from an in vitro gene transfer assay, the pre-formed complexes of Ad with Fc-tagged anti-CD40 scFv or CD40L showed selective gene transfer to target cells via the CD40mediated pathway. Importantly, the present invention demonstrates that association with the targeting ligand results in structural interference with the CAR binding site within the knob, thereby rendering the vector complexes truly targeted. Subsequent use of these CD40-targeted vectors to infect human monocyte-derived dendritic cells demonstrated an augmentation of overall gene transfer that was 30-fold higher than that achieved with an isogenic control Ad incorporating unmodified, wild type fibers, suggesting that the vectors designed in this study may be a more efficient means of delivering antigen-encoding genes to dendritic cells for genetic immunization.

The present invention is a new version of the protein bridge-based targeting approach that offers significant advantages over previously described methods. For instance, by providing a universal solution for the expression of secretory targeting ligands, the targeting approach disclosed herein favorably compares to previously used strategy employing chemical cross-linking of antibodies to form targeting conjugate. Generation of those chemical cross-linked conjugates was proved to be inefficient and thus required large amounts of starting components. Reproducibility in the yields of the cross-linked conjugates is also an issue. The high degree of structural similarity of Ad fiber knob domains from different serotypes predicts the compatibility of Protein A domain C with the frameworks of fiber knobs other than that of Ad5.

The most significant advantage of the strategy described herein is that it allows for the generation of targeted Ad vector in a single infection procedure, wherein the Ad vector modified with the Protein A domain C also expresses the targeting ligand comprising a Fc portion. Targeting complexes self-formed upon cell lysis by the virus progeny will then be isolated by the protocols established for Ad purification. This would significantly simplify the vector manufacturing process and result in high reproducibility and low production costs. The fact that both the virus and the ligand can be produced using the same method, i.e. infection of 293 cells with Ad, strongly supports the feasibility of the proposed approach. While the C domain-modified Ad vectors described herein were designed to be targeted with Fc-ligand fusion proteins, the present invention would be fully suitable for vector targeting utilizing full size antibodies as well.

Applicants hypothesized that Ad.Cd complexed with any Fc-containing targeting ligand, e.g., a whole IgG molecule or the Fc-scFv, would prove to be unstable when placed in environments with competing IgGs, which might displace the targeting ligand for more favorable Cd-Fc interactions, or sterically hinder the targeting ligand from recognizing the desired receptor; such as would be the case in in vivo applications. To a certain extent, this could be accounted for by the ability of Cd to bind the Fab regions common to all IgG molecules, in addition to the Fc domain.

To circumvent this potential problem, Applicants have engineered a novel IgG-binding ligand, the Zc, by modification of the Fc binding domain previously employed for this targeting schema. Based on non-Fab-binding, Fc binding domains (see, e.g., Jansson et al., (1998) FEMS Immunol Med Microbiol 20: 69-78), Applicants have abolished the ability of the C-domain to bind the Fab regions of IgG molecules, and have genetically incorporated the domain at the C-terminus of the Ad5 knob, creating Ad.Zc. Further, Applicants have characterized the ability of these vectors to transfer genes in vitro, via pre-formed complexes with IgG and Fc-scFv. Most importantly, Applicants have shown that only Ad.Zc pre-complexed with Fc-containing ligands, retains its targeting abilities when introduced into environments with competing immunoglobulins. Herein, Applicants' offer a fundamental and critical improvement to Applicants' previous Fc-binding adenoviral technology, optimizing this targeting schema for further application. The novel Fc-binding vector described herein exhibits a significantly higher degree of affinity, stability and transduction efficiency when subjected to environments with competing Fc-containing molecules (e.g., the systemic circulation).

Single chain V region fragments (“scFv”) can also be produced. Single chain V region fragments are made by linking L (light) and/or H (heavy) chain V (variable) regions by using a short linking peptide (Bird et al. (1988) Science 242:423). Any peptide having sufficient flexibility and length can be used as a linker in a scFv. Usually the linker is selected to have little to no immunogenicity. An example of a linking peptide is (GGGGS)₃ (SEQ ID NO. 1) which bridges approximately 3.5 nm between the carboxy terminus of one V region and the amino terminus of another V region. Other linker sequences can also be used, and can provide additional functions, such as for attaching a drug or a solid support.

All or any portion of the H or L chain can be used in any combination. Typically, the entire V regions are included in the scFv. For instance, the L chain V region can be linked to the H chain V region. Alternatively, a portion of the L chain V region can be linked to the H chain V region or a portion thereof. Also contemplated are scFvs in which the H chain V region is from H11, and the L chain V region is from another immunoglobulin. It is also possible to construct a biphasic, scFv in which one component is any target polypeptide and another component is a different polypeptide, such as a T cell epitope.

The scFvs can be assembled in any order, for example, V_{H}-(linker)-V_{L} or V_{L}-(linker)-V_{H}. There may be a difference in the level of expression of these two configurations in particular expression systems, in which case one of these forms may be preferred. Tandem scFvs can also be made, such as (X)-(linker)-(X)-(linker)-(X), in which X are target polypeptides, or combinations of the target polypeptides with other polypeptides. In another embodiment, single chain antibody polypeptides have no linker polypeptide, or just a short, inflexible linker. Exemplary configurations include V_{L}-V_{H} and V_{H}-V_{L}. The linkage is too short to permit interaction between V_{L} and V_{H} within the chain, and the chains form homodimers with a V_{L}/V_{H} antigen-binding site at each end. Such molecules are referred to in the art as “diabodies”.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector is pcDNA3 (Invitrogen, San Diego, Calif.).

Humanized antibodies can also be used for methods of the invention. Humanized forms of non-human (e.g. murine) antibodies are specific chimeric immunoglobulins, immunoglobulin chains, or fragments thereof (such as Fv, Fab, Fab′, F(ab′).sub.2 or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin.

The present invention would be useful in the development of genetic anti-cancer immunization. The development of anti-cancer vaccination strategies has been rationalized by the recent identification of tumor associated antigens (TAA) which may be recognized by the immune system as specific markers of cancer cells, thereby identifying these cells as the targets. These tumor associated antigens include proteins encoded by genes with mutations or rearrangements unique to tumor cells, reactivated embryonic genes, tissue-specific differentiation antigens, and a number of other self proteins. However, despite the identification of these targets, development of effective anti-cancer vaccination strategies has been limited to a large extent by the lack of means for successful vaccination against these weak, self-derived antigens. The generation of a potent anti-tumor associated antigen immune response is thus recognized as a key issue in the development of efficient anti-cancer immunization strategies.

The problem of poor immunogenicity of self-derived tumor-associated antigens can be overcome by efficient antigen presentation by dendritic cells. Current understanding of the mechanisms of immune response development suggests that efficient capture and presentation of tumor associated antigens by antigen presenting cells (APCs) is a pivotal step in eliciting strong anti-cancer immunity. In this regard, dendritic cells (DCs), so-called “professional” APCs, play a major role in the induction of an immune response due to their ability to process and present antigen, express high levels of co-stimulatory molecules, and activate both CD4+ and CD8+ naive T lymphocytes.

Dendritic cells represent a heterogeneous population of bone marrow-derived cells present at low numbers in most peripheral tissues, where they continuously sample the antigenic content of their environment by phagocytosis, macropinocytosis and receptor-mediated endocytosis. A captured antigen is then processed intracellularly, being degraded into short peptides that are loaded onto class I and class II major histocompatibility (MHC) molecules for subsequent display on the cell surface. When dendritic cells encounter local inflammatory mediators, such as tumor necrosis factor α (TNFα) or bacterial lipopolysaccharide, they become activated and undergo a series of physiologic changes leading to their terminal differentiation, a process called “dendritic cell maturation”.

Dendritic cell maturation includes redistribution of MHC molecules from intracellular endocytic compartments to the cell surface, a selective decrease of antigen and pathogen internalization activity and a marked increase in surface expression of co-stimulatory molecules for T cell activation. Maturation also entails profound changes in dendritic cell morphology, reorganization of their cytoskeleton and surface expression of several integrins and chemokine receptors that determine their migration from peripheral tissues to secondary lymphoid organs. Thus, dendritic cells serve as initiators of immune response, capturing antigen at portals of entry and delivering it in a highly immunogenic form for efficient display to T cells.

Stemming from their key functions as central mediators of T cell-based immunity, the uses of dendritic cells have been proposed in a number of clinical immunotherapy strategies. In order to increase the efficiency of delivery of tumor associated antigen-encoding genes to dendritic cells, natural mechanisms of virus-mediated transduction of dendritic cells have been employed. To this end, recombinant adenoviral (Ad) vectors have proved to be more efficient in delivering tumor associated antigen-encoding sequences into dendritic cells than traditional transfection methods.

Several years of studies employing Ad vectors for transduction of dendritic cells, however, have resulted in rather controversial data on the efficiency of this method. A critical analysis of the literature reveals that in those instances where significant levels of Ad-mediated gene transfer to dendritic cells was reported, very high multiplicities of infection (MOIs) had to be used. For instance, Dietz et al. (Blood 91:392, 1998) reported high efficiency adenovirus-mediated gene transfer to human dendritic cells using Ad vector at a MOI of 5,000 virions per cell. Similarly, in order to achieve efficient transduction of bone marrow-derived murine dendritic cells with Ad, Kaplan et al. (J. Immunol. 163:699, 1999) used an MOI of 500 infection units per cell, and Rea et al. transduced human dendritic cells at a MOI of 1,000 plaque forming units per cell (J. Virol. 73:10245, 1999). Whereas the need to use such high doses of the vector does not normally constitute a problem in “proof of concept” studies done in a laboratory, it prevents broad application of Ad-transduced dendritic cells as therapeutic vaccines in the clinic. Importantly, the exposure of immature dendritic cells, whose primary biological function is to capture antigen, to a high concentration of Ad vectors may result in the capture of Ad virions by the dendritic cells and elicitation of an anti-Ad rather than the desired anti-TAA immune response expected from the transduction. While these considerations may not present problems with respect to ex vivo immunization of dendritic cells with Ad vectors, they are particularly important in the context of potential application of Ad-mediated transduction of dendritic cells in vivo, where high doses of Ad vectors administered to patients may cause severe side effects due to toxicity (25-29), thereby compromising the efficiency of the treatment. Thus, any significant improvement on Ad vectors' capacity to transduce dendritic cells that would allow utilization of lower viral doses with higher rates of gene transfer would be highly beneficial for the field of genetic immunization.

Recent studies designed to address the resistance of dendritic cells to Ad infection have revealed the molecular basis of this problem. A majority of human Ad utilizes a cell entry pathway that involves the primary cellular receptor, the coxsackievirus-adenovirus receptor (CAR). Expression of CAR below certain threshold levels may be a common reason for the Ad-refractoriness of a variety of cell targets. Specifically, poor efficiencies of gene transfer to dendritic cells by Ad vectors have been shown to correlate with low levels of CAR expression in these cells. Therefore, the dependence of Ad-mediated transduction on the levels of CAR expressed on target dendritic cells represents a major obstacle in using Ad vectors for genetic immunization.

CAR-deficiency of dendritic cells and their refractoriness to Ad infection may be overcome by modification of Ad tropism to target the vector to specific receptors expressed by dendritic cells. Recent studies performed at, the Gene Therapy Center at University of Alabama at Birmingham have clearly demonstrated the efficacy of this tropism modification strategy by targeting the vector to the CD40 receptor present on the surface of dendritic cells. Specifically, by employing a bispecific antibody with affinities for both the adenovirus fiber knob and CD40, a luciferase-expressing Ad vector was re-routed via CD40 that served the role of an alternative primary receptor for Ad binding. The selection of CD40 as an alternative receptor for the Ad vector was rationalized by the fact that this molecule, which play an important role in antigen-presentation by dendritic cells, is efficiently expressed by immature dendritic cells. The CD40-targeted Ad vector increased reporter gene expression in dendritic cells by at least two orders of magnitude as compared to untargeted Ad. Furthermore, this enhancement was blocked by 90% when cells were pretreated with an excess of the unconjugated anti-CD40 monoclonal antibody.

Importantly, this antibody-based targeting resulted in modulation of the immunological status of dendritic cells by inducing their maturation. This was demonstrated phenotypically by increased expression of CD83, MHC, and costimulatory molecules, as well as functionally by production of IL-12 and an enhanced allostimulatory capacity in a mixed lymphocyte reaction (MLR). It has been reported that activation of dendritic cells to maturity renders them resistant to the effects of dendritic cell inhibitory cytokines like IL- 10 as well as to direct tumor-induced apoptosis. The capacity with which murine dendritic cells can generate an immune response in vivo has been shown to correlate with the degree of their maturation. Moreover, based on proposals that CD40 activation may bypass CD4+ T cell help, a CD40-targeted Ad might also have applications in cases of CD4+ dysfunction. The dual role of CD40 in this schema as both a surrogate Ad receptor and a powerful trigger of DC maturation rationalize further development of dendritic cell-targeting Ad vectors for anti-cancer immunization.

Alternatively, an Ad vector may be targeted to CD40 by cross-linking with the natural ligand for CD40 receptor, CD40 Ligand or CD40L. CD40-CD40L interaction is characterized by high affinity and specificity and also launches a cascade of events leading to the initiation of an immune response. The multivalent interaction of trimeric CD40L with CD40 receptors causes CD40 ligation, which then results in enhanced survival of these cells and secretion of cytokines such as IL-1, IL-6, IL-8, IL-10, IL-12, TNF-α , MIP-1a and enzymes such as matrix metalloproteinase. CD40-CD40L interaction also enhances monocyte tumoricidal activity. In addition, ligation of CD40 to CD40L considerably alters dendritic cell phenotype by upregulating the expression of costimulatory molecules such as CD54/ICAM-1, CD58/LFA-3, CD80/B7-1, and CD86/B7-2. Therefore, the interaction between CD40 and CD40L has important consequences for both antigen presenting cell function and T′cell function.

The present invention discloses an Ad vector suitable for selective and efficient gene transfer to dendritic cells or any cell type for which an Fc-containing targeting moiety can be developed, due to the modular nature of Ad.Zc. The targeting system involves interaction between the Fc domain of an antibody and an immunoglobulin-binding domain to cross-link an adenoviral vector to a targeting ligand. The Ad vector is targeted to CD40, which functions as a surrogate viral receptor, by complexing the Ad vector with a CD40-specific protein moiety such as the natural ligand for CD40, CD40L, or an anti-CD40 single chain antibody. A single-chain (scFv) version of anti-human CD40 mAb G28.5 has been derived at the Gene Therapy Center at University of Alabama and its ability to bind CD40 expressed on cell surface has been demonstrated. As this scFv represents the CD40-binding domains of the parental mAb, by all accounts it should retain the capacity of G28.5 to activate dendritic cells upon binding to CD40 and may thus 15 be used as an adequate substitute for the full size mAb in a targeting strategy. Fc domain of an antibody and the C domain of S. aureus protein A (CdpA) are incorporated into the targeting ligand and the Ad fiber protein respectively, and interaction between these two complementary tags results in cross-linking the virus with the targeting ligand. To date, the carboxy terminus and the HI loop within the Ad fiber knob domain have been identified as favoring incorporation of heterologous peptide sequences. Recent work has demonstrated that each of these sites within the fiber can accommodate polypeptide sequences exceeding 70 amino acid residues in length.

In addition to the C domain of S. aureus protein A, one of skill in the art can use other immunoglobulin-binding domains well known in the art.

In addition to attaching an immunoglobulin-binding domain to the fiber protein, the immunoglobulin-binding domain can also be inserted into fiber-fibritin chimera as an alternative strategy. The fiber-fibritin protein was designed so that the structure of the domain providing for trimerization of the chimera (fibritin) is not affected by incorporation of heterologous peptides/polypeptides within the protein, thereby dramatically increasing the odds of obtaining stable derivatives of this “backbone” molecule.

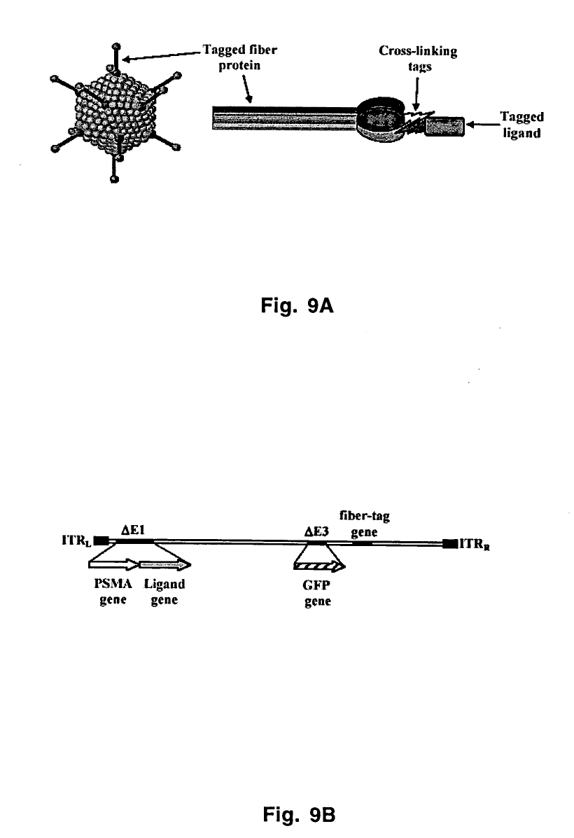

One object of the present invention is to provide targeted adenoviral vectors for uses in immunotherapy. Accordingly, in one embodiment of the present invention, there is provided a highly efficient Ad vectors suited for genetic immunization of humans against prostate cancer (PCA) (FIG. 9). The rationale of this approach is based on the fact that a potent anti-prostate cancer immune response can be induced by selective and efficient delivery to, and expression in, human dendritic cells of a prostate cancer-specific antigen, prostate-specific membrane antigen (PSMA). It is expected that efficient expression of PSMA within dendritic cells, which are highly specialized, professional antigen-presenting cells, would lead to induction of anti-PSMA immune response directed against prostate cancer tumor and eradication of tumor cells by the patient's immune system.

This expectation is based on the following findings. Prostate tumors express tumor-specific antigens (TAAs) that are suitable for development of immune-based therapies (Tjoa & Murphy, 2000, Semin. Surg. Oncol. 18:80-7). Cytotoxic lymphocytes (CTLs) have been generated in vitro against prostate-specific antigen (PSA). Importantly, more recent data demonstrate that PSA-specific cellular immunity can be generated in humans (Meidenbauer et al., 2000, Prostate 43:88-100 (2000)). Immunotherapy has been successfully employed to treat prostate tumors in mouse models. Dendritic cells have been shown to be effective in generating prostate tumor-specific immunity in humans in other contexts as well (Salgaller et al., 1998, Crit. Rev. Immunol. 18:109-19). A recent report suggested that dendritic cells pulsed with mRNA from prostate carcinomas induced significant human immunity that correlated with reduced metastatic tumor transit in blood (Heiser et al., 2002, J Clin. Invest. 109:409-17).

PSMA is a prostate cancer tumor-specific antigen, which is produced by both the prostate cancer tumor cells and the endothelial cells of the prostate cancer tumor vasculature, that is the subject of immune attack by CTLs (Lodge et al., 1999, J Immunother. 22:346-55). Dendritic cells pulsed with PSMA-specific peptides have generated significant short-term clinical responses in human patients, prompting further employment of this tumor-specific antigen in development of immunotherapies for prostate cancer patients (Tasch et al., 2001, Crit. Rev. Immunol. 21:249-61). Interestingly, antibodies directed against PSMA are also effective in treating prostate cancers, with anti-PSMA immunity being associated with tumor clearance in mice. Both cellular and humoral immunity may be important, and dendritic cells are capable of inducing both types of responses. Expression of PSMA by both the prostate tumor cells and prostate vasculature endothelium suggests that genetically induced anti-PSMA immunity will cause the destruction of the tumor directly and also via abrogation of its blood supply, thereby resulting in a synergistic enhancement of the therapeutic effect. Thus, based on these data, strategies to target PSMA expression to dendritic cells may improve the effectiveness of immune-based therapies for cancer of prostate.

The major improvement of the Ad vector disclosed herein compared to the Ad5-based vectors presently used for anti-prostate cancer vaccination is its engineered ability to deliver PSMA to human dendritic cells in a targeted, highly efficient manner. Based on early findings by Tillman et al. (Tillman et al., 1999, J Immunol. 162:6378-83 and Tillman et al., 2000, Cancer Res. 60:5456-63), not only it is expected to dramatically increase the efficiency of dendritic cells transduction by the CD40-targeted Ad, it is also expected that binding of this Ad to CD40 on dendritic cells will trigger their maturation and the ability to activate cytotoxic T cells, thereby 15 leading to development of a potent anti-prostate cancer immune response. The vector of the present invention is engineered to express PSMA, and a secretory, tagged form of a targeting ligand. In its final configuration it will consist of a recombinant form of either CD40L or an anti-CD40 scFv linked via Fc:protein A interaction to an Ad virion encoding PSMA. Of note, the Fc domain-containing ligands will be encoded by the genomes of the same Ad vectors they are designed to associate with and thus retarget. Importantly, in the described configuration this vector will constitute a one-piece, self-assembling delivery vehicle, production of which does not require any additional steps over and above its amplification in a corresponding cell line with subsequent purification. This feature of the proposed system should greatly facilitate large-scale manufacturing of the targeted vector by eliminating the need for production of the vector and the targeting ligand in two separate technological processes.

In view of the present disclosure, one of ordinary skill in the art would readily apply the method of the instant invention to direct adenoviral vectors carrying various heterologous proteins or tumor-specific antigens to targets besides CD40+ cells. Other targeting ligands and heterologous proteins or TAA that are within the scope of the instant invention would be readily recognized by a person having ordinary skill in this art.

In accordance with the present invention there may be employed conventional molecular biology, microbiology, and recombinant DNA techniques within the skill of the art. Such techniques are explained fully in the literature. See e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Flames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The term antibody used herein is intended to encompass both polyclonal and monoclonal antibodies. The term antibody is also intended to encompass whole antibodies, biologically functional fragments thereof, chimeric and humanized antibodies comprising portions from more than one species.

Biologically functional antibody fragments include Fab, Fv, F(ab′)₂ and scFv (single-chain antigen-binding protein) fragments. As used herein, single chain antibodies or scFvs are polypeptides which consist of the variable (V) region of an antibody heavy chain linked to the V region of an antibody light chain with or without an interconnecting linker. This comprises the entire antigen binding site, and is the minimal antigen binding site.

Chimeric antibodies can comprise proteins derived from two different species. The portions derived from two different species can be joined together chemically by conventional techniques or can be prepared as a single contiguous protein using genetic engineering techniques (See, e.g., Cabilly et al., U.S. Pat. No. 4,816,567, Neuberger et al., WO 86/01533 and Winter, EP 0,239,400). Such engineered antibodies can be, for instance, complementarity determining regions (CDR)-grafted antibodies (Tempest et al., Biotechnology 9:266-271 (1991)) or “hyperchimeric” CDR-grafted antibodies which employ a human-mouse framework sequence chosen by computer modeling (Queen et al., Proc. Natl. Acad. Sci. U.S.A. 86:10029-10033 (1989)).

The present invention is directed to a targeted recombinant adenovirus vector comprising (i) a gene encoding a heterologous protein; (ii) a modified fiber protein with an immunoglobulin-binding domain; and (iii) a gene encoding a fusion protein comprising an immunoglobulin Zc domain and a targeting ligand. Binding of the immunoglobulin-binding domain to the Zc domain would connect the targeting ligand to the modified fiber protein, thereby targeting the, adenovirus vector to a cell that expresses a cell surface molecule that binds to the targeting ligand. The modified fiber protein can be a fiber-fibritin chimera. The immunoglobulin-binding domain (for example, the Zc-binding domain of Staphylococcus aureus Protein A) can be inserted at the HI loop or the carboxy terminal of the modified fiber protein. In one embodiment of the present invention, the adenovirus vector is targeted to CD40+ cells, such as dendritic cells, by employing CD40 ligand or a single chain fragment (scFv) of anti-human CD40 antibody as targeting ligand.

The present invention is also directed to a method of gene transfer to CD40+ cells using the CD40-targeted adenoviral vector disclosed herein. In general, the CD40+ cells are dendritic cells.

The following examples are given for the purpose of illustrating various embodiments of the invention and are not meant to limit the present invention in any fashion.

### EXAMPLES

### Example 1

### Cell Lines And Reagents

293 human embryonal kidney cells, their derivative 293T/17 which expresses the simian virus 40 large T antigen, and Namalwa Burkitt's lymphoma human cells were purchased from the American Type Culture. Collection (Manassas, Va.). Namalwa cells were cultured in RPMI medium adjusted to contain 1.5 g/L sodium bicarbonate, supplemented with 2 mM L-glutamine, 4.5 g/L glucose, 1.0 mM sodium pyruvate, and 7.5% fetal bovine serum (FBS). 293 and 293T/17 cells were propagated in Dulbecco's modified Eagle's medium (DMEM)/F-12 medium with 10% FBS, 2 mM glutamine, 100 U/ml penicillin, and 100 mg/ml streptomycin. FBS was purchased from HyClone (Logan, Utah), and media and supplements were from Mediatech (Herndon, Va.). All cells were propagated at 37° C. in a 5% COz atmosphere.

Dendritic cells (DCs) were derived from the peripheral blood of normal donors. Peripheral blood mononuclear cells were purified with gradient centrifugation using Histopaque (Sigma Diagnostics, St. Louis, Mo.). CD 14-positive monocytes were then isolated using CD14 microbeads and magnetic cell sorting (Miltenyi Biotec, Auburn, Calif.). They were cultured for six days in RPMI 1640 medium with 10% FBS, 2 mM glutamine, 100 U/ml penicillin, 100 ug/ml streptomycin, and 50 mM 2-ME containing 100 ng/ml recombinant human IL-4 (R&D Systems, Minneapolis, Minn.) and 100 ng/ml recombinant human GM-CSF (Immunex, Seattle, Wash.). Expression of molecular markers typical of immature DC (CD14 negative; CD11c, CD40, CD86, and HLADR positive) was confirmed by staining with relevant monoclonal antibodies (mAb).

Rabbit anti-Ad2 polyclonal antibodies were purchased from the National Institute of Allergy and Infection Diseases (Bethesda, MD). Anti-mouse and anti-rabbit immunoglobulin polyclonal antibodies conjugated with horseradish peroxidase were from Amersham Pharmacia Biotech Inc. (Piscataway, N.J.) and DAKO (Carpinteria, Calif.), respectively. 4D2 anti-fiber mouse mAb (Hong & Engler, 1996, J Virol. 70:7071-8) was provided by Jeffrey Engler (University of Alabama at Birmingham, Ala.). Penta-His mAb, which binds five histidine sequence was purchased from Qiagen (Valencia, Calif.).

Restriction endonucleases and T4 DNA ligase were purchased from New England Biolabs (Beverly, Mass.). The polymerase chain reaction (PCR) was performed with Pfu DNA polymerase (Stratagene, La Jolla, Calif.)

### Example 2

### Design of AdS Fiber Protein Modified With The C Domain of Staphylococcus aureus Protein A

To design a versatile mechanism for attachment of targeting ligands to Ad particles, the structure of each of these components were modified with distinct protein moieties capable of forming stable heteroduplexes upon association with each other. To this end, the C domain (Cd) of Staphylococcus aureus Protein A was introduced within the fiber protein of the Ad5 vector. This domain is known to bind with high selectivity and affinity to the Fc domain of immunoglobulins (Ig). Therefore, Ad virions incorporating such Cd-modified fibers were expected to bind targeting ligands designed to contain an Fc domain.

A total of five genes coding for different C domain (Cd)-containing fibers were designed by incorporation of the C domain open reading frame into either the carboxy terminus of the fiber protein (Fb-LL-Cd), or into the HI loop of its knob domain. In the latter instance, in addition to direct fusion of the C domain sequence with that of the HI loop (Fb-HI-Cd), three other constructs (Fb-HI10-Cd, Fb-H140-Cd and Fb-H180-Cd) were made in which the C domain was flanked within the loop with flexible linkers derived from the AdS penton base protein (Belousova et al., 2002, J Virol. 76:8621-31). These additional constructs were designed to avoid potential steric hindrance that could be caused by the proximity of the knob to C domain within the fusion molecule. The C domain was extended away from the knob by linkers having 5, 20 or 40 amino acid residues.

### Example 3

### Vectors for AdS Fiber Protein Modified With The C Domain of Staphylococcus aureus Protein A

To facilitate modifications of the HI-loop of AdS fiber, shuttle vector pKanHI-Bael carrying the AdS fiber gene with flanking regions of Ad genomic DNA and the recognition sequence for the restriction endonuclease Bae I within the HI-loop was constructed by a two-step cloning strategy. First, the shuttle vector pKanₚHI was generated by subcloning of the 3.1-kb PmeI-EcoRI fragment of pXKₚHI (Belousova et al., 2002), whose ends were filled-in with the Klenow fragment of DNA polymerase I of E. coli, into ApoI-AflIII-digested pZErO-2 (Invitrogen, Carlsbad, Calif.). Next, a BaeI recognition site within the HI-loop-encoding sequence was generated by cloning the duplex made with oligonucleotides Bae.F (ACAACTCGGTGGCGGTACCGGTGTATACGGCGGTCC, SEQ ID NO. 2) and Bae.R (GGACCGCCGTATACACCGGTACCGCCACCGAGTTGT, SEQ ID NO. 3) into EcoRV-digested plasmid pKanₚHI, resulting in shuttle vector pKanHI-BaeI.

A shuttle vector suitable for modifications of the carboxy terminus of the fiber protein was designed by subcloning an AgeI-MfeI-fragment of the previously described pBS.F5LLBamHI (Krasnykh et al., 1996) into the AgeI-MfeI-digested pKanₚHI. This resulted in plasmid pKanLL-BamHI encoding a modified fiber with a C-terminal peptide linker (G4S)3 followed by a BamHI restriction site. This site was then replaced with the BaeI recognition sequence by inserting a duplex made of two oligonucleotides, LL-Bae-1F (GATCCCGGTGGCGGTACCGGTGTATACGGCGGTTAATAAA,SEQ ID NO. 4) and LL-Bae-1R (GATCTTTATTAACCGCCGTATACACCGGTACCGCCACCGG, SEQ ID NO. 5), thereby generating pKanLL-BaeI.

Plasmid pDV67, which was constructed for the expression of Ad5 fiber and its derivatives in mammalian cells, was described in Von Seggem et al. (Von Seggem et al., 2000, J Virol. 74:354-62). To simplify the transfer of fiber genes assembled within pDV67 into the pKan3.1-derived fiber shuttle vectors, the MfeI restriction site located upstream from the CMV promoter was deleted to make pVSI. A new MfeI site was introduced downstream from the 3′ end of the fiber open reading frame (ORF) by cloning an MfeI-Xbal-linker (CTAGCCAATTGG, SEQ ID NO. 6) into XbaI-digested pVSI, resulting in pVSII.

Recombinant genes encoding the Ad5 fiber modified by incorporation of the C-domain of Staphylococcus aureus Protein A (SpA) within the HI loop and at the carboxy(C)-terminus were assembled in two steps. First, AgeI-MfeI-fragments isolated from the plasmids pKanHIBael, pKan-LL-BaeI, pHI.B1O, pHI.PB40, or pHI.PB80 were cloned into AgeI-MfeIdigested pVSII. Next, the nucleotide sequence encoding the C-domain of SpA was assembled with two pairs of oligonucleotides TI (GCGGATAACAAATTCAACAAAGAACAACAAAATGCTTTCTATGAAATCT TACATTTACCTAACTTAAACGAAGAACAACGTAACGGCTTC, SEQ ID NO. 7), B1 (GTTACGTTGTTCTTCGTTTAAGTTAGGTAAATGTAAGATTTCATAGAAA GCATTTTGTTGTTCTTGTTGAATTTGTTATCCGCGGATC, SEQ ID NO. 8) and T2 (ATCCAAAGCCTTAAAGACGATCCTTCAGTGAGCAAAGAAATTTTAGCAG AAGCTAAAAAGCTAAACGATGCTCAAGCACCAAAATAATA, SEQ ID NO. 9), B2 (TTTTGGTGCTTGAGCATCGTTTAGCTTTTTAGCTTCTGCTAAAATTTCTTT GCTCACTGAAGGATCGTCTTTAAGGCTTTGGATGAAGCC, SEQ ID NO. 10) and cloned into the BaeI-cleaved derivatives of pVSII described above. The resultant expression plasmids were designated pVS-H1-Cd, pVS-LL-Cd, pVS-PB10-Cd, pVS-PB40-Cd and pVS-PB80-Cd. Shuttle vectors containing these modified fiber genes were constructed by replacing the AgeIMfeI-fragment of the shuttle vector pKanₚHI by the AgeI-MfeI-fragments of pVS-HI-Cd, pVSLL-Cd, pVS-PB10-Cd, pVS-PB40-Cd and pVS-PB80-Cd.

### Example 4

### Expression of Ads Fiber Protein Modified With The C Domain of 20 Staphylococcus aureus Protein A

The fiber-C domain genes were assembled in the mammalian expression plasmid pVS2 and the resultant recombinant vectors were then used to direct the expression of these genes in 293T/17 cells. These expression experiments were intended to demonstrate that the designed protein chimeras could be expressed at levels comparable with that of the wild type (wt) Ad5 fiber (Fbwt) and that they possess structural and functional properties required for both the incorporation of these proteins into Ad virions and for binding to Fc-containing proteins.

293T/17 cells were transfected with the pVS-derived expression vectors using the DOTAP liposomal transfection reagent (Roche, Mannheim, Germany) according to manufacturer's protocol. Seventy-two hours posttransfection, the cells were washed with PBS, harvested, and lysed in Cell Culture Lysis Reagent (Promega, Madison, Wis.) at 10⁶ cells/ml. Cell lysates were used for enzyme-linked immunosorbent analysis (ELISA) or immunoblotting.

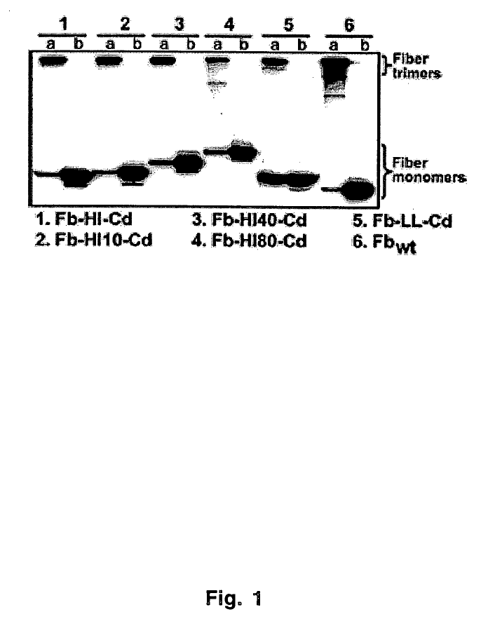

Immunoblotting of the lysates of pVS-transfected 293T/17 cells showed that the quantities of the fiber-C domain proteins were similar to the amount of the wt fiber expressed by the control plasmid (FIG. 1). A comparison of the mobilities of the chimeras in denatured and non-denatured samples clearly showed that all the newly designed proteins formed trimers upon self-association. Since trimerization of the fiber is a prerequisite of its association with the penton base protein, the results of this assay were indicative of the suitability of the fiber-C domain proteins for Ad capsid modification.

Next, Fc-binding capability of the C domain in the context of the fiber-C domain chimeras was examined. This was accomplished by an ELISA which used the lysates of fiber-C domain-expressing 293T/17 cells for a binding assay employing the Fc-G28.5 protein as bait. The wells of 96-well Nunc Immuno-plates (Fisher Scientific, Pittsburgh, Pa.) were coated overnight at 4° C. with proteins diluted in 50 mM carbonate buffer (pH 8.6) at a concentration of 5 mg/ml. The unsaturated surface of the wells was then blocked for 1 h at room temperature by the addition of 200 ml of blocking buffer (Tris-buffered saline, TBS, with 0.05% Tween 20 and 0.5% casein) to each well. The blocking buffer was replaced with 100 ml of cell lysates or Ad preparations diluted in binding buffer (TBS with 0.05% Tween 20 and 0.05% casein). The plates were incubated at room temperature for 1 h and then washed four times with washing buffer (TBS with 0.05% Tween 20). Bound fiber proteins or Ad particles were detected by incubation for 1 h at room temperature with 4D2 mAb or anti-Ad2 polyclonal antibodies, respectively. The wells were washed four times with washing buffer and then either goat antimouse immunoglobulin G or goat anti-rabbit immunoglobulin antibodies conjugated with horseradish peroxidase (HRP) (Dako Corporation, Carpinteria, Calif.) were added and the incubation was continued for 1 h. The color was developed with Sigma FAST o-phenylenediamine dihydrochloride tablet kit (Sigma, St Louis, Mo.) as recommended by the manufacturer. Color intensity was measured at 490 nm with an EL800 plate reader (Bio-Tek Instruments, Winooski, Vt.).

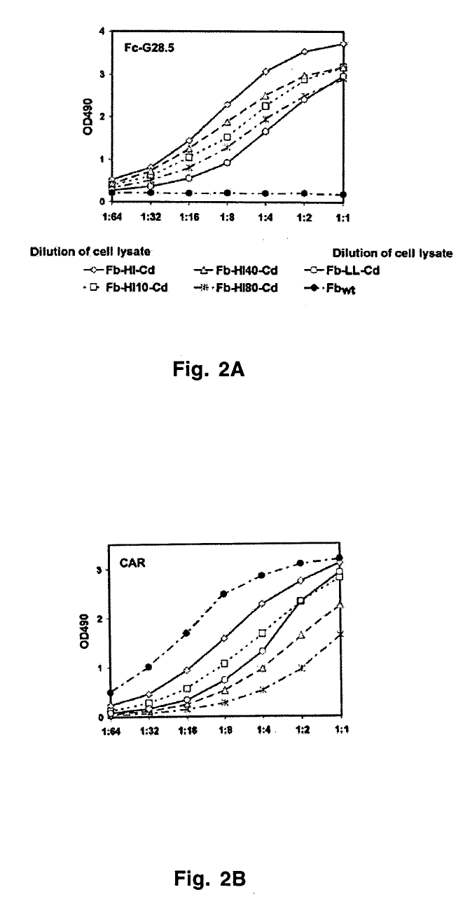

Results shown in FIG. 2A demonstrated that each of the fiber-C domain chimeras bound to the Fc domain, whereas the wild-type fiber did not bind to Fc-G28.5 even at, the highest concentration used. In addition, the interaction of the fiber-C domain proteins with CAR was examined. An ELISA employing a soluble form of CAR protein, sCAR, as the target showed that although the receptor-binding site within the modified fibers was affected by incorporation of C domain (FIG. 2B), all modified fibers largely retained the ability to bind CAR. Therefore, taken together, these experiments made it clear that despite very substantial modifications of the fiber structure, all five fiber-C domain proteins possess key functional properties that are essential for the realization of this Ad targeting scheme.

### Example 5

### Adenoviroses Containing Fiber Protein Modified With The C Domain of Staphyloccus Protein A

Recombinant Ad genomes incorporating the modified fiber genes were derived by homologous DNA recombination in Escherichia coli BJ5183 with SwaI-linearized plasmid pVL3200 essentially as described previously (Chartier et al., 1996, J Virol. 70:4805-10). pVL3200 is a derivative of pTG3602 (Chartier et al., 1996, J Virol. 70:4805-10), which contains an AdS genome deleted for the E1, E3 and the fiber gene. In place of the deleted E1 it contains a cytomegalovirus immediate early promoter-driven expression cassette comprising the firefly luciferase gene and the green fluorescent protein gene linked with an internal ribosome entry site (IRES). The designations of the pVL3200-derived Ad vectors contain the abbreviation “DR”, such as Ad5.DR-LL-Cd, to reflect the presence of a double reporter (luciferase and GFP) in their genomes.

All Ad vectors were generated by transfection of 293 cells with PacI-digested Ad rescue vectors as described previously (Krasnykh et al., 1998, J Virol. 72:1844-52). The viruses were propagated in 293 cells and purified by equilibrium centrifugation in CsCl gradients according to standard protocol (Graham & Prevec, 1995, Mol. Biotechnol. 3:207-20). Protein concentrations in viral preparations were determined by using the Dc protein assay (Bio-Rad, Hercules, CA) with purified bovine serum albumin (BSA) as a standard. Virus titers were calculated by using the formula: 1 mg of protein=4×10⁹ viral particles (vp).

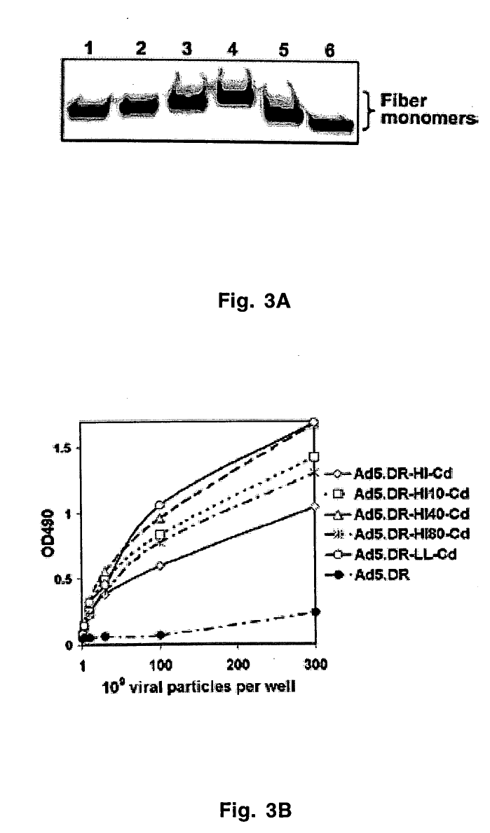

The dynamics of the infection by these vectors did not differ from those seen for a control Ad vector, Ad5.DR, incorporating wt fibers. As shown in Table 1, the titers of all six viruses were very similar. Also, as would have been predicted by the trimerization pattern of the transiently expressed fiber-C domain proteins, an immunoblot analysis of purified viruses showed efficient incorporation of these fiber chimeras into Ad capsids (FIG. 3A). Taken together, these observations suggested that the modifications of the fiber with C domain did not have any deleterious effect on the assembly of the virions.

TABLE 1
Yields of Ad DR vectors in 293 cells

| Virus | Particles per 10n cells |
|---|---|
| Ad5.DR | 1.1 × 10¹² |
| Ad5.DR-HI-Cd | 7.5 × 10¹¹ |
| Ad5.DR-HI10-Cd | 6.4 × 10¹¹ |
| Ad5.DR-HI40-Cd | 9.3 × 10¹¹ |
| Ad5.DR-HI80-Cd | 7.6 × 10¹¹ |
| Ad5.DR-LL-Cd | 8.5 × 10¹¹ |

### Example 6

### Construction of Fc-Single Chain Antibody Fusion Protein As Targeting Ligand

Having completed the modification of the Ad vectors, a complementary ligand molecule that would be capable of targeting the virus via association with its altered capsid was designed. To this end, the Fc domain of human Ig was employed as a fusion partner for a targeting single chain antibody (scFv) to generate a bifunctional “anchor-ligand” molecule. The role of the Fc domain in the present targeting scheme is two-fold. First, it is used to facilitate the expression and secretion of the targeting ligand; second, it also serves as an anchor that allows the ligand to associate with the C domain-modified Ad capsids.

The sequence encoding a fusion protein designated Fc-G28.5 comprising the secretory leader sequence, anti-CD40 single chain antibody (scFv) G28.5 (Pereboev et al., 2002, Gene Ther. 9:1189-93) tagged with the Fc domain of human immunoglobulin and six-histidine sequence (6His) was assembled within the expression cassette of the AdApt shuttle vector (Crucell, Leiden, Netherlands). The Fc-G28.5-encoding gene was placed under transcriptional control of CMV5 promoter. The genome of Ad5.Fc-G28.5 containing this cassette in place of the deleted E1 region was then generated by, homologous DNA recombination with the C1aI-linearized pTG3602 rescue vector.

To express Fc-G28.5, 6×10⁹ 293 cells were infected with Ad5.Fc-G28.5 at MOI of 100 vp/cell. The medium from the infected cells was collected at 72 h post infection and loaded onto a HiTrap rProtein A FF 5 ml column (Amersham Biosciences, Piscataway, N.J.) equilibrated with phosphate-buffered saline (PBS). After washing the column with five column volumes of PBS, bound proteins were eluted with O.1M Na-citrate, pH 3.4. To preserve the activity of the scFv, one milliliter fractions were collected into tubes with 200 ml of 1.5M Tris-HCI, pH 8.8. The collected protein was dialyzed against PBS and loaded onto a 1 ml HiTrap 6×His FF column (Amersham). After washing the column with PBS, the protein was etuted with a linear gradient of imidazole (20 to 500 mM) in PBS. The protein was collected and dialyzed against PBS. The final protein concentration was determined using the Dc protein assay (Bio-Rad) with BSA as a standard.

A total of 6.8 mg of the fusion was purified in this way upon infection of 6×10⁹ 293 cells. Analytical gel filtration chromatography of Fc-G28.5 showed that it was present in the sample in a form of a dimer, which is typical of Fc-containing proteins. Electrophoresis of the resultant preparation showed that the Fc-G28.5 ligand was more than 95% pure (data not shown) and thus suitable for subsequent vector targeting experiments.

To confirm that both components of the newly designed gene delivery system, the viral vector and the targeting ligand, were able to associate with each other, an ELISA in which Fc-G28.5 used as bait was probed with purified Ad particles. As expected, this assay showed strong binding of each of the C domain-modified vectors to the ligand, while virtually no binding was observed with the control Ad lacking C domain in the capsid (FIG. 3B). Thus, these findings proved the feasibility of the formation of targeting vector complexes and therefore rationalized subsequent cell transduction studies.

In addition to the Fc-G28.5 protein, other targeting ligands can be constructed. The design, expression and purification of the recombinant protein comprising the extracellular domain of human CAR has been reported by Dmitriev et al. (Dmitriev et al., 2000, J Virol. 74:6875-84). The expression of the 6His-tagged knob domain of Ad5 fiber in E. coli and its purification by immobilized ion metal affinity chromatography have been described previously (Krasnykh et al., 1996, J Virol. 74:6875-84). All chromatographic separations were performed utilizing the AKTApurifier system on prepacked columns from Amersham Pharmacia Biotech Inc. (Piscataway, N.J.).

Recombinant protein Fc-CD40L, which consists of a genetic fusion of the DNA encoding the human tumor necrosis factor (TNF)-like domain of human CD40 Ligand sequence at its amino terminus to the hinge region of the Fc domain of human IgGg1, was expressed in marine NS/0 cells and purified as previously described (Lo et al., 1998, Protein Eng. 11:495-500).

### Example 7

### Preliminary Assessment of Gene Transfer Properties of Ad::ligand Targeting Complexes

A comparison of the gene delivery characteristics of the Ad::Fc-G28.5 complexes was done by means of a transduction experiment employing 293.CD40 cells as the target. Since all the Ad vectors used in these studies contained fibers with functional CAR binding sites, CAR on the surface of the target cells were blocked with knob protein (Krasnykh et al., J Virol. 1996 October; 70(10):6839-46) in order to discriminate between CAR-mediated cell entry versus that which was, expected to result from the attachment of the targeting complexes to CD40. Prior to infection with the modified Ad vectors, the cells were preincubated with either medium alone, medium containing recombinant Ads fiber knob protein, or medium containing the knob and Fc-G28.5 ligand. Ad vectors incorporating wt fibers, and parental 293 cells that do not express any detectable CD40 were employed as negative controls.

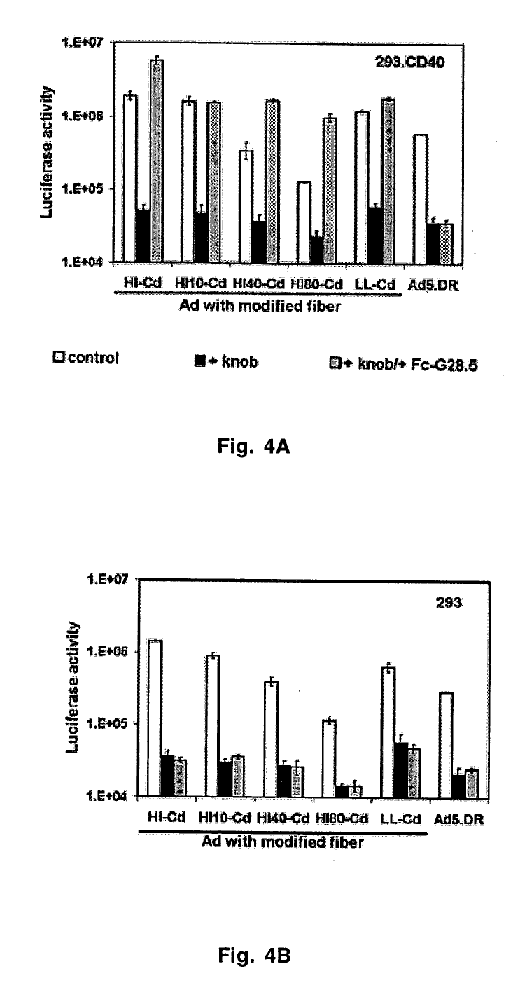

This experiment showed that all C domain-modified Ad were able to employ the Fc-G28.5 ligand for CD40-mediated infection, with no significant variations between the vectors (FIG. 4). These data obviated the need to continue the work with all five modified vectors. Therefore, Ad5.DR-HI10-Cd, Ad5.DR-H40-Cd, and Ad5.DR-LL-Cd were chosen for the following experiments, as these constructs represented two different Ad fiber modification approaches: the redesign of the HI loop and the carboxy terminus of the protein.

### Example 8

### Preparation And Characterization of Preformed Ad::ligand Complexes

Complexes of Ad with Fc-containing targeting ligands were generated during purification of viruses from infected 293 cells. Briefly, 293 cells were infected with adenoviruses at a multiplicity of infection (MOI) of 300 vp/cell. Cells were harvested at 55 h post-infection and resuspended in 2% FBS/DMEM. Viruses were released from the cells by three freeze-thaw cycles, and the cell debris was removed by centrifugation. The supernatant was layered onto a preformed step gradient of CsCl and centrifuged at 25,000 rpm for 3 h at 4° C. Banded viruses were collected, mixed with Fc-G28.5 or Fc-CD4OL proteins at a concentration of 30 mg/ml and incubated for 30 min at room temperature. All the C domain anchoring sites within the virions are expected to be occupied by the targeting ligands under high ligand-to-virus ration. Vector complexes were purified from unbound proteins by equilibrium centrifugation in CsCl gradients, dialyzed (10 mM Tris-HCl, pH8.0, 50 mM NaCl, 2 mM MgCl₂, 10% glycerol) and stored at −80° C. until use.

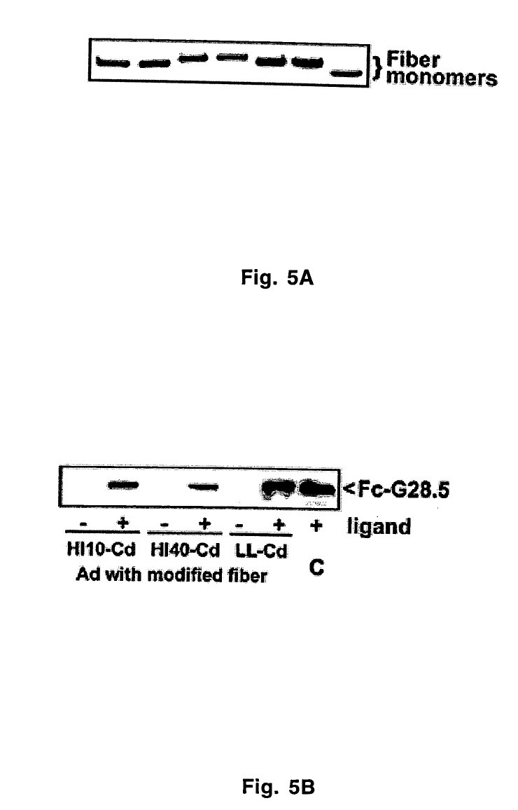

Each of the three viruses, Ad5.DR-HI10-Cd, Ad5.DR-H140Cd, and Ad5.DR-LL-Cd, was mixed and incubated with the targeting Fc-scFv ligand as described above. The efficiency of association of the ligand with each of the viruses was examined in an immunoblot assay using a Penta-His mAb that binds to the 6His tag present in the ligand molecule. This analysis showed that Fc-G28.5 protein bound most efficiently to Ad5.DR-LL-Cd, while the amount of the ligand found in preparation of AdS.DRHI10-Cd and Ad5.DR-H140-Cd was lower (FIG. 5).

### Example 9

### Transduction Properties of The Preformed Ad::Ligand Complexes On Established Cell Lines

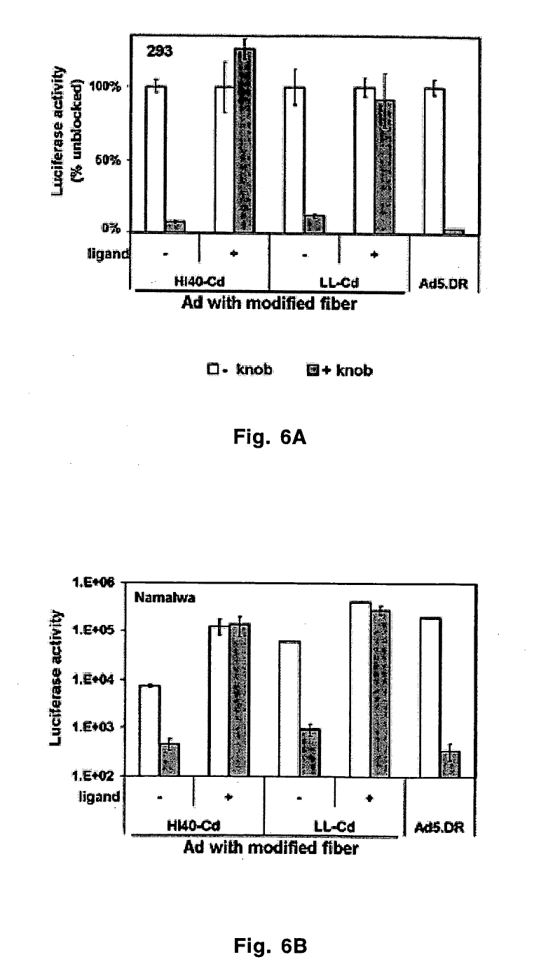

The receptor specificity of the resultant vector complexes was assessed by employing them to infect two different cell targets. First, these complexes were used to transduce 293 cells, which are CAR-positive but do not express any detectable CD40. The main purpose of this experiment was to test whether the association of Ad vectors with the ligand affected the viruses' ability to hind CAR. Ad5 fiber knob protein was added to duplicate samples to block CAR receptors present of the cells. Predictably, when used without a ligand, each of the viruses was capable of using CAR for cell entry, as evidenced by efficient inhibition by the knob protein. In contrast, the infectivity of Ad::Fc-G28.5 vector complexes was not affected by the presence of the knob (FIG. 6A).

These vectors were then employed for infection of Namalwa human lymphoblastoid cells, which are CAR-positive and naturally express CD40. As seen in FIG. 6B, the vector complexes clearly outperformed the relevant untargeted Ad, with the difference in the infection efficiencies being in the range of an order of magnitude for each vector. Importantly, this augmentation of infectivity was entirely due to targeting of the vectors to CD40, as the addition of the fiber knob protein had no effect on gene transfer. Of special note, Ad5.DR-HI10-Cd demonstrated an infection profile which was very similar to that of Ad5.DR-HI40-Cd (not shown).

The CD40-dependence of the infection by the targeted complexes was further confirmed by transducing Namalwa cells with Ad5.DR-LL-Cd::Fc-G28.5 in the presence of various concentrations of free ligand. This resulted in a Fc-G28.5 concentration-dependent inhibition of transduction, which unambiguously demonstrated the direct involvement of CD40 in the cell entry pathway used by the ligand-containing vector complex (FIG. 7). As expected, the infectivity of the Ad5.DR vector, which contains wild type fibers and is thus unable to associate with Fc-G28.5, was not affected by the addition of the free ligand.

### Example 10

### In Vitro Transduction of Primary Human Dendritic Cells With The CD40-Targeted Vectors

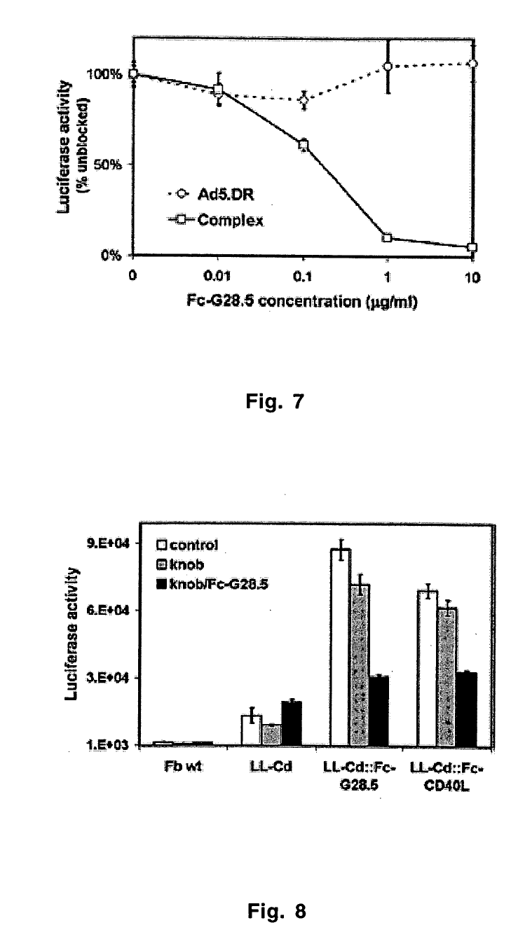

An additional test of the cell transduction ability of the Ad5.DR-LL-Cd::Fc-G28.5 vector was done using human dendritic cells (DCs) as targets. These DCs were derived from CD14-positive monocytes isolated from human peripheral blood. For the purpose of comparison, a similarly prepared vector complex containing the CD40-binding domain of human CD40 Ligand, CD4OL, fused with Fc was also employed. This experiment demonstrated that, when complexed with either of the two targeting ligands, the C domain modified vector was able to deliver the reporter gene to dendritic cells 28- to 35-fold more efficiently than the control unmodified vector, Ad5.DR (FIG. 8).

In line with previous reports of poor expression of CAR and elevated levels of CD40 in dendritic cells, the use of the Ad5 fiber knob and scFVG2s.5 as inhibitors of infection revealed that the CD40-mediated component of overall gene transfer by the targeted vectors was higher than that involving CAR, which was observed for untargeted Ad. On another note, the scFV_{G28.5} constituent of the targeting protein was more efficient in directing the vector complex to dendritic cells than was the natural ligand of CD40, CD40L, thus further supporting the choice of scFvs as targeting moieties for Ad.

### Example 11

### Construction of Targeted Adenoviral Vector For Selective Expression of Tumor-Specific Antigen In Dendritic Cells

The following example describes the construction of targeted adenoviral vector for selective expression of tumor-specific antigen in dendritic cells. The cloning procedure involves the following steps:

generating an Ad shuttle vector containing an expression cassette incorporating genes encoding a tumor-specific antigen and a targeting ligand;

incorporating the dual expression cassettes into a fiber gene-deleted, green fluorescent protein-expressing Ad genome;

cloning of mammalian expression plasmids incorporating genes encoding for Ad fibers modified with the C-domain of S. aureus protein A (CdpA);

transient expression of the fiber-CdpA proteins in 293T cells for structural integrity assessment;

transferring the fiber-CdpA-encoding genes into an Ad fiber shuttle vector;

transferring the fiber-CdpA-encoding genes from the Ad fiber shuttle vectors ino the fiber gene-deleted Ad genome expressing the tumor-specific antigen and the targeting ligand; and

rescue and amplification of the viruses of interest.

Adenoviral shuttle vector containing an expression cassette incorporating genes encoding a targeting ligand and a tumorspecific antigen is constructed as follows. The vector is designed using the Ad shuttle plasmid which contains an expression cassette driven by the strong cytomegalovirus promoter. First, the expression cassette within the plasmid is duplicated and multiple cloning sites within one of the two cassettes is replaced with a synthetic DNA sequence containing a set of alternative cloning sites. The plasmid containing this double cassette will allow the cloning of transgenes into either of the two polylinker sequences. DNA sequence encoding a tumor-specific antigen, such as the cDNA of prostate-specific membrane antigen, is cloned into one of the cassettes. Subsequently, sequence encoding fusion proteins comprising either the soluble form of CD40L (sCD40L) or anti-CD40 scFv G28.5 tagged with the Fc domain of human immunoglobulin is cloned into the other cassette. This targeting ligand is designed to target Ad vectors incorporating within their capsids C-domain of S. aureus protein A. All targeting ligand-encoding sequences described here are designed by the “sticky end” PCR technique.

The dual expression cassette is then incorporated into a fiber gene-deleted, green fluorescent protein-expressing Ad genome. First, the E3 region of an Ad5 genome contained in the Ad rescue vector pVK is replaced with an expression cassette containing the green fluorescent protein (GFP) gene. This is followed by incorporating the dual expression cassettes constructed above in place of the E1 regions of the Ad genome contained in the resultant rescue plasmid. Transfer of all transgenes into the Ad genome is done by the method of homologous DNA recombination in bacteria originally described by Chartier et al. (Chartier et al., 1996, J Virol. 70:4805-10).

To construct mammalian expression plasmid incorporating gene encoding Ad fiber modified with the C-domain of S. aureus protein A (CdpA), CdpA can be genetically fused with either the carboxy terminus of the previously described Ad5 fiber:T4 fibritin protein chimera (Krasnykh et al., 2001, J. Virol. 4176-4183), or the HI loop of the Ad5 fiber knob domain. Sequence encoding the C domain is cloned into the BaeI-cleaved mammalian expression vectors pVS.F_{cBae1} or pVS.F_{FBae}I, which contain the genes for the fiber and fiber:fibritin, respectively. As a result of this cloning step, the open reading frames of each of the two carrier proteins will be fused with that of the C domain.

The fiber-fibritin chimera is employed as an alternative strategy to generate the fiber-C domain chimeric gene. The fiber-fibritin protein was designed so that the structure of the domain providing for tcimerization of the chimera (fibritin) is not affected by incorporation of heterologous peptides/polypeptides within the protein, thereby dramatically increasing the odds of obtaining stable derivatives of this “backbone” molecule. This strategy of fiber replacement has been described in a recent paper (Krasnykh et al., 2001, J. Virol. 4176-4183).

The expression plasmids of the pVS series described above can be used to direct production of the C domain-modified fibers in mammalian cells. For this 293T cells are transfected with each of the pVS vectors and the expression of the fiber-C domain proteins is assessed 48 hrs later by lysing the cells and analyzing their lysates by Western blot with anti-fiber tail mAb 4D2. As the trimeric structure of Ad fiber is a prerequisite for its successful incorporation into an Ad virion, this assay will allow us to identify those fiber-C domain species that can be employed for the Ad targeting disclosed herein.

The expression plasmids of the pVS series are designed to be “compatible” with the fiber shuttle vectors of the pKan series to insert modified fiber genes into Ad genomes. Those fiber-C domain genes whose products have successfully passed the trimerization test are cloned into the pKan vectors in a simple subcloning step utilizing the same pair of restriction enzymes (MfeI and AgeI) for all constructs to be made.

The genes encoding the newly designed fiber-C domain proteins are then incorporated into the Ad rescue vectors constructed above by homologous DNA recombination in bacteria. The fiber-C domain genes are incorporated into Ad genomes containing the genes for Fc-ligands, whereas zipper-fiber genes are inserted into the genomes incorporating zipper-Fc-ligand genes. Consequently, the design of Ad genomes of interest is completed and the viruses of interest are rescued and amplified in 293 cells.

### Example 12

### Induction of Dendritic Cells Maturation Upon CD40-Mediated Infection.

The following example examines the effects of vector targeting to CD40 on the phenotype of dendritic cells. It is expected that not only can CD40-targeted vectors deliver antigen-expressing genes to dendritic cells in a more efficient manner, but also that they are able to trigger maturation and activation of dendritic cells and thus launch the generation of an immune response. In this regard, it is known that activated dendritic cells have a characteristic phenotype, which can be shown by flow cytometry and also confirmed functionally by examination of the cytokines they secrete and the cytokines they induce T cells to secrete. In addition, activation of naive CD4⁺ T cells is a hallmark of dendritic cell function. These functions can be examined by various immunologic assays described below.

Day 5 dendritic cells (DCs) are transduced with CD40-targeted Ad vectors or control Ad lacking targeting capacity. Twenty-four hours later aliquots of dendritic cells are subjected to fluorescence-activated cell sorting (FACS) for analysis of CD40, CD54, CD80, CD86 (T cell co-stimulatory markers), CD83 (DC maturation marker), CCR7 (lymph node homing marker) and CCR6 (immature DC marker) expression. It is expected that, targeted Ad vectors will induce DC maturation/activation significantly better than control Ad, as will be evidenced by increased expression of CD40, CD54, CD80, CD83 and CD86. CCR6 expression is expected to be downregulated, while the mature DC marker CCR7 is expected to be expressed at an elevated level. CCR7 is associated with lymph node homing, and thus increased CCR7 expression can improve in vivo immunogenicity of transduced DCs.

Dendritic cell function can be assessed by two independent means: i) analysis of secreted DC products and ii) analysis of effects on T cell function. Myeloid DCs secrete IL-12 upon activation to induce a strong Thl polarized immune response dominated by T cell interferon-g. IL- 10 is also induced and this can reduce induced interferon-g. Day 5 dendritic cells are transduced with adenovirus as above and IL- 12 and IL-10 are measured in the supernatant 24 hours later by ELISA (R&D Systems). Controls include non-targeted vector and “no treatment” as negative controls. Lipopolysaccharide from E. coli LPS is used at 100 ng/ml as a positive control. CD40-targeted Ad vectors are expected to induce DC maturation/activation significantly better than those not targeted to CD40, as will be evidenced by an increased capacity of DCs to secrete IL-12. IL-10 may also be induced, but not at higher levels than in control samples.

T cells activated by myeloid dendritic cells secrete significant amounts of interferon-g and IL-2, with little IL-4 and no IL-1 0. T cells are activated by incubation with Ad-transduced day 5 dendritic cells 24 hours post transduction. Induced cytokines can be assessed at single cell level by in situ cytokine detection assay as previously described (Zou et al., 2000, J Immunol. 165:4388-96 and Zou et al., 2001, Nat Med. 7:1339-46), and confirmed by ELISA of supernatants. T cell activation are confirmed by proliferation in an allogeneic mixed lymphocyte reaction (MLR).

Here, naive CD4⁺ CD62L⁺ CD45RO⁻ CD4⁺ T cells are isolated using beads (Miltenyi) as described (Zou et al., 2000, J Immunol. 165:4388-96 and Zou et al., 2001, Nat Med. 7:1339-46), and MTT dye uptake and total cell numbers are measured 3 days later.

Tumor-specific CTLs are thought to be pivotal effectors in specific immunity. CTL-inducing capacity of dendritic cells transduced with targeted Ad vectors can be examined by a generic approach and a tumor-specific approach. For the generic approach, interferon-g⁺ CD8⁺ T cells, which are accepted surrogates of CD8⁺ CTLs, can be detected by flow cytometry as described (Zou et al., 2000). Allogeneic CD8⁺ T cells are incubated with Ad-transduced dendritic cells and interferon-g⁺ CD8⁺ T cells can be detected by flow cytometry 3 days later.

Prostate-specific membrane antigen (PSMA)-specific immunity can be examined using peripheral blood CD3⁺ total T cells induced to proliferate with 2 HLA A2-restricted peptides. Tetramers for these peptides can be synthesized as previously described (Altman et al., 1996). Influenza matrix ₅₈₋₆₆ peptide (which binds to HLA A2) is used as a control. Tetramer complexes can be combined with PE, or allophycocyanin (APC)-labeled streptavidin, and tetramer⁺ cells are analyzed by FACS. These studies can be confirmed with cytotoxicity assays using [⁵¹Cr]-labeled T2 cell lines (ATCC) pulsed with or without the HLA-A2-restricited PSMA peptides as targets in standard [⁵¹Cr] release assay. Negative controls include T2 cells pulsed with influenza matrix ₅₈₋₆₆ peptide and T2 cells with no peptide. Consistent with the mature/activated phenotype of Ad transduced dendritic cells, it is expected that they will activate a higher level of T cell roliferation and induce significant levels of interferon-g and IL-2 production by T cells. As CD40 ligation enhances CTL activity, it is also expected that dendritic cells activated by the CD40-targeted Ad will exhibit better CTL activity compared to dendritic cells transduced with non-targeted Ad.

### Example 13

### The Ability of CD-40-Targeted Ad Vectors To Induce Maturation And Migration of Human Dendritic Cells

Dendritic cells naturally present in human skin mimic the anticipated use of DC-targeted Ad vectors for immunization via intradermal injection. The goal of following studies is to show that targeting of Ad vectors to dendritic cells via the CD40-pathway allows the vectors to find and selectively transduce their cell targets (DCs) in a complex context of a real human tissue.

Skin explants cultured with the epidermal side up on filter-covered grids over a period of 24 hours are injected with CD40-targeted Ad vectors or plain medium. The explants are placed in culture medium (floating with the epidermal side up) in a 48-well culture plate and further incubated before migrating dendritic cells are harvested. Subsequent studies including cytometry, immunohistochemistry and MLR performed according to protocols well known in the art.

### Example 14

### Incorporation of Novel Fc-Binding Domain into Adenoviral Fiber Protein Results in Enhanced Stability of Targeting Complexes Formed with Fc-Containing Ligands

Adenovirus serotype 5 (Ad5) has shown potential as a gene delivery vehicle for numerous gene therapy applications. In one targeting scheme, the receptor-selective affinity of immunoglobulin G (IgG) molecules has been employed to retarget AdS via the incorporation of Fc-binding domains on the Ad5 capsid, through which tropism alteration is achieved via Ad-IgG complexes. This system provides for a flexible, modular approach to targeting cancer cells by any human-Fc containing ligands. The use of the C-domain (Cd) of protein A from S. aureus for this targeting scheme (Ad.Cd) has been previously documented, however, this domain also has a characterized high affinity for the Fab regions of IgG. Because binding between these two domains is non-competitive, for in vivo utility, the ability to bind both domains could potentially lead to multiple Fc/Fab containing ligands forming complexes with these modified vectors, thus undermining the specificity of the targeting ligand employed for the desired gene therapy application. To circumvent this problem, a novel Fc-binding peptide, the Zc domain has been engineered, based on the literature of well-known non-Fab-binding, Fc-binding domains. With Ad.Zc, the ability of the vector to bind the Fab regions of IgG molecules has been abolished, via site-directed mutagenesis of a single glycine to alanine substitution in the Fc-binding peptide. With this structural modification to our previous vector, the ability of Ad.Zc::Fc-ligand complexes to efficiently transduce cells in a CAR-independent manner has been demonstrated. Furthermore, this new variant effectively retains the interaction with human Fc-containing targeting ligands, when introduced into environments induced with competing immunoglobulins. Hence, with Ad.Zc, a fundamental improvement to the previously reported two-component targeting approach has been shown, enhancing this technology for in vivo gene therapy applications.

Therapeutic gene delivery has emerged as a promising means of combating cellular defects at the molecular level. Because the effectiveness of gene therapy is predicated upon the transfer of therapeutic agents to target cells, gene delivery vehicles capable of efficient and specific gene transfer are mandated. Of currently used vector systems, human adenovirus serotype 5 (Ad5) has shown potential as a delivery vehicle for various gene therapy applications. Efficient gene transfer in vivo, and a relative ease in development and production of modified vectors, has added to the attractiveness of AdS as a gene therapy vector (see, e.g., Glasgow et al., (2004) Curr Gene Ther 4: 1-14).

Ad5, a species C member of the family Adenoviridae, is a non-enveloped, icosahedral virus housing a 36 kb, double stranded DNA genome. Ad5 demonstrates efficient gene transfer to both dividing and non-dividing cells, and employs a two-step mechanism for viral docking and subsequent entry into target cells. The globular knob domain, located at the distal end of the fiber homo-trimers extending from twelve capsid vertices, binds to its native receptor, coxsackie and adenovirus receptor (CAR) (see, e.g., Bergelson et al. (1997) Science 275: 1320-3 and Henry et al. (1994) J Virol 68: 5239-46). Following this initial knob-CAR interaction, a subsequent interaction takes place between the RGD motif of the Ad5 penton base, and the cellular integrins αᵥβ₃ and a αᵥβ₅ (see, e.g., Bai et al. (1994) J Virol 68: 5925-32 and Wickham et al. (1993) Cell 73: 309-19). This secondary step initiates viral endocytosis within a clathrin-coated vesicle, with subsequent viral release into the cytoplasm resulting in nuclear translocation and viral replication. However, in a variety of gene therapy contexts, the paucity of CAR on target cells, coupled with its widespread distribution on non-target cells, has proven deleterious to the utility of Ad5 as a gene therapy vector (see, e.g., Hemminki & Alvarez (2002) BioDrugs 16: 77-87).

To circumvent the limitations associated with native Ad5 tropism, modifications to the Ad5 knob have shown promise in targeting these vectors to non-native receptors. By exploiting available cell surface markers on target cells, modified Ad vectors can ensure gene delivery to only those target cells of interest, via CAR-independent retargeting (see, e.g., Glasgow et al., (2004) Curr Gene Ther 4: 1-14). Many strategies have been employed in modifying Ad5 capsid proteins providing moderate improvement in transductional retargeting. Two locales exploited for such engineering include the C-terminus of Ad5 knob (see, e.g., Bouri et al. (1999) Hum Gene Ther 10: 1633-40 and Wickham et al. (1997) J Virol 71: 8221-9) and the HI loop (see, e.g., Krasnykh et al. (1998) J Virol 72: 1844-52 and Dmitriev et al. (1998) J Virol 72: 9706-13), a flexible peptide region protruding from the knob domain. Ad5 vectors with short peptides genetically incorporated at these sites have been found to maintain structural integrity while offering CAR-independent tropism. Although elegant, this strategy is limited by the size and structure of peptides that can be incorporated at these locales (see, e.g., Bouri et al. (1999) Hum Gene Ther 10: 1633-40 and Dmitriev et al. (1998) J Virol 72: 9706-13).

Antibodies (Ab) of the immunoglobulin class G (IgG) are natural targeting molecules exhibiting high specificity for the particular antigen against which they are directed, making these molecules an attractive candidate for Ad vector retargeting. However, their relatively large size has hindered any attempt to develop such a technology in an Ad5 vector, leading to alternate strategies in utilizing the targeting capabilities of the IgGs. Of these, genetic incorporation of a peptide that binds the Fc domain, common to all IgG molecules, has shown potential in retargeting via vector-IgG complexes, and by viral complexes with proteins containing single chain antibodies (scFv) fused with the Fc domain (Fc-scFv). Specifically, the various Fc binding domains of Staphylococcus aureus protein A (SpA) have been employed for this targeting strategy (see, e.g, Volpers et al. (2003) J Virol 77: 2093-104 and Henning et al. (2002) Hum Gene Ther 13: 1427-39). Applicants have previously shown that a vector with the Fc and Fab binding C-domain (Cd) of SpA genetically fused to the C-terminus of Ad5 knob, Ad.Cd (previously known as Ad5.DR-LL-Cd), effectively retargeted vectors in vitro via Fc-containing fusion proteins (see, e.g., Korokhov et al. (2003) J Virol 77: 12931-40). Further, this study showed that pre-formed Ad5.Cd::Fc-scFv complexes maintained their stability upon purification and storage, and effectively retained the ability to infect cells via CAR-independent mediation. However, we hypothesized that Ad.Cd complexed with any Fc-containing targeting ligand, e.g., a whole IgG molecule or the Fc-scFv, would prove to be unstable when placed in environments with competing IgGs, which might displace the targeting ligand for more favorable Cd-Fc interactions, or sterically hinder the targeting ligand from recognizing the desired receptor; such as would be the case in in vivo applications. To a certain extent, this could be accounted for by the ability of Cd to bind the Fab regions common to all IgG molecules, in addition to the Fc domain.

To circumvent this potential problem, Applicants have engineered a novel IgG-binding ligand, the Zc, by modification of the Fc binding domain previously employed for this targeting schema. Based on non-Fab-binding, Fc binding domains (see, e.g., Jansson et al., (1998) FEMS Immunol Med Microbiol 20: 69-78), Applicants have abolished the ability of the C-domain to bind the Fab regions of IgG molecules, and have genetically incorporated the domain at the C-terminus of the Ad5 knob, creating Ad.Zc. Further, Applicants have characterized the ability of these vectors to transfer genes in vitro, via pre-formed complexes with IgG and Fc-scFv. Most importantly, we have shown that only Ad.Zc pre-complexed with Fc-containing ligands, retains its targeting abilities when introduced into environments with competing immunoglobulins. Herein, Applicants' offer a fundamental and critical improvement to Applicants' previous Fc-binding adenoviral technology, optimizing this targeting schema for further application.

Design, Expression, and Characterization of Fibers with C-terminal Zc Domain.

To generate a mutant form of C domain (Cd) with reduced Fab-binding, the glycine residue at position 29 of C-domain was replaced with alanine to generate the Zc-domain. For preliminary experiments the Cd and Zc open reading frames were incorporated via a (GGGGS)₃ (SEQ ID NO. 1) linker (LL) at the C-terminus of fiber fibritin, to ensure adequate yield of the modified proteins in 293T/17 cells (see, e.g., Krasnykh et al., J Virol 75: 4176-83). Fusion protein genes were assembled in the mammalian expression vector pVS2 (see, e.g., Korokhov et al. (2003) J Virol 77: 12931-40). Transiently expressed chimeric proteins containing the C-terminal Cd or Zc binding domain were expressed in 293T/1 7 cells for preliminary Fc/Fab binding experiments.

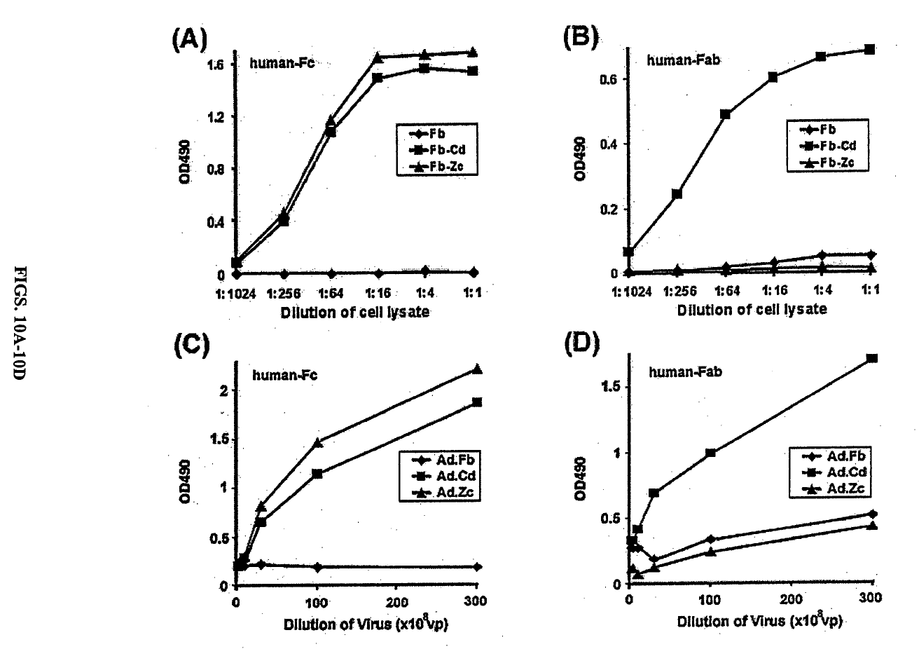

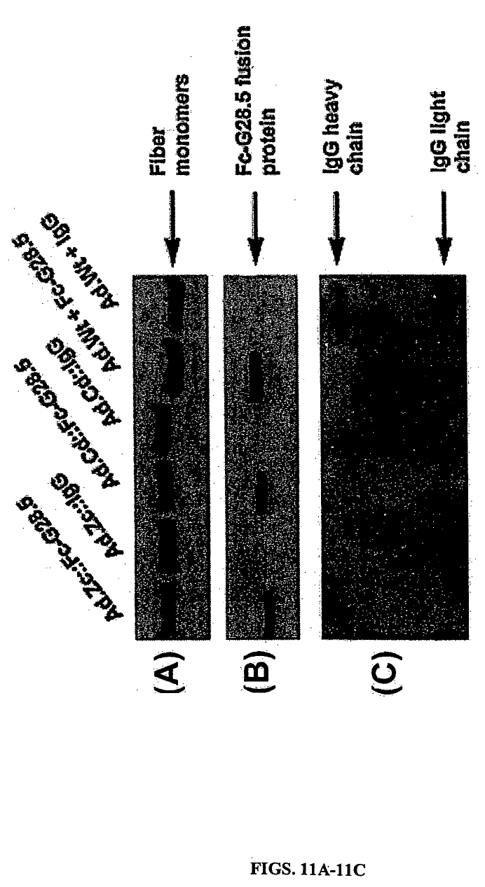

To determine the Fc/Fab binding characteristics for both chimeric variants, an ELISA employing either human-Fc or human-Fab as a bait protein, was conducted using lysates of pVSZc/Cd-transfected 293T/17 cells expressing either Zc or Cd fusion proteins (FIGS. 10A and 10B). This assay demonstrated that each of the chimeras bound to the human-Fc domain, while, predictably, the Zc variant showed minimal affinity for human-Fab . As expected, the negative control wild-type Ad5 fiber displayed no binding affinity for either protein.

Derivation of Ad Vectors containing Zc-Modified Fibers.

A fiber shuttle vector containing the Zc-modified Ad5 fiber gene was constructed and recombined with an Ad5 genome containing the gene encoding green fluorescent protein (GFP) under the control of the CMV promoter in the E1 region. In this capacity, GFP would serve as a reporter for gene transfer analysis. Recombinant genomes were isolated, purified, and used for transfection of 293 cells. After an initial viral rescue, the vectors were propagated, CsCl purified, and their titers were determined. According to immunoblot analysis of purified viruses the Zc-modification to the fibers had no adverse affects on their assembly with the Ad5 capsid (FIG. 11A).

Applicants then sought to characterize, by ELISA, the human-Fc/Fab binding characteristics of the recombinant Ad vectors with the modified fiber proteins. As expected, the ELISA displayed that both Ad.Cd (previously known as Ad5.DR-LL-CD, see Korokhov et al. (2003) J Virol 77: 12931-40) and Ad.Zc displayed affinity for the human Fc protein (FIG. 10C), while only Ad.Cd retained the ability to bind human Fab (FIG. 10D).

Preparation and Characterization of Pre-Fformed Ad-Fc Ligand Complexes.

To determine the targeting efficiency of Ad::Fc-ligand pre-formed complexes, Applicants first prepared purified Ad.Zc and Ad.Cd vectors complexed with a Fc-scFv fusion protein against human CD40 (Fc-G28.5), or a murine monoclonal antibody (mAb) against human CD40 (G28.5). The vectors were propagated, incubated with targeting ligands (ligand/virus ratio—1,800:1), and purified. To assess the efficiency of association of the ligands with each of the viruses, a western blot analysis was performed on the purified complexes. Fc-scFv, which contains a 6His tag, was probed with a Penta-His mAb, while G28.5 was probed with rabbit anti-mouse polyclonal antibodies. The results showed that Fc-scFv was efficiently complexed with both Ad.Cd and Ad.Zc (FIG. 11B); however, there was no detectable amount of G28.5 antibody in any viral preps (FIG. 11C). This prompted Applicants to further investigate the stability of the interaction of Cd or Zc with the Fc domain of murine and human IgG.

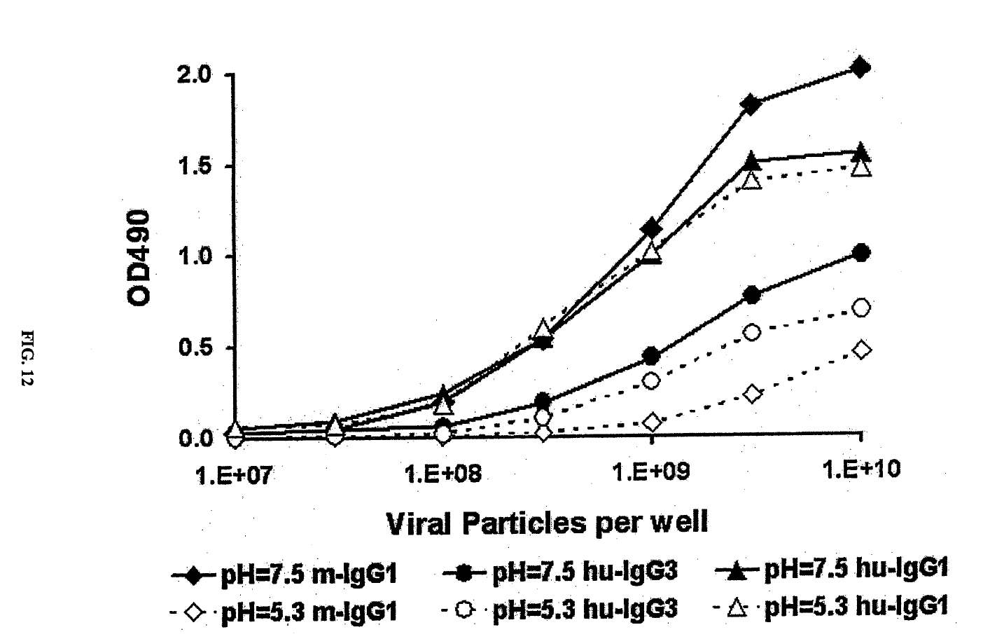

The stability of Ad.Cd::IgG complexes was examined by comparing viral binding of human and murine IgG molecules while varying the pH of buffer in which the complexes were maintained. To this end, an ELISA experiment was performed by adsorbing either of two isotypes of human IgG, human IgG1 and human IgG3, or a murine counterpart, mouse IgG1 to the wells of the ELISA plate, and incubating with Ad.Cd at various pHs. In full agreement with the binding characteristics of protein A, Ad.Cd displayed high affinity for human IgG1 in both of the pH-variant environments (FIG. 12) and no affinity for human IgG3 antibodies. Although Ad.Cd::mouse-IgG1 forms stable complexes at physiological pH, the binding affinity between the two components was not retained when the pH was significantly lowered to 5.3 (FIG. 12). These findings correlate with the lack of G28.5 (IgG), a murine IgG1 against CD40, observed in our previous immunoblot analysis of the targeting ligand in Ad-G28.5 pre-formed complexes (FIG. 11C).

Gene Transfer Analysis of Pre-formed Ad-ligand Complexes.

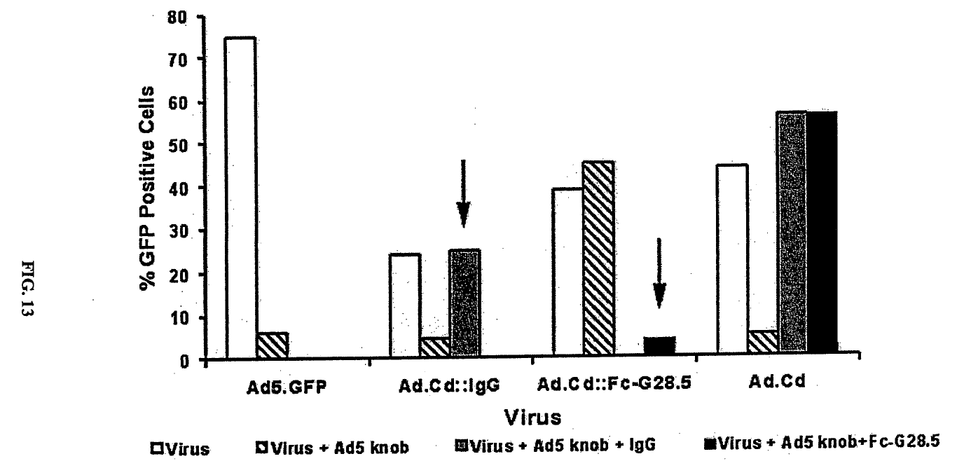

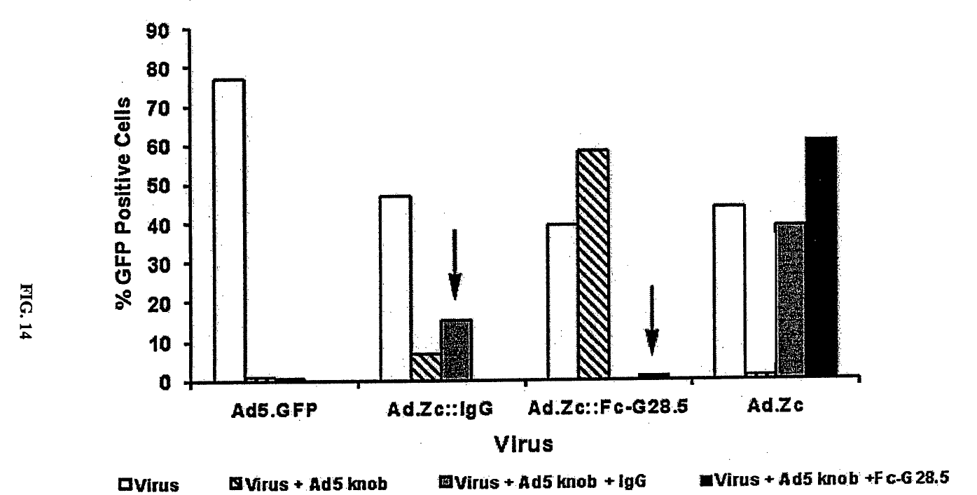

To further examine the gene transfer efficiency of CD40 targeted Ad.Cd and Ad.Zc via complex with Fc-G28.5 (Fc-scFv) or mAb G28.5, the vectors were used to transduce 293 cells expressing human CD40 (293.CD40). Because the modifications to these vectors did not alter native knob tropism, 293.CD40 cells were pre-incubated with Ad5 knob to exclude gene transfer due to knob-CAR interaction (see, e.g., Krasnykh et al. (1996) J Virol 70: 6839-46). According to our results, CAR-independent, CD40 mediated gene transfer of 293.CD40 cells was achieved with both Ad.Cd::Fc-scFv (FIG. 13) and Ad.Zc::Fc-scFv (FIG. 14) complexes. Additionally, the cells were pre-incubated with the free targeting ligand employed in the viral complex before infection, e.g., cells infected with Ad.Cd::G28.5, were pre-incubated with the G28.5 antibody. This would lead to inhibition or augmentation of gene transfer, depending on the extent to which ligand molecules occupied potential capsid locales. If the viral capsid contained no available binding sites due to complete ligand incorporation during Ad-ligand incubation, the free targeting ligand would serve as a competitive inhibitor of CD40 receptors. Alternatively, gene transfer augmentation would demonstrate that free Fc binding sites remained on the capsid fibers, suggesting that pre-formed Ad-ligand complexes did not maintain interaction with the targeting ligand at every available capsid locales.

The gene transfer data for Ad.Cd are displayed in FIG. 13, and for Ad.Zc in FIG. 14, with infections done at a multiplicity of infection (MOI) of 40 vp/cell. For both viruses, pre-incubation of cells with MAb G28.5 and subsequent infection with Ad.Cd/Zc::G28.5 led to augmentation of gene transfer, further confirming that the pre-formed Ad::IgG complexes were inefficiently maintained during preparation and storage. In contrast, infection of Ad.Cd/Zc::Fc-G28.5 (Ad.Cd/Zc::Fc-scFv) displayed inhibition of gene transfer, as the free targeting ligand competitively inhibited 293.CD40 cell transduction with Ad-Fc-scFv complexes. Of note, there was a significantly greater augmentation of gene transfer in cells pre-incubated with knob and IgG, when infected with Ad.Cd::IgG compared to Ad.Zc::IgG. This observation could be explained by the ability of pre-formed Ad.Cd::IgG complexes to bind the Fab regions of IgG molecules present on the cell surface, if the Fab-binding site remained free on the C-terminus of the knob protein. The Fab region would provide an additional binding locale for the virus, increasing the frequency of Ad transduction via interaction with cell surface IgG ligands. These results are further corroborated by the infection profile of uncomplexed Ad.Cd versus Ad.Zc (FIGS. 13 and 14) in cells subjected to the same experimental conditions, which demonstrate that uncomplexed Ad.Cd vectors transduce cells pre-incubated with IgG more efficiently than uncomplexed Ad.Zc.

Gene Transfer Analysis of Pre-Complexed Ad::Fc-scFv after Incubation with Competing Human-IgG1.

After characterizing the gene transfer efficiency of the Ad::ligand pre-formed complexes, Applicants then sought to determine the stability of these complexes, and their ability to transfer genes, after incubation with another Fc-containing ligand. This experiment would mimic an environment such as the systemic circulation, in which many competing Ig molecules would be present. To this end, Applicants employed the human-IgG1 molecules, which are representative of the majority of IgG in human serum and have high affinity for protein A. In addition, upon examining the data obtained from the preliminary gene transfer experiments, we sought to endeavor this experiment for the Ad vectors pre-complexed with the Fc-scFv fusion protein only, because these variants demonstrated higher stability than that of Ad::IgG.

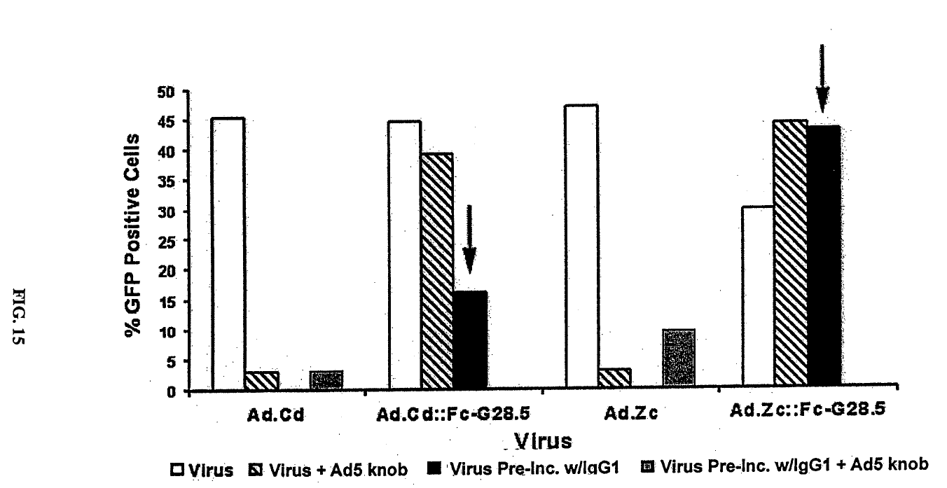

Working dilutions (2×10⁷ vp) of viral infection media were incubated with a high excess (30 μg) of human IgG1 for 30 minutes at room temperature, and the gene transfer data of 293.CD40 cells was obtained via FACS analysis (FIG. 15). Incubation of pre-complexed Ad.Cd::Fc-G28.5 (Ad.Cd::Fc-scFv) with human IgG1 resulted in the inhibition of gene transfer, subsequently rendering the virus untargeted. Moreover, the inhibitory effect on gene transfer observed in Ad.Cd::Fc-scFv incubated with human-IgG1 on cell lines not blocked with Ad5 knob, suggests that this undesired complex precludes the virus from infecting the cell via CAR-mediation. Alternatively, human-IgG1 incubation was not deleterious to gene transfer of cells by Ad.Zc::Fc-scFv, suggesting that Ad.Cd ability to bind the Fab region of human-IgG1 was providing for targeting ligand concealment or displacement. This experiment prompted further study into the role of Fab and Fc of human IgG molecules in replacing the Fc-scFv fusion protein coupled to the Ad capsid fibers.

Gene Transfer Analysis of Pre-Complexed Ad: :Fc-scFv after Pre-Incubation with Human Fc and Fab.

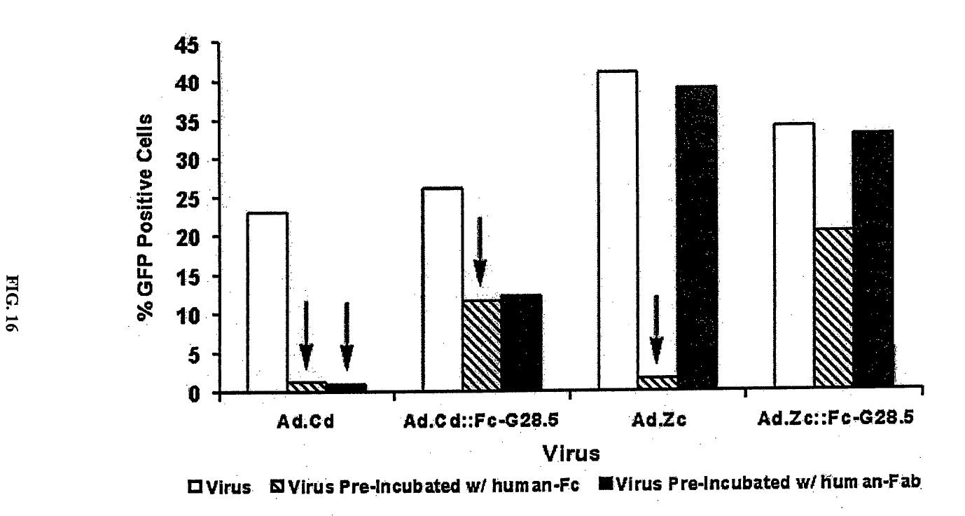

Applicants then sought to determine the role of Fab- and Fc-binding in the displacement of the targeting ligand observed after pre-incubating Ad.Cd::Fc-scFv with human IgG1. To this end, Applicants conducted gene transfer experiments with Ad.Cd and Ad.Zc pre-complexed with Fc-scFv, after incubation with human Fc or human Fab protein. FIG. 16 shows gene transfer data for both viruses, post human-Fc/human-Fab incubation. Surprisingly, for both Ad.Cd::Fc-scFv and Ad.Zc::Fc-scFv, pre-incubation with human-Fc resulted in inhibited gene transfer when compared to the respective control infections. This data suggests that the human-Fc protein competed with and to a significant extent, replaced the Fc-scFv targeting ligand. This result could be attributed to the relatively small size of the human-Fc protein when compared to that of the whole human IgG molecule. Alternatively, after incubation with human-Fab, only Ad.Cd::Fc-scFv displayed decreased efficiency in reporter gene delivery, which again, we attributed to this vector's exclusive ability to bind Fab.

Uncomplexed viruses were also evaluated in this manner, in parallel with their respective controls. As shown in FIG. 16, human-Fc inhibited the gene transfer ability of both Ad.Cd and Ad.Zc, however, only the Cd variant saw a decrease in gene transfer after pre-incubation with Fab. These data also demonstrated that complex formation led to the loss of the vector's ability to recognize the primary Ad5 receptor, CAR. In other words, preformed Ad::Fc-ligand complexes, are not only retargeted via the respective targeting ligand, but are CAR-untargeted as well.

The Fc-binding domain of protein A incorporated at the C-terminus of AdS fiber, provides for a flexible, modular approach to targeting of Ad vectors via Fc-containing ligands (see, e.g., Korokhov et al. (2003) J Virol 77: 12931-40). In this schema, a wide of array of targeted vectors can be developed, without requiring the construction of additional recombinant viruses. Herein, Applicants have further improved upon this documented adenoviral targeting technology (see, e.g., Korokhov et al. (2003) J Virol 77: 12931-40) by modifying the Fc-binding component of this targeting strategy. By abolishing the ability of the vector to bind the Fab regions of immunoglobulin molecules, Applicants have significantly enhanced their two-component targeting system, increasing its attractiveness for scrutiny in in vivo model systems. Fc-binding technology, although novel for adenoviral vectorology (see, e.g., Volpers et al. (2003) J Virol 77: 2093-104 and Korokhov et al. (2003) J Virol 77: 12931-40) has been endeavored in the tropism modification of other viral vectors. This approach was first employed in modifying the coat proteins of retrovirus (see, e.g., Ohno & Meruelo (1997) Biochem Mol Med 62: 123-7) and Sindbis virus (see, e.g., Ohno et al. (1997) Nat Biotechnol 15: 763-7) by inserting, in tandem, two copies of Fc-binding domains from protein A. Coupling of a receptor-specific antibody to these modified vectors resulted in tropism alteration to target cells. Later, adeno-associated virus was similarly engineered to contain a minimized and optimized Fc-binding domain (Z34C), resulting in vector retargeting in vitro via IgG molecules (see, e.g., Ried et al. (2002) J Virol 76: 4559-66). In the development of our pre-complexed targeted Ad vectors, we offer a practical means of purifying Ad-ligand complexes away from free, unbound targeting ligands (see, e.g., Korokhov et al. (2003) J Virol 77: 12931-40). Herein, Applicants have developed highly purified, targeted Ad-ligand complexes, and have displayed their efficiency in CAR-independent gene transfer (see FIGS. 13 and 14). Furthermore, Applicants have also shown that this targeting scheme is unsuccessful in employing murine monoclonal IgG1 molecules for tropism modification. As a result of this finding, Applicants have concluded that the use of human Fc-containing ligands is the most attractive utility of this two-component approach to vector targeting. However, before these vectors could be considered for in vivo application, the C-domain (Cd) of protein A required additional modification to optimize the efficiency in which the vectors maintain their interaction with Fc-containing targeting ligands.

The first problem Applicants attempted to circumvent via modification of the C-domain was the ability of Cd to simultaneously bind multiple Fc or Fab containing ligands. Previous studies have shown that the various domains of protein A, when complexed to the Fc protein, also retain their ability to bind Fab, demonstrating that Fc/Fab interactions with the C-domain are noncompetitive (see, e.g., Starovasnik et al. (1997) Proc Natl Acad Sci U.S.A. 94: 10080-5 and Graille et al. (2000) Proc Natl Acad Sci U.S.A. 97: 5399-404). In the context of our Ad::Fc-ligand pre-formed complexes, the Cd ability to bind Fab would provide for an additional site in which a circulatory IgG molecule could bind to the Ad fiber knob. For an in vivo application, this occurrence would undermine the specificity of the pre-complexed vectors by retargeting them, and thus, render the vector inefficient for cell-specific gene transfer. Therefore, to address this potential hindrance of our system, Applicants attempted to abolish the Fab-binding ability of the knob Cd, while retaining its ability to form highly stable complexes with Fc-containing ligands.

In developing this variation of Ad.Cd (previously known as Ad5.DR-LL-Cd), we considered the findings of affinity studies on the other Fc-binding domains of protein A. The B-domain, exhibiting the same Fc/Fab binding abilities as the C-domain (Cd), has been found to retain Fc affinity while losing Fab affinity, via a single glycine to alanine substitution in its primary amino acid sequence (see, e.g., Jansson et al. (1998) FEMS Immunol Med Microbiol 20: 69-78). Applicants applied this modification to Cd, which Applicants have previously incorporated at the C-terminus of Ad5 fiber. After completing the genetic modification necessary to generate this Z-domain, and its subsequent incorporation at the C-terminus of the knob protein, Applicants developed Ad.Zc with GFP reporter in the E1 region. With this new variant, Applicants have demonstrated the vector's ability to bind the Fc domain, while abolishing its Fab binding characteristics.

Upon constructing the new variant, Applicants sought to address a fundamental criticism of two-component vector targeting, namely, the degree to which these vectors remain bound to their respective targeting ligand in vivo. The studies employing viral vectors other than Ad5, although elegant, offer no suggestion regarding the behavior of these vectors in an in vivo environment (see, e.g., Ohno & Meruelo (1997) Biochem Mol Med 62: 123-7 and Ried et al. (2002) J Virol 76: 4559-66). In this study, Applicants have alluded to the in vivo stability of their adenoviral vectors, by developing in vitro experiments to mimic environments, e.g. the systemic circulation, which are rich in Fc containing immunoglobulins. Applicants have demonstrated that Ad.Zc is superior to their previous Fc-binding vector, retaining its ability to bind the Fc domain with high affinity, and maintaining that interaction when introduced in environment containing a high excess of competing IgG molecules (FIG. 15). To this end, human-IgG1 was employed as the competitive agent, as this isotype quantitatively represents more than half of the IgGs found in the human systemic circulation, and has high affinity for protein A. Applicants' data shows that the pre-formed complex of Fc-scFv and Ad.Zc maintained its transduction efficiency even in the presence of highly concentrated, potential competitor, human-IgG1. Alternatively, the targeting ligand complexed to the previous vector Ad.Cd, when subjected to the same conditions, was unable to recognize the desired marker, CD40.

In view of the inability of Ad.Zc to form stable, pre-formed complexes with murine IgG to achieve tropism alteration, we believe that employing Fc-containing targeting ligands, including humanized antibodies, are an effective means of utilizing this targeting approach. By improving the previous technology in this manner, and eliminating a potentially critical problem of targeting ligand replacement or obstruction via soluble antibodies, Applicants have enhanced the efficacy of two-component targeting in this adenoviral schema. With the findings in this study, Applicants believe that these vectors are attractive candidates for in vivo experimentation, and for further analysis as human gene therapy vectors.

Cell Lines.

293 human embryonal kidney cells and their derivative 293T/17 cells were purchased from the American Type Culture Collection (ATCC, Manassas, Va.). 293 cells expressing human CD40, have been described previously (see, e.g., Belousova et al. (2002) J Virol 76: 8621-31). These cells were cultured and propagated in Dulbecco modified Eagle's Medium-F 12 (DMEM-F12), with 10% FBS, 2 mM glutamine, 100 U of penicillin/ml, and 100 μg of streptomycin/ml. Media and other supplements were purchased from Fisher Scientific (Pittsburgh, Pa.), and FBS was from HyClone (Logan, Utah). All 293 cells and derivatives were cultured at 37° C. in a 5% CO₂ atmosphere.

Antibodies.

4D2 anti-fiber (see, e.g., Hong & Engler (1996) J Virol 70: 7071-8) murine mAb was obtained from Jeffery Engler (University of Alabama at Birmingham). Rabbit anti-Ad2 polyclonal antibodies were purchased from the National Institute of Allergy and Infectious Disease (Bethesda, Md..). Anti-mouse polyclonal antibodies conjugated with horseradish peroxidase were purchased from Amersham Pharmacia Biotech, Inc (Piscataway, N.J.). Penta-His Mab was purchased from Qiagen (Valencia, Calif.). Biotinylated rabbit anti-mouse immunoglobulin polyclonal antibodies and alkaline phosphatase-conjugated streptavidin were both purchased from Jackson ImmunoResearch Laboratories, Inc. (West Grove, Pa.).

Genetic Engineering.

Restriction endonucleases and T4 DNA ligase were purchased from New England Biolabs (Beverly, Mass.). The polymerase chain reaction (PCR) was performed with Pfu DNA polymerase (Stratagene, La Jolla, Calif.). To generate a mutant form of the C-domain with reduced Fab binding, the glycine residue at position 29 was replaced with alanine. To introduce this mutation, two overlapping fragments were generated via PCR using pVS.Fb-Cd DNA as a template and the following pairs of primers: primers Zc-domain-F-ACGTAACGCATTCATCCAAA (SEQ ID NO. 11), pVS.MfeIR-GACTTGAAATTTTCTGCAATTG (SEQ ID NO. 12), and primers BL-F-GGTGGCGGATCCGCGGATAAC (SEQ ID NO. 13), and Zc-domain-R-TTTGGATGAATGCGTTACGT (SEQ ID NO. 14). Zc-domain-F and Zc-domain-R primers are complementary to each other and contain modifications (underlined letters) which, when generated via PCR, result in the substitution of the original GCC triplet (encoding glycine) by a GCA triplet (encoding alanine). The PCR products were purified, mixed and used as templates for amplification with the pVS.MfeIR and BL-F primers. The PCR product representing a sequence encoding part of fibritin molecule fused with the modified C-domain was purified, cleaved with BamHI-MfeI and cloned into BamHI and MfeI digested pKanFb-Cd, resulting in the generation of the shuttle vector pKanFb-Zc. Construction of the pKanFb-Cd and pVS.Fb-Cd plasmids was described previously by Korokhov et al. (see, e.g., Korokhov et al. (2003) J Virol 77: 12931-40).

To express the chimeric proteins in mammalian cells, the BamHI-MfeI fragments from pKanFb-Cd and pKanFb-Zc were transferred into the expression plasmid pVS.FF/CD40L (see, e.g., Belousova et al. (2002) J Virol 76: 8621-31)and were subsequently digested with the same restriction endonucleases. Recombinant Ad genomes incorporating the modified fiber genes were derived by homologous DNA recombination in Escherichia coli BJ5183 with SwaI-linearized plasmid pVL4000, as described previously (see, e.g., Chartier et al. (1996) J Virol 70: 4805-10). pVL4000 is a derivative of pTG3602 (see, e.g., Chartier et al. (1996) J Virol 70: 4805-10), which contains an Ad5 genome with E1 and the fiber gene deleted. In place of the deleted E1 region, the genome contains a CMV immediate-early promoter driving the green fluorescent protein (GFP) gene.

Viruses.

As previously described, Ad vectors were generated by transfecting 293 cells with PacI-digested Ad rescue vectors (see, e.g., Krasnykh et al. (1998) J Virol 72: 1844-52). The vectors were purified by ultra-centrifugation in CsCl gradients, according to a previously described protocol (see, e.g., Graham & Prevec (1995) Mol Biotechnol 3: 207-20). To determine the concentrations of viral preparations, the Lowry-based DC protein assay (Bio-Rad, Hercules, Calif.) was used, with purified BSA as a standard.

Recombinant Proteins.

The design, expression, and purification of the Fc-G28.5 protein, consisting of an anti-human CD40 single chain antibody (scFv) G28.5 (see, e.g., Pereboev et al. (2002) Gene Ther 9: 1189-93) fused with the Fc domain of human immunoglobulin, have previously been reported (see, e.g., Korokhov et al. (2003) J Virol 77: 12931-40). The final protein concentration was determined using the DC protein assay (Bio-Rad) with standard BSA.

Preparation of Pre-Formed Viral Complexes.

Ad vectors complexed with Fc-containing targeting ligands were generated according to previously described methods (see, e.g., Korokhov et al. (2003) J Virol 77: 12931-40). Briefly, after the first CsCl ultracentrifugation (3 hr at 4° and 25,000 rpm) of cell lysates infected with Ad.Cd and Ad.Zc, the collected viruses were incubated in vitro at room temperature with Fc-G28.5 or anti-CD40 mouse monoclonal antibody (G28.5), at a concentration equaling 50× the number of targeting ligands per capsid vertex. After 30 minutes of incubation with the appropriate targeting ligand, the samples were loaded onto a second CsCl gradient and were spun overnight at the same conditions. Concentrations of the viral preparations were determined using the DC protein assay, and were then stored at −80° C. until used.

Transient Expression of Modified Fiber Proteins.

293T/17 cells were transfected with the pVS-derived expression vectors using the DOTAP liposomal transfection reagent (Roche, Mannheim, Germany) according to the manufacturer's protocol. At 72 hours post-transfection the cells were washed with PBS, harvested, and lysed in cell culture lysis reagent (Promega, Madison, Wis.) at 10⁶ cells/ml. Cell lysates were used in enzyme-linked immunosorbent assays (ELISAs) and for Western blotting.

Western Blot.

Samples were incubated in Laemmli sample buffer at 96° C. for 5 min and separated on 4-20% gradient polyacrylamide gel (Bio-Rad). The proteins were electroblotted onto polyvinylidene difluoride (PVDF) membrane and the blots were developed with the WesternBreeze immunodetection system (Invitrogen) according to the manufacturer's protocol using either the 4D2, Penta-His, or anti-murine IgG antibodies as primary probes.

ELISA.

The wells of 96-well Nunc Immuno-plates (Fisher Scientific) were coated overnight at 4° C. with proteins diluted in 50 mM carbonate buffer (pH 8.6) at a concentration of 5 μg/ml. The unsaturated surface of the wells was then blocked for 1 h at RT by the addition of 200 μl of blocking buffer (Tris-buffered saline, TBS, with 0.05% Tween 20 and 0.5% casein) to each well. The blocking buffer was replaced with 100 μl of cell lysates or Ad preparations diluted in binding buffer (TBS with 0.05% Tween 20 and 0.05% casein). Plates were incubated at RT for 1 h and then were washed four times with washing buffer (TBS with 0.05% Tween 20). Bound fiber proteins or Ad particles were detected by incubation for 1 h at RT with 4D2 mAb or anti-Ad2 polyclonal antibodies, respectively. The wells were washed four times with washing buffer and then either the goat anti-mouse immunoglobulin G or goat anti-rabbit immunoglobulin antibodies conjugated with horseradish peroxidase (HRP) (Dako Corporation, Carpinteria, CA) were added and incubation was continued for 1 h. The color was developed with the Sigma FAST o-phenylenediamine dihydrochloride tablet kit (Sigma, St Louis, Mo.) as recommended by the manufacturer. The color intensity was measured at 490 nm with an EL800 plate reader (Bio-Tek Instruments, Winooski, Vt.).

Gene Transfer Assay.

To examine the gene delivery of green fluorescent protein (GFP) via Ad, 5×10⁵ cells (293-CD40) were grown in 24-well, poly-lysine plates at 37° C. For CAR blocking assays employing Ad5 knob protein, wells were incubated with 200:1 of 2% FBS-DMEM at a concentration of 100 μg/ml recombinant protein for 10 minutes at room temperature. Cells were infected at an MOI of 40 or 100 vp/cell diluted in 2% FBS-DMEM for 30 minutes at room temperature. The infection media were then aspirated, and wells were washed with 0.5ml of 2% FBS-DMEM once. 1 ml of medium was added to the wells and the cells were incubated at 37° C. for 48 hours to allow for GFP expression. Post-incubation, the cells were prepared for FACS analysis.

Preparation/Analysis of FACS Samples.

To remove cells from plates, the wells were incubated with 0.5ml of CellStripper (Mediatech, Herdon, Va.) for 10 minutes until all cells were detached from the well surface. To each well, 1.0 ml of 2% FBS-DMEM was added and the cell suspension was transferred to a culture tube, and spun at 5000 rpm for 5 minutes at 4° C. The media were then aspirated from the pelleted cells, which where then resuspended in 4 ml of FACS buffer (0.1% BSA, 0.01% NaN3 in PBS). The cells were spun at the same conditions, the buffer was aspirated, and the samples were resuspended in 300 μl of FACS buffer. To determine GFP expression, samples were then analyzed by flow cytometry in the University of Alabama at Birmingham FACS Core Facility on a FACSCalibur machine using Cell quest FACS analysis software (Becton-Dickinson, Franklin Lakes, N.J., U.S.A.). GFP was measured over the FITC detection channel at a wavelength of 530 nm. GFP expression reflected in the results section represents the percent GFP detected in gated, live cells.

### Example 15

### Sequence of Ad-Zc

| | | |
|---|---|---|
| 1 | taannntccc ttccagctct ctgccccttt tggattgaag ccaatatgat aatgaggggg | (SEQ ID NO:15) |
| 61 | tggagtttgt gacgtggcgc gggcgtggga acggggcggg tgacgtagta gtgtggcgga | |
| 121 | agtgtgatgt tgcaagtgtg gcggaacaca tgtaagcgac ggatgtggca aaagtgacgt | |
| 181 | ttttggtgtg cgccggtgta cacaggaagt gacaattttc gcgcggtttt aggcggatgt | |
| 241 | tgtagtaaat ttgggcgtaa ccgagtaaga tttggccatt ttcgcgggaa aactgaataa | |
| 301 | gaggaagtga aatctgaata attttgtgtt actcatagcg cgtaannncg cgttaagata | |
| 361 | cattgatgag tttggacaaa ccacaactag aatgcagtga aaaaaatgct ttatttgtga | |
| 421 | aatttgtgat gctattgctt tatttgtaac cattataagc tgcaataaac aagttaacaa | |
| 481 | caacaattgc attcatttta tgtttcaggt tcagggggag gtgtgggagg ttttttaaag | |
| 541 | caagtaaaac ctctacaaat gtggtatggc tgattatgat cagttatcta gatccggtgg | |
| 601 | atctgagtcc ggacttgtac agctcgtcca tgccgagagt gatcccggcg gcggtcacga | |
| 661 | actccagcag gaccatgtga tcgcgcttct cgttggggtc tttgctcagg gcggactggg | |
| 721 | tgctcaggta gtggttgtcg ggcagcagca cggggccgtc gccgatgggg gtgttctgct | |
| 781 | ggtagtggtc ggcgagctgc acgctgccgt cctcgatgtt gtggcggatc ttgaagttca | |
| 841 | ccttgatgcc gttcttctgc ttgtcggcca tgatatagac gttgtggctg ttgtagttgt | |
| 901 | actccagctt gtgccccagg atgttgccgt cctccttgaa gtcgatgccc ttcagctcga | |
| 961 | tgcggttcac cagggtgtcg ccctcgaact tcacctcggc gcgggtcttg tagttgccgt | |
| 1021 | cgtccttgaa gaagatggtg cgctcctgga cgtagccttc gggcatggcg gacttgaaga | |
| 1081 | agtcgtgctg cttcatgtgg tcggggtagc ggctgaagca ctgcacgccg taggtcaggg | |
| 1141 | tggtcacgag ggtgggccag ggcacgggca gcttgccggt ggtgcagatg aacttcaggg | |
| 1201 | tcagcttgcc gtaggtggca tcgccctcgc cctcgccgga cacgctgaac ttgtggccgt | |
| 1261 | ttacgtcgcc gtccagctcg accaggatgg gcaccacccc ggtgaacagc tcctcgccct | |
| 1321 | tgctcaccat ggtggcgacc ggtagcgcta gcggatctga cggttcacta aaccagctct | |
| 1381 | gcttatatag acctcccacc gtacacgcct accgcccatt tgcgtcaatg gggcggagtt | |
| 1441 | gttacgacat tttggaaagt cccgttgatt ttggtgccaa aacaaactcc cattgacgtc | |
| 1501 | aatggggtgg agacttggaa atccccgtga gtcaaaccgc tatccacgcc cattgatgta | |
| 1561 | ctgccaaaac cgcatcacca tggtaatagc gatgactaat acgtagatgt actgccaagt | |
| 1621 | aggaaagtcc cataaggtca tgtactgggc ataatgccag gcgggccatt taccgtcatt | |
| 1681 | gacgtcaata gggggcgtac ttggcatatg atacacttga tgtactgcca agtgggcagt | |
| 1741 | ttaccgtaaa tactccaccc attgacgtca atggaaagtc cctattggcg ttactatggg | |
| 1801 | aacatacgtc attattgacg tcaatgggcg ggggtcgttg ggcggtcagc caggcgggcc | |
| 1861 | atttaccaac gcggaactcc atatatgggc tatgaactaa tgaccccgta attgattact | |
| 1921 | attannntaa gggtgggaaa gaatatataa ggtgggggtc ttatgtagtt ttgtatctgt | |
| 1981 | tttgcagcag ccgccgccgc catgagcacc aactcgtttg atggaagcat tgtgagctca | |
| 2041 | tatttgacaa cgcgcatgcc cccatgggcc ggggtgcgtc agaatgtgat gggctccagc | |
| 2101 | attgatggtc gccccgtcct gcccgcaaac tctactacct tgacctacga gaccgtgtct | |
| 2161 | ggaacgccgt tggagactgc agcctccgcc gccgcttcag ccgctgcagc caccgcccgc | |
| 2221 | gggattgtga ctgactttgc tttcctgagc ccgcttgcaa gcagtgcagc ttcccgttca | |
| 2281 | tccgcccgcg atgacaagtt gacggctctt ttggcacaat tggattcttt gacccgggaa | |
| 2341 | cttaatgtcg tttctcagca gctgttggat ctgcgccagc aggtttctgc cctgaaggct | |
| 2401 | tcctcccctc ccaatgcggt ttaaaacata aataaaaaac cagactctgt ttggatttgg | |
| 2461 | atcaagcaag tgtcttgctg tctttattta ggggttttgc gcgcgcggta ggcccgggac | |
| 2521 | cagcggtctc ggtcgttgag ggtcctgtgt attttttcca ggacgtggta aaggtgactc | |
| 2581 | tggatgttca gatacatggg cataagcccg tctctggggt ggaggtagca ccactgcaga | |
| 2641 | gcttcatgct gcggggtggt gttgtagatg atccagtcgt agcaggagcg ctgggcgtgg | |
| 2701 | tgcctaaaaa tgtctttcag tagcaagctg attgccaggg gcaggccctt ggtgtaagtg | |
| 2761 | tttacaaagc ggttaagctg ggatgggtgc atacgtgggg atatgagatg catcttggac | |
| 2821 | tgtattttta ggttggctat gttcccagcc atatccctcc ggggattcat gttgtgcaga | |
| 2881 | accaccagca cagtgtatcc ggtgcacttg ggaaatttgt catgtagctt agaaggaaat | |
| 2941 | gcgtggaaga acttggagac gcccttgtga cctccaagat tttccatgca ttcgtccata | |
| 3001 | atgatggcaa tgggcccacg ggcggcggcc tgggcgaaga tatttctggg atcactaacg | |
| 3061 | tcatagttgt gttccaggat gagatcgtca taggccattt ttacaaagcg cgggcggagg | |
| 3121 | gtgccagact gcggtataat ggttccatcc ggcccagggg cgtagttacc ctcacagatt | |
| 3181 | tgcatttccc acgctttgag ttcagatggg gggatcatgt ctacctgcgg ggcgatgaag | |
| 3241 | aaaacggttt ccggggtagg ggagatcagc tgggaagaaa gcaggttcct gagcagctgc | |
| 3301 | gacttaccgc agccggtggg cccgtaaatc acacctatta ccgggtgcaa ctggtagtta | |
| 3361 | agagagctgc agctgccgtc atccctgagc aggggggcca cttcgttaag catgtccctg | |
| 3421 | actcgcatgt tttccctgac caaatccgcc agaaggcgct cgccgcccag cgatagcagt | |
| 3481 | tcttgcaagg aagcaaagtt tttcaacggt ttgagaccgt ccgccgtagg catgcttttg | |
| 3541 | agcgtttgac caagcagttc caggcggtcc cacagctcgg tcacctgctc tacggcatct | |
| 3601 | cgatccagca tatctcctcg tttcgcgggt tggggcggct ttcgctgtac ggcagtagtc | |
| 3661 | ggtgctcgtc cagacgggcc agggtcatgt ctttccacgg gcgcagggtc ctcgtcagcg | |
| 3721 | tagtctgggt cacggtgaag gggtgcgctc cgggctgcgc gctggccagg gtgcgcttga | |
| 3781 | ggctggtcct gctggtgctg aagcgctgcc ggtcttcgcc ctgcgcgtcg gccaggtagc | |
| 3841 | atttgaccat ggtgtcatag tccagcccct ccgcggcgtg gcccttggcg cgcagcttgc | |
| 3901 | ccttggagga ggcgccgcac gaggggcagt gcagactttt gagggcgtag agcttgggcg | |
| 3961 | cgagaaatac cgattccggg gagtaggcat ccgcgccgca ggccccgcag acggtctcgc | |
| 4021 | attccacgag ccaggtgagc tctggccgtt cggggtcaaa aaccaggttt cccccatgct | |
| 4081 | ttttgatgcg tttcttacct ctggtttcca tgagccggtg tccacgctcg gtgacgaaaa | |
| 4141 | ggctgtccgt gtccccgtat acagacttga gaggcctgtc ctcgagcggt gttccgcggt | |
| 4201 | cctcctcgta tagaaactcg gaccactctg agacaaaggc tcgcgtccag gccagcacga | |
| 4261 | aggaggctaa gtgggagggg tagcggtcgt tgtccactag ggggtccact cgctccaggg | |
| 4321 | tgtgaagaca catgtcgccc tcttcggcat caaggaaggt gattggtttg taggtgtagg | |
| 4381 | ccacgtgacc gggtgttcct gaaggggggc tataaaaggg ggtgggggcg cgttcgtcct | |
| 4441 | cactctcttc cgcatcgctg tctgcgaggg ccagctgttg gggtgagtac tccctctgaa | |
| 4501 | aagcgggcat gacttctgcc taagattgtc agtttccaaa aacgaggagg atttgatatt | |
| 4561 | cacctggccc gcggtgatgc ctttgagggt ggccgcatcc atctggtcag aaaagacaat | |
| 4621 | ctttttgttg tcaagcttgg tggcaaacga cccgtagagg gcgttggaca gcaacttggc | |
| 4681 | gatggagcgc agggtttggt ttttgtcgcg atcggcgcgc tccttggccg cgatgtttag | |
| 4741 | ctgcacgtat tcgcgcgcaa cgcaccgcca ttcgggaaag acggtggtgc gctcgtcggg | |
| 4801 | caccaggtgc acgcgccaac cgcggttgtg cagggtgaca aggtcaacgc tggtggctac | |
| 4861 | ctctccgcgt aggcgctcgt tggtccagca gaggcggccg cccttgcgcg agcagaatgg | |
| 4921 | cggtaggggg tctagctgcg tctcgtccgg ggggtctgcg tccacggtaa agaccccggg | |
| 4981 | cagcaggcgc gcgtcgaagt agtctatctt gcatccttgc aagtctagcg cctgctgcca | |
| 5041 | tgcgcgggcg gcaagcgcgc gctcgtatgg gttgagtggg ggaccccatg gcatggggtg | |
| 5101 | ggtgagcgcg gaggcgtaca tgccgcaaat gtcgtaaacg tagaggggct ctctgagtat | |
| 5161 | tccaagatat gtagggtagc atcttccacc gcggatgctg gcgcgcacgt aatcgtatag | |
| 5221 | ttcgtgcgag ggagcgagga ggtcgggacc gaggttgcta cgggcgggct gctctgctcg | |
| 5281 | gaagactatc tgcctgaaga tggcatgtga gttggatgat atggttggac gctggaagac | |
| 5341 | gttgaagctg gcgtctgtga gacctaccgc gtcacgcacg aaggaggcgt aggagtcgcg | |
| 5401 | cagcttgttg accagctcgg cggtgacctg cacgtctagg gcgcagtagt ccagggtttc | |
| 5461 | cttgatgatg tcatacttat cctgtccctt ttttttccac agctcgcggt tgaggacaaa | |
| 5521 | ctcttcgcgg tctttccagt actcttggat cggaaacccg tcggcctccg aacggtaaga | |
| 5581 | gcctagcatg tagaactggt tgacggcctg gtaggcgcag catccctttt ctacgggtag | |
| 5641 | cgcgtatgcc tgcgcggcct tccggagcga ggtgtgggtg agcgcaaagg tgtccctgac | |
| 5701 | catgactttg aggtactggt atttgaagtc agtgtcgtcg catccgccct gctcccagag | |
| 5761 | caaaaagtcc gtgcgctttt tggaacgcgg atttggcagg gcgaaggtga catcgttgaa | |
| 5821 | gagtatcttt cccgcgcgag gcataaagtt gcgtgtgatg cggaagggtc ccggcacctc | |
| 5881 | ggaacggttg ttaattacct gggcggcgag cacgatctcg tcaaagccgt tgatgttgtg | |
| 5941 | gcccacaatg taaagttcca agaagcgcgg gatgcccttg atggaaggca attttttaag | |
| 6001 | ttcctcgtag gtgagctctt caggggagct gagcccgtgc tctgaaaggg cccagtctgc | |
| 6061 | aagatgaggg ttggaagcga cgaatgagct ccacaggtca cgggccatta gcatttgcag | |
| 6121 | gtggtcgcga aaggtcctaa actggcgacc tatggccatt ttttctgggg tgatgcagta | |
| 6181 | gaaggtaagc gggtcttgtt cccagcggtc ccatccaagg ttcgcggcta ggtctcgcgc | |
| 6241 | ggcagtcact agaggctcat ctccgccgaa cttcatgacc agcatgaagg gcacgagctg | |
| 6301 | cttcccaaag gcccccatcc aagtataggt ctctacatcg taggtgacaa agagacgctc | |
| 6361 | ggtgcgagga tgcgagccga tcgggaagaa ctggatctcc cgccaccaat tggaggagtg | |
| 6421 | gctattgatg tggtgaaagt agaagtccct gcgacgggcc gaacactcgt gctggctttt | |
| 6481 | gtaaaaacgt gcgcagtact ggcagcggtg cacgggctgt acatcctgca cgaggttgac | |
| 6541 | ctgacgaccg cgcacaagga agcagagggg aatttgagcc cctcgcctgg cgggtttggc | |
| 6601 | tggtggtctt ctacttcggc tgcttgtcct tgaccgtctg gctgctcgag gggagttacg | |
| 6661 | gtggatcgga ccaccacgcc gcgcgagccc aaagtccaga tgtccgcgcg cggcggtcgg | |
| 6721 | agcttgatga caacatcgcg cagatgggag ctgtccatgg tctggagctc ccgcggcgtc | |
| 6781 | aggtcaggcg ggagctcctg caggtttacc tcgcatagac gggtcagggc gcgggctaga | |
| 6841 | tccaggtgat acctaatttc caggggctgg ttggtggcgg cgtcgatggc ttgcaagagg | |
| 6901 | ccgcatcccc gcggcgcgac tacggtaccg cgcggcgggc ggtgggccgc gggggtgtcc | |
| 6961 | ttggatgatg catctaaaag cggtgacgcg ggcgagcccc cggaggtagg gggggctccg | |
| 7021 | gacccgccgg gagagggggc aggggcacgt cggcgccgcg cgcgggcagg agctggtgct | |
| 7081 | gcgcgcgtag gttgctggcg aacgcgacga cgcggcggtt gatctcctga atctggcgcc | |
| 7141 | tctgcgtgaa gacgacgggc ccggtgagct tgagcctgaa agagagttcg acagaatcaa | |
| 7201 | tttcggtgtc gttgacggcg gcctggcgca aaatctcctg cacgtctcct gagttgtctt | |
| 7261 | gataggcgat ctcggccatg aactgctcga tctcttcctc ctggagatct ccgcgtccgg | |
| 7321 | ctcgctccac ggtggcggcg aggtcgttgg aaatgcgggc catgagctgc gagaaggcgt | |
| 7381 | tgaggcctcc ctcgttccag acgcggctgt agaccacgcc cccttcggca tcgcgggcgc | |
| 7441 | gcatgaccac ctgcgcgaga ttgagctcca cgtgccgggc gaagacggcg tagtttcgca | |
| 7501 | ggcgctgaaa gaggtagttg agggtggtgg cggtgtgttc tgccacgaag aagtacataa | |
| 7561 | cccagcgtcg caacgtggat tcgttgatat cccccaaggc ctcaaggcgc tccatggcct | |
| 7621 | cgtagaagtc cacggcgaag ttgaaaaact gggagttgcg cgccgacacg gttaactcct | |
| 7681 | cctccagaag acggatgagc tcggcgacag tgtcgcgcac ctcgcgctca aaggctacag | |
| 7741 | gggcctcttc ttcttcttca atctcctctt ccataagggc ctccccttct tcttcttctg | |
| 7801 | gcggcggtgg gggagggggg acacggcggc gacgacggcg caccgggagg cggtcgacaa | |
| 7861 | agcgctcgat catctccccg cggcgacggc gcatggtctc ggtgacggcg cggccgttct | |
| 7921 | cgcgggggcg cagttggaag acgccgcccg tcatgtcccg gttatgggtt ggcggggggc | |
| 7981 | tgccatgcgg cagggatacg gcgctaacga tgcatctcaa caattgttgt gtaggtactc | |
| 8041 | cgccgccgag ggacctgagc gagtccgcat cgaccggatc ggaaaacctc tcgagaaagg | |
| 8101 | cgtctaacca gtcacagtcg caaggtaggc tgagcaccgt ggcgggcggc agcgggcggc | |
| 8161 | ggtcggggtt gtttctggcg gaggtgctgc tgatgatgta attaaagtag gcggtcttga | |
| 8221 | gacggcggat ggtcgacaga agcaccatgt ccttgggtcc ggcctgctga atgcgcaggc | |
| 8281 | ggtcggccat gccccaggct tcgttttgac atcggcgcag gtctttgtag tagtcttgca | |
| 8341 | tgagcctttc taccggcact tcttcttctc cttcctcttg tcctgcatct cttgcatcta | |
| 8401 | tcgctgcggc ggcggcggag tttggccgta ggtggcgccc tcttcctccc atgcgtgtga | |
| 8461 | ccccgaagcc cctcatcggc tgaagcaggg ctaggtcggc gacaacgcgc tcggctaata | |
| 8521 | tggcctgctg cacctgcgtg agggtagact ggaagtcatc catgtccaca aagcggtggt | |
| 8581 | atgcgcccgt gttgatggtg taagtgcagt tggcctaacg gaccagttaa cggtctggtg | |
| 8641 | acccggctgc gagagctcgg tgtacctgag acgcgagtaa gccctcgagt caaatacgta | |
| 8701 | gtcgttgcaa gtccgcacca ggtactggta tcccaccaaa aagtgcggcg gcggctggcg | |
| 8761 | gtagaggggc cagcgtaggg tggccggggc tccgggggcg agatcttcca acataaggcg | |
| 8821 | atgatatccg tagatgtacc tggacatcca ggtgatgccg gcggcggtgg tggaggcgcg | |
| 8881 | cggaaagtcg cggacgcggt tccagatgtt gcgcagcggc aaaaagtgct ccatggtcgg | |
| 8941 | gacgctctgg ccggtcaggc gcgcgcaatc gttgacgctc tagaccgtgc aaaaggagag | |
| 9001 | cctgtaagcg ggcactcttc cgtggtctgg tggataaatt cgcaagggta tcatggcgga | |
| 9061 | cgaccggggt tcgagccccg tatccggccg tccgccgtga tccatgcggt taccgcccgc | |
| 9121 | gtgtcgaacc caggtgtgcg acgtcagaca acgggggagt gctccttttg gcttccttcc | |
| 9181 | aggcgcggcg gctgctgcgc tagctttttt ggccactggc cgcgcgcagc gtaagcggtt | |
| 9241 | aggctggaaa gcgaaagcat taagtggctc gctccctgta gccggagggt tattttccaa | |
| 9301 | gggttgagtc gcgggacccc cggttcgagt ctcggaccgg ccggactgcg gcgaacgggg | |
| 9361 | gtttgcctcc ccgtcatgca agaccccgct tgcaaattcc tccggaaaca gggacgagcc | |
| 9421 | ccttttttgc ttttcccaga tgcatccggt gctgcggcag atgcgccccc ctcctcagca | |
| 9481 | gcggcaagag caagagcagc ggcagacatg cagggcaccc tcccctcctc ctaccgcgtc | |
| 9541 | aggaggggcg acatccgcgg ttgacgcggc agcagatggt gattacgaac ccccgcggcg | |
| 9601 | ccgggcccgg cactacctgg acttggagga gggcgagggc ctggcgcggc taggagcgcc | |
| 9661 | ctctcctgag cggtacccaa gggtgcagct gaagcgtgat acgcgtgagg cgtacgtgcc | |
| 9721 | gcggcagaac ctgtttcgcg accgcgaggg agaggagccc gaggagatgc gggatcgaaa | |
| 9781 | gttccacgca gggcgcgagc tgcggcatgg cctgaatcgc gagcggttgc tgcgcgagga | |
| 9841 | ggactttgag cccgacgcgc gaaccgggat tagtcccgcg cgcgcacacg tggcggccgc | |
| 9901 | cgacctggta accgcatacg agcagacggt gaaccaggag attaactttc aaaaaagctt | |
| 9961 | taacaaccac gtgcgtacgc ttgtggcgcg cgaggaggtg gctataggac tgatgcatct | |
| 10021 | gtgggacttt gtaagcgcgc tggagcaaaa cccaaatagc aagccgctca tggcgcagct | |
| 10081 | gttccttata gtgcagcaca gcagggacaa cgaggcattc agggatgcgc tgctaaacat | |
| 10141 | agtagagccc gagggccgct ggctgctcga tttgataaac atcctgcaga gcatagtggt | |
| 10201 | gcaggagcgc agcttgagcc tggctgacaa ggtggccgcc atcaactatt ccatgcttag | |
| 10261 | cctgggcaag ttttacgccc gcaagatata ccatacccct tacgttccca tagacaagga | |
| 10321 | ggtaaagatc gaggggttct acatgcgcat ggcgctgaag gtgcttacct tgagcgacga | |
| 10381 | cctgggcgtt tatcgcaacg agcgcatcca caaggccgtg agcgtgagcc ggcggcgcga | |
| 10441 | gctcagcgac cgcgagctga tgcacagcct gcaaagggcc ctggctggca cgggcagcgg | |
| 10501 | cgatagagag gccgagtcct actttgacgc gggcgctgac ctgcgctggg ccccaagccg | |
| 10561 | acgcgccctg gaggcagctg gggccggacc tgggctggcg gtggcacccg cgcgcgctgg | |
| 10621 | caacgtcggc ggcgtggagg aatatgacga ggacgatgag tacagccaga ggacggcgag | |
| 10681 | tactaagcgg tgatgtttct gatcagatga tgcaagacgc aacggacccg gcggtgcggg | |
| 10741 | cggcgctgca gagccagccg tccggcctta actccacgga cgactggcgc caggtcatgg | |
| 10801 | accgcatcat gtcgctgact gcgcgcaatc ctgacgcgtt ccggcagcag ccgcaggcca | |
| 10861 | accggctctc cgcaattctg gaagcggtgg tcccggcgcg cgcaaacccc acgcacgaga | |
| 10921 | aggtgctggc gatcgtaaac gcgctggccg aaaacagggc catccggccc gacgaggccg | |
| 10981 | gcctggtcta cgacgcgctg cttcagcgcg tggctcgtta caacagcggc aacgtgcaga | |
| 11041 | ccaacctgga ccggctggtg ggggatgtgc gcgaggccgt ggcgcagcgt gagcgcgcgc | |
| 11101 | agcagcaggg caacctgggc tccatggttg cactaaacgc cttcctgagt acacagcccg | |
| 11161 | ccaacgtgcc gcggggacag gaggactaca ccaactttgt gagcgcactg cggctaatgg | |
| 11221 | tgactgagac accgcaaagt gaggtgtacc agtctgggcc agactatttt ttccagacca | |
| 11281 | gtagacaagg cctgcagacc gtaaacctga gccaggcttt caaaaacttg caggggctgt | |
| 11341 | ggggggtgcg ggctcccaca ggcgaccgcg cgaccgtgtc tagcttgctg acgcccaact | |
| 11401 | cgcgcctgtt gctgctgcta atagcgccct tcacggacag tggcagcgtg tcccgggaca | |
| 11461 | catacctagg tcacttgctg acactgtacc gcgaggccat aggtcaggcg catgtggacg | |
| 11521 | agcatacttt ccaggagatt acaagtgtca gccgcgcgct ggggcaggag gacacgggca | |
| 11581 | gcctggaggc aaccctaaac tacctgctga ccaaccggcg gcagaagatc ccctcgttgc | |
| 11641 | acagtttaaa cagcgaggag gagcgcattt tgcgctacgt gcagcagagc gtgagcctta | |
| 11701 | acctgatgcg cgacggggta acgcccagcg tggcgctgga catgaccgcg cgcaacatgg | |
| 11761 | aaccgggcat gtatgcctca aaccggccgt ttatcaaccg cctaatggac tacttgcatc | |
| 11821 | gcgcggccgc cgtgaacccc gagtatttca ccaatgccat cttgaacccg cactggctac | |
| 11881 | cgccccctgg tttctacacc gggggattcg aggtgcccga gggtaacgat ggattcctct | |
| 11941 | gggacgacat agacgacagc gtgttttccc cgcaaccgca gaccctgcta gagttgcaac | |
| 12001 | agcgcgagca ggcagaggcg gcgctgcgaa aggaaagctt ccgcaggcca agcagcttgt | |
| 12061 | ccgatctagg cgctgcggcc ccgcggtcag atgctagtag cccatttcca agcttgatag | |
| 12121 | ggtctcttac cagcactcgc accacccgcc cgcgcctgct gggcgaggag gagtacctaa | |
| 12181 | acaactcgct gctgcagccg cagcgcgaaa aaaacctgcc tccggcattt cccaacaacg | |
| 12241 | ggatagagag cctagtggac aagatgagta gatggaagac gtacgcgcag gagcacaggg | |
| 12301 | acgtgccagg cccgcgcccg cccacccgtc gtcaaaggca cgaccgtcag cggggtctgg | |
| 12361 | tgtgggagga cgatgactcg gcagacgaca gcagcgtcct ggatttggga gggagtggca | |
| 12421 | acccgtttgc gcaccttcgc cccaggctgg ggagaatgtt ttaaaaaaaa aaaagcatga | |
| 12481 | tgcaaaataa aaaactcacc aaggccatgg caccgagcgt tggttttctt gtattcccct | |
| 12541 | tagtatgcgg cgcgcggcga tgtatgagga aggtcctcct ccctcctacg agagtgtggt | |
| 12601 | gagcgcggcg ccagtggcgg cggcgctggg ttctcccttc gatgctcccc tggacccgcc | |
| 12661 | gtttgtgcct ccgcggtacc tgcggcctac cggggggaga aacagcatcc gtactctgag | |
| 12721 | ttggcacccc tattcgacac cacccgtgtg tacctggtgg acaacaagtc aacggatgtg | |
| 12781 | gcatccctga actaccagaa cgaccacagc aactttctga ccacggtcat tcaaaacaat | |
| 12841 | gactacagcc cgggggaggc aagcacacag accatcaatc ttgacgaccg gtcgcactgg | |
| 12901 | ggcggcgacc tgaaaaccat cctgcatacc aacatgccaa atgtgaacga gttcatgttt | |
| 12961 | accaataagt ttaaggcgcg ggtgatggtg tcgcgcttgc ctactaagga caatcaggtg | |
| 13021 | gagctgaaat acgagtgggt ggagttcacg ctgcccgagg gcaactactc cgagaccatg | |
| 13081 | accatagacc ttatgaacaa cgcgatcgtg gagcactact tgaaagtggg cagacagaac | |
| 13141 | ggggttctgg aaagcgacat cggggtaaag tttgacaccc gcaacttcag actggggttt | |
| 13201 | gaccccgtca ctggtcttgt catgcctggg gtatatacaa acgaagcctt ccatccagac | |
| 13261 | atcattttgc tgccaggatg cggggtggac ttcacccaca gccgcctgag caacttgttg | |
| 13321 | ggcatccgca agcggcaacc cttccaggag ggctttagga tcacctacga tgatctggag | |
| 13381 | ggtggtaaca ttcccgcact gttggatgtg gacgcctacc aggcgagctt gaaagatgac | |
| 13441 | accgaacagg gcgggggtgg cgcaggcggc agcaacagca gtggcagcgg cgcggaagag | |
| 13501 | aactccaacg cggcagccgc ggcaatgcag ccggtggagg acatgaacga tcatgccatt | |
| 13561 | cgcggcgaca cctttgccac acgggctgag gagaagcgcg ctgaggccga agcagcggcc | |
| 13621 | gaagctgccg cccccgctgc gcaacccgag gtcgagaagc ctcagaagaa accggtgatc | |
| 13681 | aaacccctga cagaggacag caagaaacgc agttacaacc taataagcaa tgacagcacc | |
| 13741 | ttcacccagt accgcagctg gtaccttgca tacaactacg gcgaccctca gaccggaatc | |
| 13801 | cgctcatgga ccctgctttg cactcctgac gtaacctgcg gctcggagca ggtctactgg | |
| 13861 | tcgttgccag acatgatgca agaccccgtg accttccgct ccacgcgcca gatcagcaac | |
| 13921 | tttccggtgg tgggcgccga gctgttgccc gtgcactcca agagcttcta caacgaccag | |
| 13981 | gccgtctact cccaactcat ccgccagttt acctctctga cccacgtgtt caatcgcttt | |
| 14041 | cccgagaacc agattttggc gcgcccgcca gcccccacca tcaccaccgt cagtgaaaac | |
| 14101 | gttcctgctc tcacagatca cgggacgcta ccgctgcgca acagcatcgg aggagtccag | |
| 14161 | cgagtgacca ttactgacgc cagacgccgc acctgcccct acgtttacaa ggccctgggc | |
| 14221 | atagtctcgc cgcgcgtcct atcgagccgc actttttgag caagcatgtc catccttata | |
| 14281 | tcgcccagca ataacacagg ctggggcctg cgcttcccaa gcaagatgtt tggcggggcc | |
| 14341 | aagaagcgct ccgaccaaca cccagtgcgc gtgcgcgggc actaccgcgc gccctggggc | |
| 14401 | gcgcacaaac gcggccgcac tgggcgcacc accgtcgatg acgccatcga cgcggtggtg | |
| 14461 | gaggaggcgc gcaactacac gcccacgccg ccaccagtgt ccacagtgga cgcggccatt | |
| 14521 | cagaccgtgg tgcgcggagc ccggcgctat gctaaaatga agagacggcg gaggcgcgta | |
| 14581 | gcacgtcgcc accgccgccg acccggcact gccgcccaac gcgcggcggc ggccctgctt | |
| 14641 | aaccgcgcac gtcgcaccgg ccgacgggcg gccatgcggg ccgctcgaag gctggccgcg | |
| 14701 | ggtattgtca ctgtgccccc caggtccagg cgacgagcgg ccgccgcagc agccgcggca | |
| 14761 | ttagtgctat gactcagggt cgcaggggca acgtgtattg ggtgcgcgac tcggttagcg | |
| 14821 | gcctgcgcgt gcccgtgcgc acccgccccc cgcgcaacta gattgcaaga aaaaactact | |
| 14881 | tagactcgta ctgttgtatg tatccagcgg cggcggcgcg caacgaagct atgtccaagc | |
| 14941 | gcaaaatcaa agaagagatg ctccaggtca tcgcgccgga gatctatggc cccccgaaga | |
| 15001 | aggaagagca ggattacaag ccccgaaagc taaagcgggt caaaaagaaa aagaaagatg | |
| 15061 | atgatgatga acttgacgac gaggtggaac tgctgcacgc taccgcgccc aggcgacggg | |
| 15121 | tacagtggaa aggtcgacgc gtaaaacgtg ttttgcgacc cggcaccacc gtagtcttta | |
| 15181 | cgcccggtga gcgctccacc cgcacctaca agcgcgtgta tgatgaggtg tacggcgacg | |
| 15241 | aggacctgct tgagcaggcc aacgagcgcc tcggggagtt tgcctacgga aagcggcata | |
| 15301 | aggacatgct ggcgttgccg ctggacgagg gcaacccaac acctagccta aagcccgtaa | |
| 15361 | cactgcagca ggtgctgccc gcgcttgcac cgtccgaaga aaagcgcggc ctaaagcgcg | |
| 15421 | agtctggtga cttggcaccc accgtgcagc tgatggtacc caagcgccag cgactggaag | |
| 15481 | atgtcttgga aaaaatgacc gtggaacctg ggctggagcc cgaggtccgc gtgcggccaa | |
| 15541 | tcaagcaggt ggcgccggga ctgggcgtgc agaccgtgga cgttcagata cccactacca | |
| 15601 | gtagcaccag tattgccacc gccacagagg gcatggagac acaaacgtcc ccggttgcct | |
| 15661 | cagcggtggc ggatgccgcg gtgcaggcgg tcgctgcggc cgcgtccaag acctctacgg | |
| 15721 | aggtgcaaac ggacccgtgg atgtttcgcg tttcagcccc ccggcgcccg cgcggttcga | |
| 15781 | ggaagtacgg cgccgccagc gcgctactgc ccgaatatgc cctacatcct tccattgcgc | |
| 15841 | ctacccccgg ctatcgtggc tacacctacc gccccagaag acgagcaact acccgacgcc | |
| 15901 | gaaccaccac tggaacccgc cgccgccgtc gccgtcgcca gcccgtgctg gccccgattt | |
| 15961 | ccgtgcgcag ggtggctcgc gaaggaggca ggaccctggt gctgccaaca gcgcgctacc | |
| 16021 | accccagcat cgtttaaaag ccggtctttg tggttcttgc agatatggcc ctcacctgcc | |
| 16081 | gcctccgttt cccggtgccg ggattccgag gaagaatgca ccgtaggagg ggcatggccg | |
| 16141 | gccacggcct gacgggcggc atgcgtcgtg cgcaccaccg gcggcggcgc gcgtcgcacc | |
| 16201 | gtcgcatgcg cggcggtatc ctgcccctcc ttattccact gatcgccgcg gcgattggcg | |
| 16261 | ccgtgcccgg aattgcatcc gtggccttgc aggcgcagag acactgatta aaaacaagtt | |
| 16321 | gcatgtggaa aaatcaaaat aaaaagtctg gactctcacg ctcgcttggt cctgtaacta | |
| 16381 | ttttgtagaa tggaagacat caactttgcg tctctggccc cgcgacacgg ctcgcgcccg | |
| 16441 | ttcatgggaa actggcaaga tatcggcacc agcaatatga gcggtggcgc cttcagctgg | |
| 16501 | ggctcgctgt ggagcggcat taaaaatttc ggttccaccg ttaagaacta tggcagcaag | |
| 16561 | gcctggaaca gcagcacagg ccagatgctg agggataagt tgaaagagca aaatttccaa | |
| 16621 | caaaaggtgg tagatggcct ggcctctggc attagcgggg tggtggacct ggccaaccag | |
| 16681 | gcagtgcaaa ataagattaa cagtaagctt gatccccgcc ctcccgtaga ggagcctcca | |
| 16741 | ccggccgtgg agacagtgtc tccagagggg cgtggcgaaa agcgtccgcg ccccgacagg | |
| 16801 | gaagaaatct ggtgacgcaa atagacgagc ctccctcgta cgaggaggca ctaaagcaag | |
| 16861 | gcctgcccac cacccgtccc atcgcgccca tggctaccgg agtgctgggc cagcacacac | |
| 16921 | ccgtaacgct ggacctgcct ccccccgccg acacccagca gaaacctgtg ctgccaggcc | |
| 16981 | cgaccgccgt tgttgtaacc cgtcctagcc gcgcgtccct gcgccgcgcc gccagcggtc | |
| 17041 | cgcgatcgtt gcggcccgta gccagtggca actggcaaag cacactgaac agcatcgtgg | |
| 17101 | gtctgggggt gcaatccctg aagcgccgac gatgcttctg aatagctaac gtgtcgtatg | |
| 17161 | tgtgtcatgt atgcgtccat gtcgccgcca gaggagctgc tgagccgccg cgcgcccgct | |
| 17221 | ttccaagatg gctacccctt cgatgatgcc gcagtggtct tacatgcaca tctcgggcca | |
| 17281 | ggacgcctcg gagtacctga gccccgggct ggtgcagttt gcccgcgcca ccgagacgta | |
| 17341 | cttcagcctg aataacaagt ttagaaaccc cacggtggcg cctacgcacg acgtgaccac | |
| 17401 | agaccggtcc cagcgtttga cgctgcggtt catccctgtg gaccgtgagg atactgcgta | |
| 17461 | ctcgtacaag gcgcggttca ccctagctgt gggtgataac cgtgtgctgg acatggcttc | |
| 17521 | cacgtacttt gacatccgcg gcgtgctgga caggggccct acttttaagc cctactctgg | |
| 17581 | cactgcctac aacgccctgg ctcccaaggg tgccccaaat ccttgcgaat gggatgaagc | |
| 17641 | tgctactgct cttgaaataa acctagaaga agaggacgat gacaacgaag acgaagtaga | |
| 17701 | cgagcaagct gagcagcaaa aaactcacgt atttgggcag gcgccttatt ctggtataaa | |
| 17761 | tattacaaag gagggtattc aaataggtgt cgaaggtcaa acacctaaat atgccgataa | |
| 17821 | aacatttcaa cctgaacctc aaataggaga atctcagtgg tacgaaactg aaattaatca | |
| 17881 | tgcagctggg agagtcctta aaaagactac cccaatgaaa ccatgttacg gttcatatgc | |
| 17941 | aaaacccaca aatgaaaatg gagggcaagg cattcttgta aagcaacaaa atggaaagct | |
| 18001 | agaaagtcaa gtggaaatgc aatttttctc aactactgag gcgaccgcag gcaatggtga | |
| 18061 | taacttgact cctaaagtgg tattgtacag tgaagatgta gatatagaaa ccccagacac | |
| 18121 | tcatatttct tacatgccca ctattaagga aggtaactca cgagaactaa tgggccaaca | |
| 18181 | atctatgccc aacaggccta attacattgc ttttagggac aattttattg gtctaatgta | |
| 18241 | ttacaacagc acgggtaata tgggtgttct ggcgggccaa gcatcgcagt tgaatgctgt | |
| 18301 | tgtagatttg caagacagaa acacagagct ttcataccag cttttgcttg attccattgg | |
| 18361 | tgatagaacc aggtactttt ctatgtggaa tcaggctgtt gacagctatg atccagatgt | |
| 18421 | tagaattatt gaaaatcatg gaactgaaga tgaacttcca aattactgct ttccactggg | |
| 18481 | aggtgtgatt aatacagaga ctcttaccaa ggtaaaacct aaaacaggtc aggaaaatgg | |
| 18541 | atgggaaaaa gatgctacag aattttcaga taaaaatgaa ataagagttg gaaataattt | |
| 18601 | tgccatggaa atcaatctaa atgccaacct gtggagaaat ttcctgtact ccaacatagc | |
| 18661 | gctgtatttg cccgacaagc taaagtacag tccttccaac gtaaaaattt ctgataaccc | |
| 18721 | aaacacctac gactacatga acaagcgagt ggtggctccc gggttagtgg actgctacat | |
| 18781 | taaccttgga gcacgctggt cccttgacta tatggacaac gtcaacccat ttaaccacca | |
| 18841 | ccgcaatgct ggcctcgcta ccgctcaatg ttgctgggca atggtcgcta tgtgcccttc | |
| 18901 | cacatccagg tgcctcagaa gttctttgcc attaaaaacc tccttctcct gccgggctca | |
| 18961 | tacacctacg agtggaactt caggaaggat gttaacatgg ttctgcagag ctccctagga | |
| 19021 | aatgacctaa gggttgacgg agccagcatt aagtttgata gcatttgcct ttacgccacc | |
| 19081 | ttcttcccca tggcccacaa caccgcctcc acgcttgagg ccatgcttag aaacgacacc | |
| 19141 | aacgaccagt cctttaacga ctatctctcc gccgccaaca tgctctaccc tatacccgcc | |
| 19201 | aacgctacca acgtgcccat atccatcccc tcccgcaact gggcggcttt ccgcggctgg | |
| 19261 | gccttcacgc gccttaagac taaggaaacc ccatcactgg gctcgggcta cgacccttat | |
| 19321 | tacacctact ctggctctat accctaccta gatggaacct tttacctcaa ccacaccttt | |
| 19381 | aagaaggtgg ccattacctt tgactcttct gtcagctggc ctggcaatga ccgcctgctt | |
| 19441 | acccccaacg agtttgaaat taagcgctca gttgacgggg agggttacaa cgttgcccag | |
| 19501 | tgtaacatga ccaaagactg gttcctggta caaatgctag ctaactacaa cattggctac | |
| 19561 | cagggcttct atatcccaga gagctacaag gaccgcatgt actccttctt tagaaacttc | |
| 19621 | cagcccatga gccgtcaggt ggtggatgat actaaataca aggactacca acaggtgggc | |
| 19681 | atcctacacc aacacaacaa ctctggattt gttggctacc ttgcccccac catgcgcgaa | |
| 19741 | ggacaggcct accctgctaa cttcccctat ccgcttatag gcaagaccgc agttgacagc | |
| 19801 | attacccaga aaaagtttct ttgcgatcgc accctttggc gcatcccatt ctccagtaac | |
| 19861 | tttatgtcca tgggcgcact cacagacctg ggccaaaacc ttctctacgc caactccgcc | |
| 19921 | cacgcgctag acatgacttt tgaggtggat cccatggacg agcccaccct tctttatgtt | |
| 19981 | ttgtttgaag tctttgacgt ggtccgtgtg caccggccgc accgcggcgt catcgaaacc | |
| 20041 | gtgtacctgc gcacgccctt ctcggccggc aacgccacaa cataaagaag caagcaacat | |
| 20101 | caacaacagc tgccgccatg ggctccagtg agcaggaact gaaagccatt gtcaaagatc | |
| 20161 | ttggttgtgg gccatatttt ttgggcacct atgacaagcg ctttccaggc tttgtttctc | |
| 20221 | cacacaagct cgcctgcgcc atagtcaata cggccggtcg cgagactggg ggcgtacact | |
| 20281 | ggatggcctt tgcctggaac ccgcactcaa aaacatgcta cctctttgag ccctttggct | |
| 20341 | tttctgacca gcgactcaag caggtttacc agtttgagta cgagtcactc ctgcgccgta | |
| 20401 | gcgccattgc ttcttccccc gaccgctgta taacgctgga aaagtccacc caaagcgtac | |
| 20461 | aggggcccaa ctcggccgcc tgtggactat tctgctgcat gtttctccac gcctttgcca | |
| 20521 | actggcccca aactcccatg gatcacaacc ccaccatgaa ccttattacc ggggtaccca | |
| 20581 | actccatgct caacagtccc caggtacagc ccaccctgcg tcgcaaccag gaacagctct | |
| 20641 | acagcttcct ggagcgccac tcgccctact tccgcagcca cagtgcgcag attaggagcg | |
| 20701 | ccacttcttt ttgtcacttg aaaaacatgt aaaaataatg tactagagac actttcaata | |
| 20761 | aaggcaaatg cttttatttg tacactctcg ggtgattatt tacccccacc cttgccgtct | |
| 20821 | gcgccgttta aaaatcaaag gggttctgcc gcgcatcgct atgcgccact ggcagggaca | |
| 20881 | cgttgcgata ctggtgttta gtgtccactt aaactcaggc acaaccatcc gcggcagctc | |
| 20941 | ggtgaagttt tcactccaca ggctgcgcac catcaccaac gcgtttagca ggtcgggcgc | |
| 21001 | cgatatcttg aagtcgcagt tggggcctcc gccctgcgcg cgcgagttgc gatacacagg | |
| 21061 | gttgcagcac tggaacacta tcagcgccgg gtggtgcacg ctggccagca cgctcttgtc | |
| 21121 | ggagatcaga tccgcgtcca ggtcctccgc gttgctcagg gcgaacggag tcaactttgg | |
| 21181 | tagctgcctt cccaaaaagg gcgcgtgccc aggctttgag ttgcactcgc accgtagtgg | |
| 21241 | catcaaaagg tgaccgtgcc cggtctgggc gttaggatac agcgcctgca taaaagcctt | |
| 21301 | gatctgctta aaagccacct gagcctttgc gccttcagag aagaacatgc cgcaagactt | |
| 21361 | gccggaaaac tgattggccg gacaggccgc gtcgtgcacg cagcaccttg cgtcggtgtt | |
| 21421 | ggagatctgc accacatttc ggccccaccg gttcttcacg atcttggcct tgctagactg | |
| 21481 | ctccttcagc gcgcgctgcc cgttttcgct cgtcacatcc atttcaatca cgtgctcctt | |
| 21541 | atttatcata atgcttccgt gtagacactt aagctcgcct tcgatctcag cgcagcggtg | |
| 21601 | cagccacaac gcgcagcccg tgggctcgtg atgcttgtag gtcacctctg caaacgactg | |
| 21661 | caggtacgcc tgcaggaatc gccccatcat cgtcacaaag gtcttgttgc tggtgaaggt | |
| 21721 | cagctgcaac ccgcggtgct cctcgttcag ccaggtcttg catacggccg ccagagcttc | |
| 21781 | cacttggtca ggcagtagtt tgaagttcgc ctttagatcg ttatccacgt ggtacttgtc | |
| 21841 | catcagcgcg cgcgcagcct ccatgccctt ctcccacgca gacacgatcg gcacactcag | |
| 21901 | cgggttcatc accgtaattt cactttccgc ttcgctgggc tcttcctctt cctcttgcgt | |
| 21961 | ccgcatacca cgcgccactg ggtcgtcttc attcagccgc cgcactgtgc gcttacctcc | |
| 22021 | tttgccatgc ttgattagca ccggtgggtt gctgaaaccc accatttgta gcgccacatc | |
| 22081 | ttctctttct tcctcgctgt ccacgattac ctctggtgat ggcgggcgct cgggcttggg | |
| 22141 | agaagggcgc ttctttttct tcttgggcgc aatggccaaa tccgccgccg aggtcgatgg | |
| 22201 | ccgcgggctg ggtgtgcgcg gcaccagcgc gtcttgtgat gagtcttcct cgtcctcgga | |
| 22261 | ctcgatacgc cgcctcatcc gcttttttgg gggcgcccgg ggaggcggcg gcgacgggga | |
| 22321 | cggggacgac acgtcctcca tggttggggg acgtcgcgcc gcaccgcgtc cgcgctcggg | |
| 22381 | ggtggtttcg cgctgctcct cttcccgact ggccatttcc ttctcctata ggcagaaaaa | |
| 22441 | gatcatggag tcagtcgaga agaaggacag cctaaccgcc ccctctgagt tcgccaccac | |
| 22501 | cgcctccacc gatgccgcca acgcgcctac caccttcccc gtcgaggcac ccccgcttga | |
| 22561 | ggaggaggaa gtgattatcg agcaggaccc aggttttgta agcgaagacg acgaggaccg | |
| 22621 | ctcagtacca acagaggata aaaagcaaga ccaggacaac gcagaggcaa acgaggaaca | |
| 22681 | agtcgggcgg ggggacgaaa ggcatggcga ctacctagat gtgggagacg acgtgctgtt | |
| 22741 | gaagcatctg cagcgccagt gcgccattat ctgcgacgcg ttgcaagagc gcagcgatgt | |
| 22801 | gcccctcgcc atagcggatg tcagccttgc ctacgaacgc cacctattct caccgcgcgt | |
| 22861 | accccccaaa cgccaagaaa acggcacatg cgagcccaac ccgcgcctca acttctaccc | |
| 22921 | cgtatttgcc gtgccagagg tgcttgccac catcacatct ttttccaaaa ctgcaagata | |
| 22981 | cccctatcct gccgtgccaa ccgcagccga gcggacaagc agctggcctt gcggcagggc | |
| 23041 | gctgtcatac ctgatatcgc ctcgctcaac gaagtgccaa aaatctttga gggtcttgga | |
| 23101 | cgcgacgaga agcgcgcggc aaacgctctg caacaggaaa acagcgaaaa tgaaagtcac | |
| 23161 | tctggagtgt tggtggaact cgagggtgac aacgcgcgcc tagccgtact aaaacgcagc | |
| 23221 | atcgaggtca cccactttgc ctacccggca cttaacctac cccccaaggt catgagcaca | |
| 23281 | gtcatgagtg agctgatcgt gcgccgtgcg cagcccctgg agagggatgc aaatttgcaa | |
| 23341 | gaacaaacag aggagggcct acccgcagtt ggcgacgagc agctagcgcg ctggcttcaa | |
| 23401 | acgcgcgagc ctgccgactt ggaggagcga cgcaaactaa tgatggccgc agtgctcgtt | |
| 23461 | accgtggagc ttgagtgcat gcagcggttc tttgctgacc cggagatgca gcgcaagcta | |
| 23521 | gaggaaacat tgcactacac ctttcgacag ggctacgtac gccaggcctg caagatctcc | |
| 23581 | aacgtggagc tctgcaacct ggtctcctac cttggaattt tgcacgaaaa ccgccttggg | |
| 23641 | caaaacgtgc ttcattccac gctcaagggc gaggcgcgcc gcgactacgt ccgcgactgc | |
| 23701 | gtttacttat ttctatgcta cacctggcag acggccatgg gcgtttggca gcagtgcttg | |
| 23761 | gaggagtgca acctcaagga gctgcagaaa ctgctaaagc aaaacttgaa ggacctatgg | |
| 23821 | acggccttca acgagcgctc cgtggccgcg cacctggcgg acatcatttt ccccgaacgc | |
| 23881 | ctgcttaaaa ccctgcaaca gggtctgcca gacttcacca gtcaaagcat gttgcagaac | |
| 23941 | tttaggaact ttatcctaga gcgctcagga atcttgcccg ccacctgctg tgcacttcct | |
| 24001 | agcgactttg tgcccattaa gtaccgcgaa tgccctccgc cgctttgggg ccactgctac | |
| 24061 | cttctgcagc tagccaacta ccttgcctac cactctgaca taatggaaga cgtgagcggt | |
| 24121 | gacggtctac tggagtgtca ctgtcgctgc aacctatgca ccccgcaccg ctccctggtt | |
| 24181 | tgcaattcgc agctgcttaa cgaaagtcaa attatcggta cctttgagct gcagggtccc | |
| 24241 | tcgcctgacg aaaagtccgc ggctccgggg ttgaaactca ctccggggct gtggacgtcg | |
| 24301 | gcttaccttc gcaaatttgt acctgaggac taccacgccc acgagattag gttctacgaa | |
| 24361 | gaccaatccc gcccgccaaa tgcggagctt accgcctgcg tcattaccca gggccacatt | |
| 24421 | cttggccaat tgcaagccat caacaaagcc cgccaagagt ttctgctacg aaagggacgg | |
| 24481 | ggggtttact tggaccccca gtccggcgag gagctcaacc caatcccccc gccgccgcag | |
| 24541 | ccctatcagc agcagccgcg ggcccttgct tcccaggatg gcacccaaaa agaagctgca | |
| 24601 | gctgccgccg ccacccacgg acgaggagga atactgggac agtcaggcag aggaggtttt | |
| 24661 | ggacgaggag gaggaggaca tgatggaaga ctgggagagc ctagacgagg aagcttccga | |
| 24721 | ggtcgaagag gtgtcagacg aaacaccgtc accctcggtc gcattcccct cgccggcgcc | |
| 24781 | ccagaaatcg gcaaccggtt ccagcatggc tacaacctcc gctcctcagg cgccgccggc | |
| 24841 | actgcccgtt cgccgaccca accgtagatg ggacaccact ggaaccaggg ccggtaagtc | |
| 24901 | caagcagccg ccgccgttag cccaagagca acaacagcgc caaggctacc gctcatggcg | |
| 24961 | cgggcacaag aacgccatag ttgcttgctt gcaagactgg ggggcaacat ctccttcgcc | |
| 25021 | cgccgctttc ttctctacca tcacggcgtg gccttccccc gtaacatcct gcattactac | |
| 25081 | cgtcatctct acagcccata ctgcaccggc ggcagcggca gcggcagcaa cagcagcggc | |
| 25141 | cacacagaag caaaggcgac cggatagcaa gactctgaca aagcccaaga aatccacagc | |
| 25201 | ggcggcagca gcaggaggag gagcgctgcg tctggcgccc aacgaacccg tatcgacccg | |
| 25261 | cgagcttaga aacaggattt ttcccactct gtatgctata tttcaacaga gcaggggcca | |
| 25321 | agaacaagag ctgaaaataa aaaacaggtc tctgcgatcc ctcacccgca gctgcctgta | |
| 25381 | tcacaaaagc gaagatcagc ttcggcgcac gctggaagac gcggaggctc tcttcagtaa | |
| 25441 | atactgcgcg ctgactctta aggactagtt tcgcgccctt tctcaaattt aagcgcgaaa | |
| 25501 | actacgtcat ctccagcggc cacacccggc gccagcacct gtcgtcagcg ccattatgag | |
| 25561 | caaggaaatt cccacgccct acatgtggag ttaccagcca caaatgggac ttgcggctgg | |
| 25621 | agctgcccaa gactactcaa cccgaataaa ctacatgagc gcgggacccc acatgatatc | |
| 25681 | ccgggtcaac ggaatccgcg cccaccgaaa ccgaattctc ttggaacagg cggctattac | |
| 25741 | caccacacct cgtaataacc ttaatccccg tagttggccc gctgccctgg tgtaccagga | |
| 25801 | aagtcccgct cccaccactg tggtacttcc cagagacgcc caggccgaag ttcagatgac | |
| 25861 | taactcaggg gcgcagcttg cgggcggctt tcgtcacagg gtgcggtcgc ccgggcaggg | |
| 25921 | tataactcac ctgacaatca gagggcgagg tattcagctc aacgacgagt cggtgagctc | |
| 25981 | ctcgcttggt ctccgtccgg acgggacatt tcagatcggc ggcgccggcc gctcttcatt | |
| 26041 | cacgcctcgt caggcaatcc taactctgca gacctcgtcc tctgagccgc gctctggagg | |
| 26101 | cattggaact ctgcaattta ttgaggagtt tgtgccatcg gtctacttta accccttctc | |
| 26161 | gggacctccc ggccactatc cggatcaatt tattcctaac tttgacgcgg taaaggactc | |
| 26221 | ggcggatggc tacgactgaa tgttaagtgg agaggcagag caactgcgcc tgaaacacct | |
| 26281 | ggtccactgt cgccgccaca agtgctttgc ccgcgactcc ggtgagtttt gctactttga | |
| 26341 | attgcccgag gatcatatcg agggcccggc gcacggcgtc cggcttaccg cccagggaga | |
| 26401 | gcttgcccgt agcctgattc gggagtttac ccagcgcccc ctgctagttg agcgggacag | |
| 26461 | gggaccctgt gttctcactg tgatttgcaa ctgtcctaac cctggattac atcaagatct | |
| 26521 | ttgttgccat ctctgtgctg agtataataa atacagaaat taaaatatac tggggctcct | |
| 26581 | atcgccatcc tgtaaacgcc accgtcttca cccgcccaag caaaccaagg cgaaccttac | |
| 26641 | ctggtacttt taacatctct ccctctgtga tttacaacag tttcaaccca gacggagtga | |
| 26701 | gtctacgaga gaacctctcc gagctcagct actccatcag aaaaaacacc accctcctta | |
| 26761 | cctgccggga acgtacgagt gcgtcaccgg ccgctgcacc acacctaccg cctgaccgta | |
| 26821 | aaccagactt tttccggaca gacctcaata actctgttta ccagaacagg aggtgagctt | |
| 26881 | agaaaaccct tagggtatta ggccaaaggc gcagctactg tggggtttat gaacaattca | |
| 26941 | agcaactcta cgggctattc taattcaggt ttctctagaa tcggggttgg ggttattctc | |
| 27001 | tgtcttgtga ttctctttat tcttatacta acgcttctct gcctaagctc gccgcctgct | |
| 27061 | gtgtgcacat ttgcatttat tgtcagcttt ttaaacgctg gggtcgccac ccaagatgat | |
| 27121 | taggtacata atcctaggtt tactcaccct tgcgtcagcc cacggtacca cccaaaaggt | |
| 27181 | ggattttaag gagccagcct gtaatgttac attcgcagct gaagctaatg agtgcaccac | |
| 27241 | tcttataaaa tgcaccacag aacatgaaaa gctgcttatt cgccacaaaa acaaaattgg | |
| 27301 | caagtatgct gtttatgcta tttggcagcc aggtgacact acagagtata atgttacagt | |
| 27361 | tttccagggt aaaagtcata aaacttttat gtatactttt ccattttatg aaatgtgcga | |
| 27421 | cattaccatg tacatgagca aacagtataa gttgtggccc ccacaaaatt gtgtggaaaa | |
| 27481 | cactggcact ttctgctgca ctgctatgct aattacagtg ctcgctttgg tctgtaccct | |
| 27541 | actctatatt aaatacaaaa gcagacgcag ctttattgag gaaaagaaaa tgccttaatt | |
| 27601 | tactaagtta caaagctaat gtcaccacta actgctttac tcgctgcttg caaaacaaat | |
| 27661 | tcaaaaagtt agcattataa ttagaatagg atttaaaccc cccggtcatt tcctgctcaa | |
| 27721 | taccattccc ctgaacaatt gactctatgt gggatatgct ccagcgctac aaccttgaag | |
| 27781 | tcaggcttcc tggatgtcag catctgactt tggccagcac ctgtcccgcg gatttgttcc | |
| 27841 | agtccaacta cagcgaccca ccctaacaga gatgaccaac acaaccaacg cggccgccgc | |
| 27901 | taccggactt acatctacca caaatacacc ccaagtttct gcctttgtca ataactggga | |
| 27961 | taacttgggc atgtggtggt tctccatagc gcttatgttt gtatgcctta ttattatgtg | |
| 28021 | gctcatctgc tgcctaaagc gcaaacgcgc ccgaccaccc atctatagtc ccatcattgt | |
| 28081 | gctacaccca aacaatgatg gaatccatag attggacgga ctgaaacaca tgttcttttc | |
| 28141 | tcttacagta tgattaaatg agacatgatt cctcgagttt ttatattact gacccttgtt | |
| 28201 | gcgctttttt gtgcgtgctc cacattggct gcggtttctc acatcgaagt agactgcatt | |
| 28261 | ccagccttca cagtctattt gctttacgga tttgtcaccc tcacgctcat ctgcagcctc | |
| 28321 | atcactgtgg tcatcgcctt tatccagtgc attgactggg tctgtgtgcg ctttgcatat | |
| 28381 | ctcagacacc atccccagta cagggacagg actatagctg agcttcttag aattctttaa | |
| 28441 | ttatgaaatt tactgtgact tttctgctga ttatttgcac cctatctgcg ttttgttccc | |
| 28501 | cgacctccaa gcctcaaaga catatatcat gcagattcac tcgtatatgg aatattccaa | |
| 28561 | gttgctacaa tgaaaaaagc gatctttccg aagcctggtt atatgcaatc atctctgtta | |
| 28621 | tggtgttctg cagtaccatc ttagccctag ctatatatcc ctaccttgac attggctgga | |
| 28681 | aacgaataga tgccatgaac cacccaactt tccccgcgcc cgctatgctt ccactgcaac | |
| 28741 | aagttgttgc cggcggcttt gtcccagcca atcagcctcg ccccacttct cccaccccca | |
| 28801 | ctgaaatcag ctactttaat ctaacaggag gagatgactg acaccctaga tctagaaatg | |
| 28861 | gacggaatta ttacagagca gcgcctgcta gaaagacgca gggcagcggc cgagcaacag | |
| 28921 | cgcatgaatc aagagctcca agacatggtt aacttgcacc agtgcaaaag gggtatcttt | |
| 28981 | tgtctggtaa agcaggccaa agtcacctac gacagtaata ccaccggaca ccgccttagc | |
| 29041 | tacaagttgc caaccaagcg tcagaaattg gtggtcatgg tgggagaaaa gcccattacc | |
| 29101 | ataactcagc actcggtaga aaccgaaggc tgcattcact caccttgtca aggacctgag | |
| 29161 | gatctctgca cccttattaa gaccctgtgc ggtctcaaag atcttattcc ctttaactaa | |
| 29221 | taaaaaaaaa taataaagca tcacttactt aaaatcagtt agcaaatttc tgtccagttt | |
| 29281 | attcagcagc acctccttgc cctcctccca gctctggtat tgcagcttcc tcctggctgc | |
| 29341 | aaactttctc cacaatctaa atggaatgtc agtttcctcc tgttcctgtc catccgcacc | |
| 29401 | cactatcttc atgttgttgc agatgaagcg cgcaagaccg tctgaagata ccttcaaccc | |
| 29461 | cgtgtatcca tatgacacgg aaaccggtcc tccaactgtg ccttttctta ctcctccctt | |
| 29521 | tgtatccccc aatgggtttc aagagagtcc ccctggggta ctctctttgc gcctatccga | |
| 29581 | acctctagtt acctccaatg gcatgcttgc gctcaaaatg ggcaacggcc tctctctgga | |
| 29641 | cgaggccggc aaccttacct cccaaaatgt aaccactgtg agcccacctc tcaaaaaaac | |
| 29701 | caagtcaaac ataaacctgg aaatatctgc acccctcaca gttacctcag aagccctaac | |
| 29761 | tgtggctgcc gccgcacctc taatggtcgc gggcaacaca ctcaccatgc aatcacaggc | |
| 29821 | cccgctaacc gtgcacgact ccaaacttag cattgccacc caaggacccc tcacagtgtc | |
| 29881 | agaaggaaag ctagccctgc aaacatcagg ccccctcacc accaccgata gcagtaccct | |
| 29941 | tactatcact gcctcacccc ctctaactac tgccactggt agcttgggca ttgacttgaa | |
| 30001 | agagcccatt tatacacaaa atggaaaact aggactaaag tacggggctc ctttgcatgt | |
| 30061 | aacagacgac ctaaacactt tgaccgtagc aactggtcca ggtgtgacta ttaataatac | |
| 30121 | ttccttgcaa actaaagtta ctggagcctt gggttttgat tcacaaggca atatgcaact | |
| 30181 | taatgtagca ggaggactaa ggattgattc tcaaaacaga cgccttatac ttgatgttag | |
| 30241 | ttatccgttt gatgctcaaa accaactaaa tctaagacta ggacagggcc ctctttttat | |
| 30301 | aaactcagcc cacaacttgg atattaacta caacaaaggc ctttacttgt ttacagcttc | |
| 30361 | aaacaattcc aaaaagcttg aggttaacct aagcactgcc aaggggttga tgtttgacgc | |
| 30421 | tacagccata gccattaatg caggagatgg gcttgaattt ggttcaccta atgcaccaaa | |
| 30481 | cacaaatccc ctcaaaacaa aaattggcca tggcctagaa tttgattcaa acaaggctat | |
| 30541 | ggttcctaaa ctaggaactg gccttagttt tgacagcaca ggtgccatta cagtaggaaa | |
| 30601 | caaaaataat gataagctaa ctttgtggac cacaccagct ccatctccta actgtagact | |
| 30661 | aaatgcagag aaagatgcta aactcacttt ggtcttaaca aaatgtggca gtcaaatact | |
| 30721 | tgctacagtt tcagttttgg ctgttaaagg cagtttggct ccaatatctg gaacagttca | |
| 30781 | aagtgctcat cttattataa gatttgacga aaatggagtg ctactaaaca attccttcct | |
| 30841 | qgacccagaa tattggaact ttagaaatgg agatcttact gaaggcacag cctatacaaa | |
| 30901 | cgctgttgga tttatgccta acctatcagc ttatccaaaa tctcacggta aaactgccaa | |
| 30961 | aagtaacatt gtcagtcaag tttacttaaa cggagacaaa actaaacctg taacactaac | |
| 31021 | cattacacta aacggtacac aggaaacagg agacacaact ccaagtgcat actctatgtc | |
| 31081 | attttcatgg gactggtctg gccacaacta cattaatgaa atatttgcca catcctctta | |
| 31141 | cactttttca tacattgccc aagagggtgg aggcggttca ggcggaggtg gctctggcgg | |
| 31201 | tggcggatcc gcggataaca aattcaacaa agaacaacaa aatgctttct atgaaatctt | |
| 31261 | acatttacct aacttaaacg aagaacaacg taacgcattc atccaaagcc ttaaagacga | |
| 31321 | tccttcagtg agcaaagaaa ttttagcaga agctaaaaag ctaaacgatg ctcaagcacc | |
| 31381 | aaaataataa atgaatcgtt tgtgttatgt ttcaacgtgt ttatttttca attgcagaaa | |
| 31441 | atttcaagtc atttttcatt cagtagtata gccccaccac cacatagctt atacagatca | |
| 31501 | ccgtacctta atcaaactca cagaacccta gtattcaacc tgccacctcc ctcccaacac | |
| 31561 | acagagtaca cagtcctttc tccccggctg gccttaaaaa gcatcatatc atgggtaaca | |
| 31621 | gacatattct taggtgttat attccacacg gtttcctgtc gagccaaacg ctcatcagtg | |
| 31681 | atattaataa actccccggg cagctcactt aagttcatgt cgctgtccag ctgctgagcc | |
| 31741 | acaggctgct gtccaacttg cggttgctta acgggcggcg aaggagaagt ccacgcctac | |
| 31801 | atgggggtag agtcataatc gtgcatcagg atagggcggt ggtgctgcag cagcgcgcga | |
| 31861 | ataaactgct gccgccgccg ctccgtcctg caggaataca acatggcagt ggtctcctca | |
| 31921 | gcgatgattc gcaccgcccg cagcataagg cgccttgtcc tccgggcaca gcagcgcacc | |
| 31981 | ctgatctcac ttaaatcagc acagtaactg cagcacagca ccacaatatt gttcaaaatc | |
| 32041 | ccacagtgca aggcgctgta tccaaagctc atggcgggga ccacagaacc cacgtggcca | |
| 32101 | tcataccaca agcgcaggta gattaagtgg cgacccctca taaacacgct ggacataaac | |
| 32161 | attacctctt ttggcatgtt gtaattcacc acctcccggt accatataaa cctctgatta | |
| 32221 | aacatggcgc catccaccac catcctaaac cagctggcca aaacctgccc gccggctata | |
| 32281 | cactgcaggg aaccgggact ggaacaatga cagtggagag cccaggactc gtaaccatgg | |
| 32341 | atcatcatgc tcgtcatgat atcaatgttg gcacaacaca ggcacacgtg catacacttc | |
| 32401 | ctcaggatta caagctcctc ccgcgttaga accatatccc agggaacaac ccattcctga | |
| 32461 | atcagcgtaa atcccacact gcagggaaga cctcgcacgt aactcacgtt gtgcattgtc | |
| 32521 | aaagtgttac attcgggcag cagcggatga tcctccagta tggtagcgcg ggtttctgtc | |
| 32581 | tcaaaaggag gtagacgatc cctactgtac ggagtgcgcc gagacaaccg agatcgtgtt | |
| 32641 | ggtcgtagtg tcatgccaaa tggaacgccg gacgtagtca tatttcctga agcaaaacca | |
| 32701 | ggtgcgggcg tgacaaacag atctgcgtct ccggtctcgc cgcttagatc gctctgtgta | |
| 32761 | gtagttgtag tatatccact ctctcaaagc atccaggcgc cccctggctt cgggttctat | |
| 32821 | gtaaactcct tcatgcgccg ctgccctgat aacatccacc accgcagaat aagccacacc | |
| 32881 | cagccaacct acacattcgt tctgcgagtc acacacggga ggagcgggaa gagctggaag | |
| 32941 | aaccatgttt ttttttttat tccaaaagat tatccaaaac ctcaaaatga agatctatta | |
| 33001 | agtgaacgcg ctcccctccg gtggcgtggt caaactctac agccaaagaa cagataatgg | |
| 33061 | catttgtaag atgttgcaca atggcttcca aaaggcaaac ggccctcacg tccaagtgga | |
| 33121 | cgtaaaggct aaacccttca gggtgaatct cctctataaa cattccagca ccttcaacca | |
| 33181 | tgcccaaata attctcatct cgccaccttc tcaatatatc tctaagcaaa tcccgaatat | |
| 33241 | taagtccggc cattgtaaaa atctgctcca gagcgccctc caccttcagc ctcaagcagc | |
| 33301 | gaatcatgat tgcaaaaatt caggttcctc acagacctgt ataagattca aaagcggaac | |
| 33361 | attaacaaaa ataccgcgat cccgtaggtc ccttcgcagg gccagctgaa cataatgtgc | |
| 33421 | aggtctgcac ggaccagcgc ggccacttcc ccgccaggaa ccatgacaaa agaacccaca | |
| 33481 | ctgattatga cacgcatact cggagctatg ctaaccagcg tagccccgat gtaagcttgt | |
| 33541 | tgcatgggcg gcgatataaa atgcaaggtg ctgctcaaaa aatcaggcaa agcctcgcgc | |
| 33601 | aaaaaagaaa gcacatcgta gtcatgctca tgcagataaa ggcaggtaag ctccggaacc | |
| 33661 | accacagaaa aagacaccat ttttctctca aacatgtctg cgggtttctg cataaacaca | |
| 33721 | aaataaaata acaaaaaaac atttaaacat tagaagcctg tcttacaaca ggaaaaacaa | |
| 33781 | cccttataag cataagacgg actacggcca tgccggcgtg accgtaaaaa aactggtcac | |
| 33841 | cgtgattaaa aagcaccacc gacagctcct cggtcatgtc cggagtcata atgtaagact | |
| 33901 | cggtaaacac atcaggttga ttcacatcgg tcagtgctaa aaagcgaccg aaatagcccg | |
| 33961 | ggggaataca tacccgcagg cgtagagaca acattacagc ccccatagga ggtataacaa | |
| 34021 | aattaatagg agagaaaaac acataaacac ctgaaaaacc ctcctgccta ggcaaaatag | |
| 34081 | caccctcccg ctccagaaca acatacagcg cttccacagc ggcagccata acagtcagcc | |
| 34141 | ttaccagtaa aaaagaaaac ctattaaaaa aacaccactc gacacggcac cagctcaatc | |
| 34201 | agtcacagtg taaaaaaggg ccaagtgcag agcgagtata tataggacta aaaaatgacg | |
| 34261 | taacggttaa agtccacaaa aaacacccag aaaaccgcac gcgaacctac gcccagaaac | |
| 34321 | gaaagccaaa aaacccacaa cttcctcaaa tcgtcacttc cgttttccca cgttacgtca | |
| 34381 | cttcccattt taagaaaact acaattccca acacatacaa gttactccgc cctaaaacct | |
| 34441 | acgtcacccg ccccgttccc acgccccgcg ccacgtcaca aactccaccc cctcattatc | |
| 34501 | atattggctt caatccaaaa taaggtatat tattgatgat g | |

Having thus described in detail advantageous embodiments of the present invention, it is to be understood that the invention defined by the above paragraphs is not to be limited to particular details set forth in the above description as many apparent variations thereof are possible without departing from the spirit or scope of the present invention.

1. A method for increasing binding affinity of a targeted ligand and a cell surface molecule that binds the targeted ligand comprising contacting the target ligand with a targeted recombinant adenovirus vector comprising: (i) a gene encoding a heterologous protein, (ii) a modified fiber protein comprising an immunoglobulin-binding domain and (iii) a gene encoding a fusion protein comprising a targeted ligand and an immunoglobulin Zc domain, wherein binding of the immunoglobulin-binding domain to the Zc domain connects the targeting ligand to the modified fiber protein, thereby targeting the adenovirus vector to a cell that expresses a cell surface molecule that binds to the targeted ligand and increasing the binding affinity of the targeted ligand and the cell surface molecule that binds the targeted ligand as compared to the binding of a binding of the targeted ligand with an adenovirus vector without an immunoglobulin Zc domain.

2. The method of claim 1 wherein the immunoglobulin-binding domain of the targeted adenovirus vector is inserted at the HI loop or the carboxy terminal of the fiber protein.

3. The method of claim 1 wherein the immunoglobulin-binding domain inserted at the HI loop is flanked by flexible linkers.

4. The method of claim 1 wherein the modified fiber protein comprises a fiber-fibritin chimera and the immunoglobulin-binding domain is inserted at the carboxy terminal of the fiber-fibritin chimera.

5. The method of claim 1 wherein the targeting ligand is a CD40 ligand or a single chain fragment (scFv) of anti-human CD40 antibody.

6. A method for increasing binding affinity of a targeted ligand and a cell surface molecule that binds the targeted ligand comprising contacting the target ligand with a CD40-targeted recombinant adenovirus vector comprising: (i) a gene encoding a heterologous protein, (ii) a modified fiber protein comprising an immunoglobulin-binding domain and (iii) a gene encoding a fusion protein comprising an immunoglobulin Zc domain and a targeting ligand selecting from the group consisting of CD40 ligand and a single chain fragment (scFv) of anti-human CD40 antibody, wherein binding of said immunoglobulin-binding domain to the Zc domain connects the targeting ligand to the modified fiber protein, thereby targeting the adenovirus vector to a CD40+ cell and increasing the binding affinity of the targeted ligand and the cell surface molecule that binds the targeted ligand as compared to the binding of a binding of the targeted ligand with an adenovirus vector without an immunoglobulin Zc domain.

7. The method of claim 6 wherein the immunoglobulin-binding domain is inserted at the HI loop or the carboxy terminal of the fiber protein.

8. The method of claim 6 wherein the immunoglobulin-binding domain inserted at the HI loop is flanked by flexible linkers.

9. The method of claim 6 wherein the modified fiber protein comprises a fiber-fibritin chimera and the immunoglobulin-binding domain is inserted at the carboxy terminal of the fiber-fibritin chimera.

10. The method of claim 6 wherein the CD40+ cell is a dendritic cell.

11. The method of claim 6 wherein the gene encoding the heterologous protein and the gene encoding the fusion protein are operably linked to a dendritic-cell-specific promoter.

12. A method for increasing increasing transduction effiency comprising contacting a targeted recombinant adenovirus vector comprising: (i) a gene encoding a heterologous protein, (ii) a modified fiber protein comprising an immunoglobulin-binding domain and (iii) a gene encoding a fusion protein comprising a targeted ligand and an immunoglobulin Zc domain, wherein binding of the immunoglobulin-binding domain to the Zc domain connects the targeting ligand to the modified fiber protein, to a cell that expresses a cell surface molecule that binds to the targeted ligand and increasing the transduction efficiency of the targeted recombinant adenovirus vector as compared an adenovirus vector without an immunoglobulin Zc domain.

13. The method of claim 12 wherein the immunoglobulin-binding domain of the targeted adenovirus vector is inserted at the HI loop or the carboxy terminal of the fiber protein.

14. The method of claim 12 wherein the immunoglobulin-binding domain inserted at the HI loop is flanked by flexible linkers.

15. The method of claim 12 wherein the modified fiber protein comprises a fiber-fibritin chimera and the immunoglobulin-binding domain is inserted at the carboxy terminal of the fiber-fibritin chimera.

16. The method of claim 12 wherein the targeting ligand is a CD40 ligand or a single chain fragment (scFv) of anti-human CD40 antibody.

17. A method for increasing increasing transduction effiency comprising contacting a a CD40-targeted recombinant adenovirus vector comprising: (i) a gene encoding a heterologous protein, (ii) a modified fiber protein comprising an immunoglobulin-binding domain and (iii) a gene encoding a fusion protein comprising an immunoglobulin Zc domain and a targeting ligand selecting from the group consisting of CD40 ligand and a single chain fragment (scFv) of anti-human CD40 antibody to a cell that expresses a cell surface molecule that binds to the targeted ligand and increasing the transduction efficiency of the targeted recombinant adenovirus vector as compared an adenovirus vector without an immunoglobulin Zc domain.

18. The method of claim 17 wherein the immunoglobulin-binding domain is inserted at the HI loop or the carboxy terminal of the fiber protein.

19. The method of claim 17 wherein the immunoglobulin-binding domain inserted at the HI loop is flanked by flexible linkers.

20. The method of claim 17 wherein the modified fiber protein comprises a fiber-fibritin chimera and the immunoglobulin-binding domain is inserted at the carboxy terminal of the fiber-fibritin chimera.

21. The method of claim 17 wherein the CD40+ cell is a dendritic cell.

22. The method of claim 17 wherein the gene encoding the heterologous protein and the gene encoding the fusion protein are operably linked to a dendritic-cell-specific promoter.

23. An adenovirus vector consisting essentially of the sequence of SEQ ID NO. 15.


SEQUENCE LISTING (16 sequences)

SEQ ID NO: 1 - protein, 15 aa, Artificial Sequence
  Description of Artificial Sequence Synthetic linker peptide
GGGGSGGGGSGGGGS

SEQ ID NO: 2 - DNA, 36 bp, Artificial Sequence
  Description of Artificial Sequence Synthetic oligonucleotide
acaactcggtggcggtaccggtgtatacggcggtcc

SEQ ID NO: 3 - DNA, 36 bp, Artificial Sequence
  Description of Artificial Sequence Synthetic oligonucleotide
ggaccgccgtatacaccggtaccgccaccgagttgt

SEQ ID NO: 4 - DNA, 40 bp, Artificial Sequence
  Description of Artificial Sequence Synthetic oligonucleotide
gatcccggtggcggtaccggtgtatacggcggttaataaa

SEQ ID NO: 5 - DNA, 40 bp, Artificial Sequence
  Description of Artificial Sequence Synthetic oligonucleotide
gatctttattaaccgccgtatacaccggtaccgccaccgg

SEQ ID NO: 6 - DNA, 12 bp, Artificial Sequence
  Description of Artificial Sequence Synthetic oligonucleotide
ctagccaattgg

SEQ ID NO: 7 - DNA, 90 bp, Artificial Sequence
  Description of Artificial Sequence Synthetic oligonucleotide
gcggataacaaattcaacaaagaacaacaaaatgctttctatgaaatcttacatttacctaacttaaacgaagaacaacgtaacggcttc

SEQ ID NO: 8 - DNA, 88 bp, Artificial Sequence
  Description of Artificial Sequence Synthetic oligonucleotide
gttacgttgttcttcgtttaagttaggtaaatgtaagatttcatagaaagcattttgttgttcttgttgaatttgttatccgcggatc

SEQ ID NO: 9 - DNA, 89 bp, Artificial Sequence
  Description of Artificial Sequence Synthetic oligonucleotide
atccaaagccttaaagacgatccttcagtgagcaaagaaattttagcagaagctaaaaagctaaacgatgctcaagcaccaaaataata

SEQ ID NO: 10 - DNA, 90 bp, Artificial Sequence
  Description of Artificial Sequence Synthetic oligonucleotide
ttttggtgcttgagcatcgtttagctttttagcttctgctaaaatttctttgctcactgaaggatcgtctttaaggctttggatgaagcc

SEQ ID NO: 11 - DNA, 20 bp, Artificial Sequence
  Description of Artificial Sequence Synthetic primer
acgtaacgcattcatccaaa

SEQ ID NO: 12 - DNA, 22 bp, Artificial Sequence
  Description of Artificial Sequence Synthetic primer
gacttgaaattttctgcaattg

SEQ ID NO: 13 - DNA, 21 bp, Artificial Sequence
  Description of Artificial Sequence Synthetic primer
ggtggcggatccgcggataac

SEQ ID NO: 14 - DNA, 20 bp, Artificial Sequence
  Description of Artificial Sequence Synthetic primer
tttggatgaatgcgttacgt

SEQ ID NO: 15 - DNA, 34541 bp, Human adenovirus type 5
  modified_base (4)..(6): a, c, g, t, unknown, or other
taannntcccttccagctctctgccccttttggattgaagccaatatgataatgagggggtggagtttgtgacgtggcgcgggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaannncgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccaacgcggaactccatatatgggctatgaactaatgaccccgtaattgattactattannntaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagaggggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggcctaacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgtactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggcattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaatctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgtccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccaccatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactggggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggatggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaagctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagagggtggaggcggttcaggcggaggtggctctggcggtggcggatccgcggataacaaattcaacaaagaacaacaaaatgctttctatgaaatcttacatttacctaacttaaacgaagaacaacgtaacgcattcatccaaagccttaaagacgatccttcagtgagcaaagaaattttagcagaagctaaaaagctaaacgatgctcaagcaccaaaataataaatgaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 16 - protein, 6 aa, Artificial Sequence
  Description of Artificial Sequence Synthetic 6xHis tag
HHHHHH